(19) United States
(12) Patent Application Publication
(10) Pub. No.: US 2026/0364230 A1
(43) Pub. Date: Nov. 19, 2026

(54) BONE SIALOPROTEIN COMPOSITIONS AND METHODS OF USING THEREOF

(71) Applicant: Ohio State Innovation Foundation, Columbus, OH (US)

(72) Inventors: Brian FOSTER, Hilliard, OH (US); Michael CHAVEZ, Coralville, IA (US)

(21) Appl. No.: 19/121,029

(22) PCT Filed: Oct. 10, 2023
(86) PCT No.: PCT/US2023/076484

§ 371 Date: Apr. 14, 2025

(51) Int. Cl. A61L 27/54; A61K 6/884; A61L 27/12; A61L 27/24; A61L 27/52; A61L 27/58

(52) U.S. Cl. CPC A61L 27/54; A61K 6/884; A61L 27/12; A61L 27/24; A61L 27/52; A61L 27/58; A61L 2300/252; A61L 2300/404; A61L 2300/414; A61L 2300/43; A61L 2430/02; A61L 2430/12

(57) ABSTRACT

Described herein are methods of using bone sialoprotein (BSP) to promote alveolar bone healing, for example, to improve bone preservation following tooth extraction, in preparation for a dental implant or other restorative device. Also described are methods of using BSP to improve osseointegration of dental implants to bone. Further described are methods of using BSP to promote periodontal repair and regeneration (e.g., to regenerate cementum and/or alveolar bone).

### CROSS-REFERENCE TO RELATED APPLICATIONS

This application claims benefit of U.S. Provisional Application No. 63/416,102, filed Oct. 14, 2023, which is hereby incorporated herein by reference in its entirety.

### STATEMENT REGARDING FEDERALLY SPONSORED RESEARCH OR DEVELOPMENT

This invention was made with Government Support under Grant No. R01 DE0276395 awarded by the National Institutes of Health. The Government has certain rights in the invention.

### BACKGROUND

Clinically, bone resorption in the maxillary and mandibular jaws occurs after loss of dentition. Partial edentulism affects 40% of the adult population and is estimated to increase in the next 15 years to more than 200 million individuals. In such cases, the bone resorption causes the alveolar ridge to decrease in width and height with a 50% loss in bone width occurring during the first year after a tooth is lost, two-thirds of which occur in the initial 3 months. The result of this is that before the patient's dentition is restored with dental implants, a separate procedure is required to replace this lost bone structure.

There are various surgical procedures available to graft the deficit alveolar ridge for both height and width. To do this a bone graft, commonly allograft bone powder/particulate or block is placed in the void space to provide osteoconductive/osteoinductive cues for targeted bone regeneration. Many of these procedures utilize a guided bone regenerative (GBR) membrane to maintain the bone graft in place as well as soft tissues. To date, optimal materials and methods for alveolar ridge bone grafting have yet to be developed. Thus, there remains a need for material that can support alveolar bone growth, promote alveolar bone and soft tissue healing, and inhibit infection.

### SUMMARY

Bone sialoprotein (BSP) is an extracellular matrix protein associated with mineralized tissues in teeth and in the skeleton. BSP is thought to be multifunctional, promoting mineralization of matrix and osteoblast differentiation.

As described herein, mice lacking BSP (Ibsp^{-/-} or Ibsp KO) have developmental defects in mineralization of cementum and alveolar bone, two mineralized tissues that are part of the periodontal complex that attaches the tooth to the jaw. Using a mouse model of molar tooth extraction, it was demonstrated that mice lacking BSP had defective alveolar bone healing. Furthermore, adding exogenous BSP during molar socket healing improved bone healing parameters in both mice deficient in BSP and normal control mice. Exogenous BSP included native rat BSP (nBSP, derived from rat bones) or recombinant rat BSP (rBSP; derived from bacterial expression of rat BSP). Both nBSP and rBSP improved alveolar bone healing and produced equivalent results.

Accordingly, provided herein are methods of using BSP to promote alveolar bone healing, for example, to improve bone preservation following tooth extraction, in preparation for a dental implant or other restorative device. Also described are methods of using BSP to improve osseointegration of dental implants. Further, both alveolar bone and cementum are reduced or damaged during periodontal disease. As BSP is important in the formation and function of both tissues, also provided are methods of using BSP to promote periodontal repair and regeneration (e.g., to regenerate cementum and/or alveolar bone). Furthermore, by determining importance of functional domains and post-translational modifications, the potential of BSP as a therapeutic may be optimized, e.g., by modifying BSP to include only the critical functional domains, such that the modified BSP outperform full length (native) BSP.

These and more methods are described in more detail below.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1. Reduced Extraction Time for Ibsp^{-/-} vs. WT Mouse Molars. Violin plot showing time (in 30 second intervals) to extract one first maxillary molar (M1). Ibsp^{-/-} mouse molars were perceived to be mobile by the operator and required significantly shorter time periods for extraction. ****P<0.0001

FIGS. 2A-2D. Creation of the Region of Interest for Analysis of Alveolar Bone Healing. (2A) A region of interest (ROI) calculated from the sockets of n=6 WT and Ibsp^{-/-} mice measured at 0 days post-procedure (dpp) was used to generate an average socket map. This average socket region was then eroded by 120 μm (yellow bars) to minimize inclusion of preexisting lamina dura and to localize the ROI entirely within the socket where only new bone formation would occur. The (2B) alveolar bone ROI was applied to (2C) scans of maxillae of WT and Ibsp^{-/-} mice at 0, 7, 21, and 56 dpp to define (2D) the region of healing bone.

FIGS. 3A-3F. Bone Volume Fraction Analysis of Alveolar Bone Healing. Bone volume (BV), total volume (TV) and bone volume fraction (BV/TV) are shown for (A-C) experiment 1 using only C57BL/6 WT mice (shown in green; n=4/time point) and (D-F) experiment 2 comparing Ibsp^{-/-} vs. WT mice (shown in red and blue, respectively; n=6/time point) on a mixed 129/CD1 background. (3A) WT BV significantly increased between all time points except between 21 and 56 dpp. (3B) TV was consistent between all time points, with the exception of small (non-significant) changes due to small portions of root tips left behind from procedure; these were manually excluded from analysis. (3C) BV/TV significantly increased between all time points except between 21 and 56 dpp. (3D) BV of WT vs. Ibsp^{-/-} mice was significantly greater at all time points analyzed. (3E) Higher TV was measured in Ibsp^{-/-} vs. WT mice due to enlarged PDL space at 0 dpp, resulting from developmental periodontal defects, as previously demonstrated. (3F) BV/TV was significantly increased in WT vs. Ibsp^{-/-} mice at all time points. Independent samples t-test with the Holm-Šidak correction for multiple comparisons was performed to calculate P-values: *P<0.05; **P<0.01; **** P<0.0001.

FIGS. 4A-4F. Trabecular Analysis of Alveolar Bone Healing. Trabecular bone analyses during socket healing revealed defective healing in Ibsp^{-/-} vs. WT mice (n=4/time point), including (4A) reduced bone volume fraction (BV/TV), (4B) reduced trabecular thickness (Tb.Th) at 21 dpp, (4C) trends towards increased trabecular spacing (Tb.Sp), (4D) decreased trabecular number (Tb.N) at 7 dpp, (4E) decreased bone mineral density (BMD) at all time points, and (4F) decreased tissue mineral density (TMD) at 21 dpp. Independent samples t-test with the Holm-Sidak correction for multiple comparisons was performed to calculate P-values: *P<0.05; **P<0.01.

FIGS. 5A-5B. Osteoclast Quantification during Alveolar Bone Healing. (5A) Histological images showing representative TRAP staining at 7, 21, and 56 dpp in WT and Ibsp^{-/-} mice (n=3-4/time point). (5B) Quantification of osteoclasts (OC) per mm² of bone in healing socket showed no significant differences between genotypes, though Ibsp^{-/-} mice exhibited higher average OC/mm² at all time points. Independent samples t-test with the Holm-Sidák correction for multiple comparisons was performed.

FIGS. 6A-6D. Cell Proliferation during Alveolar Bone Healing. Immunostaining for proliferating nuclear cell antigen (PCNA) allowed quantification of proliferating cells in healing sockets. (6A) Representative images showing PCNA staining and regions of interest (ROI: green dotted outlines) quantified at 7, 21, and 56 dpp in both WT and Ibsp^{-/-} mice (n=3-4/time point). (6B) Normalized density of proliferating cells (cells/mm²) was not significantly different between genotypes, though trended higher in Ibsp^{-/-} mice at 21 and 56 dpp. (6C) Total number of proliferating cells was not significantly different between genotypes, though trended higher in Ibsp^{-/-} mice at 21 and 56 dpp. (6D) Size of ROI did not vary between genotypes at any time point. Independent samples t-test with the Holm-Šídák correction for multiple comparisons was performed.

FIG. 7A-7D. Cell Apoptosis during Alveolar Bone Healing. Cell apoptosis evaluation of healing sockets using the TUNEL assay. (7A) Representative images showing TUNEL-positive cells (green) and regions of interest (ROI; white outlines) quantified at 7 and 21 dpp in both WT and Ibsp^{-/-} mice. Blue staining represents cell nuclei. (7B) Normalized density of apoptotic or TUNEL-positive cells (cells/mm²) was not significantly different between genotypes. (7C) Total number of apoptotic cells was not significantly different between genotypes. (7D) Size of ROI did not vary between genotypes at any time point examined. Independent samples t-test with the Holm-Šídák correction for multiple comparisons was performed.

FIGS. 8A-8B. Principle Component Analysis of Gene Expression Data. Principal component analysis (PCA) plot showing gene expression grouping and variation between WT and Ibsp^{-/-} mice at 0 and 14 dpp. (8A) A score plot for qPCR gene expression PCA WT in blue (light blue for 0 dpp; dark blue for 14 dpp) and Ibsp^{-/-} mice in red/orange (orange for 0 dpp; red for 14 dpp). The area of the bubbles shows the 95% CI. The plot demonstrates major differences between WT and Ibsp^{-/-} mice regardless of time point and smaller differences in gene expression between healing and non-healing (0 dpp vs. 14 dpp) within groups with larger differences in healing for Ibsp^{-/-} vs. WT mice. (8B) A loading plot of same data in (8A) show genes that had the largest impact on differences seen in groupings based on vectors from origin.

FIGS. 9A-9B. Immunostaining of Neutrophils and Macrophages in Alveolar Bone Healing. IHC was performed to identify (9A) neutrophils (by marker NIMP, also known as LY-6C and -6G) and (9B) macrophages (by marker F4/80), as indicated by red-brown signal (n=4/time point). (9A) In positive control tissues, including first molar (M1), periodontal ligament (PDL), and alveolar bone (AB), neutrophils are present mainly at the junctional epithelium (JE). While neutrophils are present in healing AB of both WT and Ibsp^{-/-} mice at 1 day post-procedure (dpp), the cells are cleared at later times and no neutrophils are evident in either genotype at 7-56 dpp. (9B) Control spleen tissues show presence of large numbers of macrophages. Macrophage numbers are elevated in healing AB at early ages of 1 and 7 dpp, then decreases in WT AB by 21 and 56 dpp. Elevated numbers of macrophages persist in Ibsp^{-/-} AB in association with large bone marrow (BM) spaces that include large numbers of macrophages.

FIG. 10. PCA chart of Eigenvalues. Chart shows proportion of variance explained by each principal component (percent in 4^{th} column) and cumulative percent (in 5^{th} column). The majority of variance is explained by the first two components and contribution of components trails off significantly after those components.

FIGS. 11A-11L. Progressive alveolar bone healing in first maxillary molar extraction sockets. (11A) First maxillary molars (M1) were bilaterally extracted from 42-d postnatal (dpn) mice and alveolar bone (AB) healing was studied up to 56 d postprocedure (dpp) (n=4/time point). (11B-11I) Three-dimensional (3D; occlusal and buccal views) and 2-dimensional (2D; transverse and sagittal views) representative images of AB healing at 0, 7, 21, and 56 dpp. In 3D images, red highlights new AB. In 2D images, location of original socket is traced by a yellow dotted line. (11J) Bone volume fraction (BV/TV) progressively increases across all time points. (11K) Bone mineral density (BMD) and (11L) tissue mineral density (TMD) increase at 21 and 56 versus 7 dpp. Brown-Forsythe and Welch analysis of variance was performed to calculate adjusted P values: *P<0.05, **P<0.01, ***P<0.001.

FIGS. 12-12I. Expression of bone sialoprotein (BSP) increases during alveolar bone healing. (12A-12H) Histology showing alveolar bone (AB) healing at 7 d postprocedure (dpp) with location of original socket(S) traced by a yellow dashed line. (12A) Hematoxylin and eosin (H&E) staining shows newly forming AB in the apical portion of S. (12B) Picrosirius red (PR) staining viewed under polarized light microscopy shows collagen organization in new AB. (12C) Ibsp messenger RNA (mRNA) and (12D) BSP protein are highly expressed during AB repair. In situ hybridization or immunohistochemistry highlights key bone markers: (12E) Runx2 (early osteoblasts), (12F) OPN (active osteoblasts), and (12G) Sost (osteocytes). (12H) Tartrate-resistant acid phosphatase (TRAP)—positive multinucleated cells (osteoclasts) show remodeling activity in new AB. (12I) Quantitative polymerase chain reaction for key bone markers (n=6/time point) shows significant upregulation at 14 versus 0 dpp, with the exception of Spp1. Independent samples t test was performed to calculate P values: *P<0.05, **P<0.01, ***P<0.001, ****P<0.0001.

FIG. 13A-13I. Genetic ablation of bone sialoprotein (BSP) reduces alveolar bone healing. First maxillary molars (M1) were bilaterally extracted from 42-d postnatal (dpn) mice, and alveolar bone (AB) healing was studied up to 56 d postprocedure (dpp) (n=4/time point). (13A-13F) Three-dimensional (3D; occlusal and buccal views) and 2-dimensional (2D; transverse and sagittal views) representative images of AB healing at 7, 21, and 56 dpp. In 3D images, red highlights new AB. In 2D images, location of original socket is traced by a yellow dotted line. Compared to WT, Ibsp^{-/-} mice show reduced AB healing throughout the time course. (13G) Bone volume fraction (BV/TV) and (13H) bone mineral density (BMD) are reduced in Ibsp^{-/-} versus WT mice at 7, 21, and 56 dpp. (13I) Tissue mineral density (TMD) is decreased in Ibsp^{-/-} versus WT mice at 21 dpp. Independent samples t test with the Holm-Šídák correction for multiple comparisons was performed to calculate P values: *P<0.05, **P<0.01.

FIGS. 14A-14F. Delayed bone maturation in sockets of Ibsp^{-/-} mice. Histology showing alveolar bone (AB) healing at 7, 21, and 56 d postprocedure (dpp) with location of original socket(S) traced by a yellow dashed line. (14A) Hematoxylin and eosin (H&E) staining shows newly forming AB, initiating in the apical portion of S as woven bone (yellow star) and becoming mature, compact bone (green star) in wild-type (WT) mice between 7 and 56 dpp. Ibsp^{-/-} mice are notable for diminished AB formation, large areas lacking AB formation, persistent woven bone (yellow star), and large marrow spaces (red stars). (14B) Picrosirius red (PR) staining observed with polarized light microscopy shows collagen organization in new AB. Ibsp^{I} and WT mice show similar collagen deposition but persistent woven bone (green stars) and lack of mature, compact bone (yellow stars). High levels of both (14C) Col1a1 and (14D) Alpl messenger RNA (mRNA) decrease over time as new AB matures in WT, but both remain elevated around woven bone in Ibsp^{-/-} mice. (14E) WT mice show exhibit Ibsp mRNA expression in osteoblasts during AB healing. Punctate Ibsp staining in osteoblasts is observed in Ibsp^{-/-} mice due to gene knockout targeted after the signal peptide. (14F) Immunohistochemistry shows robust expression of BSP protein in new AB of WT mice and confirms lack of BSP in Ibsp^{-/-} mice. For histology, n=2-4 mice/genotype/time point were used.

FIGS. 15A-15G. Dysregulated gene expression in alveolar bone of Ibsp^{-/-} mice during healing. Quantitative polymerase chain reaction (qPCR) array was performed to compare alveolar bone healing of Ibsp^{-/-} versus wild-type (WT) mice at 14 d postprocedure (dpp) (n=6/time point). Gene-centric heatmaps of the qPCR array summarize results from 92 target genes are organized into 7 functional groups associated with osteogenesis. Scale is from blue (highest expression in group) to orange (lowest expression in group), with significant results denoted below gene, accompanied by percent increase or decrease in Ibsp^{-/-} versus WT mice. (15A) In the cell signaling group, 11 of 20 genes (55%) are significantly reduced in Ibsp^{-/-} versus WT mice, including Axin2, Gli1, Gli2, Ihh, Itgb1, Itgb5, Mapk1, Nog, Smad3, Smad4, and Wnt11. (15B) The osteoclast biology group shows mixed regulation, with 3 genes downregulated (Ar, Dkk1, and Tnfrsf11b) and 1 upregulated (Ctsk), out of 8 total (38%). (15C) In the osteoblast group, 6 of 14 genes (43%) are reduced, including Bglap, Bmp2, Bmp7, Igf1r, Pth, and Sost. (15D) Changes in mineralization genes are mixed, with 7 of 12 genes (58%) dysregulated, including increased Spp1 and decreased Ank, Bmp3, Enpp1, Ibsp, Mepe, and Phex. (15E) Growth factors were decreased, with 3 of 9 (33%) significantly reduced, including Angpt1, Egf, and Fgf1. (15F) Inflammation-associated genes were increased, with 3 of 9 (33%) genes reduced, including Il1b, Il6, and Il10. (15G) Extracellular matrix genes are downregulated, with 3 of 12 genes (25%) reduced, including Ddr1, Ddr2, and Mmp2. The full list of comparisons for Ibsp^{-/-} versus WT mice at 14 dpp is shown in FIG. 10. Independent samples/test with the Holm-Šídák correction for multiple comparisons was performed to calculate P values: *P<0.05, **P<0.01, ***P<0.001, ****P<0.0001.

FIGS. 16A-16K. BSP and Collagen are Retained in Maxillary Molar Sockets. Histology shows that epithelial healing occurs rapidly after maxillary molar extraction. Compared to (16A) the time of extraction at 0 days post-procedure (dpp), extraction sockets show (16B) closure and presence of granulation tissue by 1 dpp, (16C) epithelialization underway by 2 dpp, and (16D) full thickness oral epithelium present by 7 dpp (16E). Box in panel A shows regions used or imaging in panels B-E. IHC in Ibsp^{-/-} mice demonstrates that at (16F-16H) 1 and (16I-16K) 2 dpp, both collagen gel (COL1A1) and BSP are detectable within healing sockets, as indicated by red-brown reaction.

FIGS. 17A-17D. Exogenous BSP Improves Alveolar Bone Healing in WT and Ibsp Knockout Mice. (17A) Representative 2D images from micro-computed tomography shown in sagittal, coronal, and transverse planes demonstrate alveolar bone healing in WT and Ibsp^{-/-} mice administered collagen, nBSP, or rBSP. (17B) Bone volume fraction (BV/TV) of alveolar bone is increased by nBSP and rBSP in both Ibsp^{-/-} and WT mice, compared to collagen alone controls. (17C) Bone mineral density (BMD) of new bone is increased by nBSP and rBSP in both Ibsp^{-/-} and WT mice, compared to collagen controls. (17D) Tissue mineral density (TMD) of healing bone is not different among treatment groups in Ibsp^{-/-} or WT mice. M1-first maxillary molar; M2=second maxillary molar. ***P<0.001; ****P<0.0001.

FIGS. 18A-18D. Histology of Healing Alveolar Bone in WT and Ibsp Knockout Mice. In healing sockets at 14 dpp, (18A) H&E and (18B) PR staining viewed under polarized light reveals predominantly red fibers with smaller numbers of yellow fibers (18C) COL1A1 IHC confirms the collagen content of the new bone in all sockets. (18D) OPN stains reversal lines in the ECM of healing bone in all experimental groups.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Disclosed are the components to be used to prepare the compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular substituted alginate strand or modified functional moiety is disclosed and discussed and a number of modifications that can be made to a number of molecules including the substituted alginate strand or modified functional moiety are discussed, specifically contemplated is each and every combination and permutation of substituted alginate strand or modified functional moiety and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Methods

Provided herein are methods for promoting alveolar bone healing in a subject in need thereof. These methods can comprise contacting the alveolar bone with a composition comprising a bone sialoprotein.

The term “alveolar bone,” as used herein, refers to the ridge of bone located on the jaw bones which contains the tooth sockets. Alveolar bone is very flexible and constantly remodels to accommodate the changing shape and size of the dental structures it contains. Alveolar bone can be divided into two main parts: a thin layer of compact (radiodense) bone (the ‘cortex’ of alveolus) that lines the alveolus proper, in which Sharpey's fibers insert, that is radiographically termed the lamina dura (lamina dura denta). This area is generally radiographically detectable (but not on computed tomography) as a thin radiodense line in brachydont teeth. Secondly, the main alveolar bone surrounding the lamina dura denta cannot be morphologically differentiated from the main bone of the mandible or maxilla in adult brachydont teeth. The most prominent aspect of the alveolar bone beneath the gingival margin (occlusally) is termed the alveolar crest.

In some embodiments, promoting alveolar bone healing in a subject in need thereof can comprise improving alveolar bone density at a locus in the subject, improving alveolar bone fraction at a locus in the subject, improving alveolar bone fraction at a locus in the subject, or a combination thereof.

In some embodiments, the subject has lost a tooth or had a tooth removed resulting in an empty socket in the alveolar bone. In some of these embodiments, contacting the alveolar bone with the composition comprising the bone sialoprotein can comprise applying the composition comprising the bone sialoprotein to the empty socket (e.g., packing or filling the empty socket with the composition comprising the bone sialoprotein). In some of these embodiments, the method can further comprise suturing gingival tissues to secure the composition comprising the bone sialoprotein within the empty socket. The composition comprising the bone sialoprotein can stimulate the growth of alveolar bone to serve as a foundation for a dental implant at the site of the empty socket.

Also provided herein are methods for performing a tooth extraction in a subject. These methods can comprise extracting a tooth from the subject, resulting in an empty socket in the subject's alveolar bone; packing or filling the empty socket with a composition comprising a bone sialoprotein; and suturing gingival tissues to secure the composition comprising the bone sialoprotein within the empty socket.

Also provided are methods for maintaining alveolar bone health in a subject in need thereof. These methods can comprise contacting the alveolar bone with a composition comprising a bone sialoprotein.

In some embodiments, the subject has lost a tooth or had a tooth removed resulting in an empty socket in the alveolar bone. In some of these embodiments, contacting the alveolar bone with the composition comprising the bone sialoprotein can comprise applying the composition comprising the bone sialoprotein to the empty socket (e.g., packing or filling the empty socket with the composition comprising the bone sialoprotein). In some of these embodiments, the method can further comprise suturing gingival tissues to secure the composition comprising the bone sialoprotein within the empty socket. The composition comprising the bone sialoprotein can maintain sufficient alveolar bone at the empty socket to serve as a foundation for a dental implant at the site of the empty socket.

Also provided are methods for treating periodontal disease in a subject in need thereof. These methods can comprise delivering a composition comprising a bone sialoprotein locally to a site affected by the periodontal disease.

Delivering the composition comprising the bone sialoprotein locally to the site affected by the periodontal disease can comprise placing of the composition comprising the bone sialoprotein at the gum line of the site affected by the periodontal disease.

In some embodiments, the periodontal disease can be in a hard tissue destruction phase. In certain embodiments, the composition comprising the bone sialoprotein can be administered in an effect amount to halt or reverse the hard tissue destruction phase of the periodontal disease.

In some embodiments, the composition comprising the bone sialoprotein can be delivered in an effective amount to improve alveolar bone density at the site affected by the periodontal disease, improve alveolar bone fraction at the site affected by the periodontal disease, improve alveolar bone fraction at the site affected by the periodontal disease, or a combination thereof. In certain embodiments, the composition comprising the bone sialoprotein can be delivered in an effective amount to regenerate cementum at the site affected by the periodontal disease.

Also provided herein are methods for improving integration of a dental implant within the alveolar bone of a subject. These methods can comprise contacting the dental implant, the alveolar bone of the subject, or a combination thereof with a composition comprising a bone sialoprotein. For example, in some embodiments, a surface of the dental implant (e.g., a surface of the dental implant to be placed in contact with the alveolar bone of the implant recipient) can be coated with a composition comprising a bone sialoprotein.

### BSP Compositions

The methods described herein can employ a composition suitable for administration within the oral cavity of the subject. The composition can comprise a bone sialoprotein dissolved or dispersed in a pharmaceutically acceptable carrier.

As used herein the term “bone sialoprotein” is intended to include native bone sialoprotein protein isolated from human or other warm-blooded vertebrates, naturally occurring isoforms of bone sialoprotein protein, recombinant protein produced from bone sialoprotein encoding nucleic acid sequences, and protein fragments/peptides of bone sialoprotein proteins. A bone sialoprotein gene is defined herein to include any nucleic acid sequence encoding for bone sialoprotein, including the native gene sequences isolated from human or other warm-blooded vertebrates, any nucleic acid sequences encoding active fragments of bone sialoprotein protein, or any recombinant derivative thereof.

In certain embodiments, the bone sialoprotein can lack one or more post-translational modifications characteristic of native bone sialoprotein protein isolated from human.

In some embodiments, the bone sialoprotein can be substantially pure. As used herein, the term “substantially pure” is intended to mean purified to at least 90% purity, and preferably to 95% purity, as determined by polyacrylamide gel electrophoresis or amino acid analysis. “Purity” and the like refers to degree of absence of contaminants.

Pharmaceutically acceptable carriers suitable for use in delivering BSP to bone in the oral cavity are known to those skilled in the art. In some embodiments, the pharmaceutically acceptable carrier can comprise a polymer matrix. The polymer matrix can be formed from one or more biocompatible polymers. As used herein, biocompatible means that the polymer is non-toxic, non-mutagenic, and elicits a minimal to moderate inflammatory reaction. In some examples, the biocompatible polymer is also biodegradable and completely degrades in a controlled manner into non-toxic residues. In this embodiment, the polymer matrix can serve as a delivery vehicle for the BSP, concentrating the BSP at a localized site of administration and controlling the release.

A variety of polymers can be used to form a polymer matrix for the purposes of delivering BSP to sites in the oral cavity of a subject, including polyesters, ionomers, poly(amino acids), poly(peptides), proteins, polyvinyl acetate, polyvinyl alcohols polyacrylates, polyorthoesters, polyanhydrides, fibrins, starches, polysaccharides such as alginate, or a combination thereof.

One of the advantages of polyesters in such applications is that they are both biodegradable and biocompatible. Aliphatic polyesters have been widely used in the area of biomaterials for implantable drug delivery devices, sutures, and general tissues supports, after injury or surgery. The polyesters traditionally of greatest interest for localized delivery of biomaterials, are derived from lactide, glycolide, and -caprolactone monomers, with a fairly broad range of degradation profiles accessible through various termonomer combinations. The ester linkages in these aliphatic polyesters are hydrolytically and/or enzymatically labile and render the polymers degradable in aqueous environments. In a certain embodiment, polymers such as polyester anhydrides or ionomers are used. Alternatively, other polymers such as polylactic acid and polyorthoesters are also suitable.

In another embodiment, the polymer matrix comprises collagen (e.g., collagen fibers). Collagen can exhibit bioactive properties and enhance the repair of bone in vivo. Further, collagen can associate with the collagen binding domain of BSP. Accordingly, collagen can function as both as a carrier as well as an active agent within the composition.

Other polymers suitable for use in forming the polymer matrix comprise fibrins, starches, alginate, and hyaluronic acid.

The composition of the polymer used to form the polymeric matrix, as well as the molecular weight and physical properties of the polymer, can be varied according to the application. For example, hydrophobic polyanhydrides can be used where it is desirable to increase the time of degradation. Compounds can be mixed into, or polymerized with the polymer as required for additional strength or other desirable physical properties, using materials known to those skilled in the art from studies involving bone cements. For example, tricalcium phosphate or other ceramic type materials that provide better physical handling properties can be added to the composition.

In some embodiments, the polymer matrix can release the BSP over a period of approximately 1 to 60 days. The polymer can optionally also degrade completely over a period no longer than about sixteen to twenty weeks. Release and degradation times can depend in part upon the polymer used and the bioactive materials to be released. In addition to polymers, various other time-release vehicles are known.

In accordance with one embodiment, the polymeric matrix can comprise polyester ionomers (salts of carboxy-terminated polyesters). The polyester ionomers can exhibit good solubility even at higher molecular weights dictated by implant structural/functional requirements. The polyesters can be prepared from and degrade into naturally occurring metabolites for enhanced biocompatibility. The polyester ionomers can be prepared from the corresponding carboxy-terminated polyesters by neutralization or partial neutralization with biocompatible, pharmaceutically acceptable salt-forming bases.

In an embodiment the polymeric matrix can comprise a biodegradable carboxy-terminated polyester in combination with corresponding ionomers. The physical properties of polyester ionomers can be controlled by the degree of neutralization of the corresponding carboxy-terminated polyesters and to some extent by selection of the neutralizing base. The polyester ionomers can be used alone or in combination with their carboxy-terminated polyester precursor for use as a polymeric matrix.

The use of polyester ionomers as polymeric matrix is described in U.S. Pat. No. 5,668,288, the disclosure of which is incorporated herein by reference. In general, the polyester ionomers can be a divalent residue of a polyester. The polyester can comprise a homopolymer, copolymer, or terpolymer of biocompatible hydroxy acids, for example, lactic acid, glycolic acid, -hydroxy caproic acid, and -hydroxy valeric acid. Alternatively, the polyester can be formed using copolymerization of a polyhydric alcohol and a biocompatible polycarboxylic acid. Most typically, such copolymers can be formed between dihydric alcohols, for example, propylene glycol for biocompatibility and biocompatible dicarboxylic acids.

The compositions described herein can comprise bone sialoprotein in combination with (dissolved or dispersed in) a pharmaceutically acceptable carrier. Optionally, other components can be present in the compositions, including solubilizing agents, filler materials, physiological mineral source for bone formation, osteogenic or other growth factors, antimicrobial agent, biocides, and/or preservatives.

In some embodiments, the bone sialoprotein can be present in the composition at a concentration ranging from about 5 ng/ml to about 500 μg/mL. In some embodiments, the bone sialoprotein can be present in the composition at a concentration ranging from about 1 μg/mL to about 20 μg/mL.

In some embodiments, the composition can further comprise a physiological mineral source for bone formation, such as a calcium phosphate. In some example, the composition can further comprise tricalcium phosphate, hydroxyapatite, gypsum, or a combination thereof. Tricalcium phosphate, hydroxyapatite, gypsum, or other suitable physiological mineral sources can be combined with the compositions to assist in repair of damaged or diseased bone. In some embodiments, a physiological compatible mineral can comprise up to 80% of the BSP composition. Alternatively, the physiological compatible mineral can comprise from about 5% to about 50% of the composition, such as from about 5% to 30% of the composition.

In some embodiments, the composition can further comprise a growth factor, growth factor binding protein, eukaryotic cell. Examples of suitable growth factors include: fibroblast growth factor, parathyroid hormone, osteogenin, transforming growth factor (e.g., TGF-β), bone morphogenetic protein, epidermal growth factor, or platelet-derived growth factor. Examples of growth factor binding proteins include insulin-like growth factor binding proteins (IGFBP's) such as IGFBP 3 and 5. Examples of suitable eukaryotic cells include bone marrow cells, osteoblasts and mesenchymal stem cells.

In some embodiments, the composition can further comprise an osteogenic agent that stimulates or accelerates generation of bone upon implantation into a bone defect site. Examples of osteogenic agents comprise demineralized bone powder, morselized cancellous bone, aspirated bone marrow, bone or cartilage forming cells, and other bone sources.

In some embodiments, the composition can further comprise comprising an antimicrobial agent, such as metronidazole, tobramycin, gentamicin, or vancomycin.

In some embodiments, the composition can further comprise a solubilizing agent, such as dimethyl sulfoxide, Cremphor EL, or one or more surfactants.

In some embodiments, the composition can be a liquid (e.g., a viscous liquid) or gel. In these embodiments, the composition can be formulated to be injectable. The viscosity of the compositions can be adjusted by controlling the water content of the compositions or by the addition of pharmaceutically acceptable fillers or thickening agents known to those skilled in the art. In one embodiment, the composition can include collagen fibers and the viscosity of the composition is controlled by adjusting the pH of the composition to about 6.0 to about 7.5.

In other embodiments, the composition can be a solid. For example, the composition can comprise (or be formed as) a film, a fiber, a filament, a sheet, a thread, a cylindrical implant, a powder, a particulate, an asymmetrically-shaped implant, or a fibrous mesh.

Some particular embodiments of interest include those described in more detail below.

### Alveolar Bone Healing/Alveolar Ridge Preservation

Tooth extractions are necessary for a variety of reasons, including severe tooth decay, tooth fractures, tooth infections, tooth resorption, and severe periodontal disease. Following removal of a tooth, alveolar bone (that part of the mandibular and maxillary bone which surrounds the teeth and forms the tooth sockets) becomes dramatically reduced in height and thickness as the bone is resorbed lacking any mechanical input from occlusion of the teeth. A certain level of bone quantity and quality is required for dental implant placement, and alveolar bone loss can compromise the ability to successfully place implants.

Bone grafting is sometimes performed to try to maintain bone after tooth extraction. Socket bone grafts (also referred to as socket preservation or alveolar ridge preservation) are usually performed at the time of tooth extraction. After the tooth is extracted, the socket is immediately filled with bone grafting material. Bone graft may be composed of allografts (cadaver bone) or different types of xenografts (e.g., deproteinized bovine bone matrix or animal-derived sources of bone). The bone graft is placed in the empty socket and gingival (gums) tissues are sutured to close the wound and allow healing over the next several months. This newly formed bone will provide the foundation for the dental implant that will be placed once healing is complete. Substantial bone loss can still occur with grafting, along with other complications, and some grafts fail and must be repeated.

In the context of alveolar bone healing and alveolar ridge preservation, BSP may be added with another material (e.g., collagen, bone graft, calcium phosphate, or other scaffold or vehicle) into the socket at the time of extraction. The BSP can increase the speed and/or effectiveness of alveolar bone healing.

### Dental Implant Integration

Dental implants are sometimes placed in edentulous regions of the maxillary and mandibular jawbone. Dental implants are medical devices surgically implanted into the jaw to restore a person's ability to chew. Implants provide support for artificial teeth, such as crowns, bridges, or dentures. Sufficient alveolar bone quality and quantity is essential for successful dental implant placement and retention. If bone quality/quantity is not sufficient, bone grafting may need to be performed and several months may be required for bone accumulation. At the time of implant placement, the oral surgeon will make a cut to open the gum and expose the bone. Holes will be drilled into the bone where the dental implant metal post will be placed. Since the post will serve as the tooth root, it is implanted deep into the bone and bone cells will respond to osseointegrate the implant. Some implants fail due to insufficient bone or development of peri-implantitis, an inflammatory condition that threatens to cause implant failure. Thus, there is interest in the use of growth factors at the time of dental implant placement, to improve bone healing and implant integration and stability.

In the context of dental implant placement, BSP compositions may be added to the bone surrounding the implant, or the implant may be coated with a BSP composition. The BSP composition can increase osseointegration of the implant, resulting in improved retention.

### Periodontal Disease

The periodontium is a complex structure that contains at least six distinct tissue types, including the gingival epithelium, the gingival connective tissue, the periodontal ligament (PDL), the tooth root surface cementum, the alveolar bone, and corresponding vasculature. The periodontium exhibits a typical “layer by layer” (LBL) structure comprising cementum, alveolar bone and periodontal ligament (PDL). The cementum occurs as a thin acellular layer around the tooth root neck, with thicker cellular cementum covering the lower part of the tooth root up to the apex. The PDL consists of highly organized fibers, which are perpendicularly inserted into the cementum coated tooth root and adjoining the alveolar bone, where their ends (Sharpey's fibers) insert into the mineralized tissues to stabilize the tooth root, transmit occlusal forces, and provide sensory function. PDL fibers connect the cementum on the tooth root surface to the alveolar bone and fix the tooth in the alveolar socket to attenuate occlusal stresses.

Periodontal disease or periodontitis is a chronic inflammatory disease that begins with a period of inflammation of the supportive tissues of the teeth and then progresses. It is a common cause of receding gums that can lead to tooth loss and other serious health complications. All of these tissues are affected during chronic inflammation.

Periodontitis is initiated by an imbalance that causes the accumulation of pathogenic bacteria and their lipopolysaccharides. The destruction of the supporting tissues of the tooth in periodontitis is mainly due to an exacerbated immune response of the host in susceptible individuals, which prevents the acute inflammation from being resolved. In these cases, the accumulation of bacteria in the gingival sulcus causes the migration of polymorphonuclear neutrophils (PMNs) and monocytes. These cells, together with those of the gingival epithelium, secrete cytokines such as interleukin IL-1β, IL-6 tumor necrosis factor-alpha (TNF-α), and adhesion molecules such as endoglin and intercellular adhesion molecule 1 (ICAM-1), which increase the adhesion of PMNs and monocytes to endothelial cells and increase the permeability of the gingival capillaries, which leads to the accumulation of leukocytes in the infection zone. This allows macrophages that have arrived at the area of the lesion to produce prostaglandin 2 [PGE2]; high levels of this molecule and IL-1β increase the binding of PMNs and monocytes to endothelial cells, exacerbating inflammation, which, together with IL-6 and TNF-α, induce osteoclasts to activate and reabsorb the alveolar bone. Local capillaries release a large amount of serum as a result of the release of histamine and complement molecules, which leads to increased vascular permeability. This serum is converted into a tissue fluid that contains inflammatory peptides (antibodies, complement, and other agents that mediate the body's defenses) that are carried into the gingival sulcus. Increased gingival fluid causes the tissues and the amount of gingival crevicular fluid to increase in volume. Macrophages and neutrophils in the infection area contain enzymes (e.g., nicotinamide adenine dinucleotide phosphate (NADPH) oxidase and myeloperoxidase) that produce reactive oxygen species (ROS) to eliminate pathogens. Under normal conditions, antioxidant mechanisms protect the tissues from damage mediated by ROS. However, if the body's antioxidant capacity is insufficient against ROS, oxidative stress occurs that damages the hard and soft tissues of the periodontium. In addition, excessive release of pro-inflammatory cytokines is stimulated through activation of nuclear factor κB (NFκB) and the production of PGE2, which is related to bone resorption. If this situation is sustained, the epithelial adhesion is destroyed and the alveolar crest, which is an extension of both the mandible and the maxilla and holds the tooth sockets, loses its height, which translates clinically into dental mobility and formation of periodontal pockets, causing the accumulation of more bacteria that increase the problem, thereby completely destroying the periodontal ligament connecting the cementum of the teeth to the gingivae and alveolar bone; the alveolar bone becomes atrophied, and the tooth is lost.

Periodontitis is first treated by prophylactic measures to remove the infection. Scaling and root planing is a deep cleaning below the gumline used to treat periodontitis. However, cleaning measures do not promote repair or regeneration of periodontal tissues lost to disease. Current approaches to promote periodontal regeneration are unpredictable and lead to variable results, particularly in cementum, which is notoriously difficult to repair. This is, in part, due to lack of insights into periodontal development and biological factors that could be employed to recapitulate periodontal development in disease states. Bioactive factors may be used to promote periodontal repair. One such biological factor is Emdogain®, derived from developing porcine tooth germs. If the disease site is accessible from the surface, Emdogain® is applied to the root surface with a syringe, followed by suturing. If the disease site is deeper, anesthetic is applied and the periodontist will make a small incision to expose the root surface, creating a flap of tissue. Emdogain® can then be applied to the root surface. Emdogain® results in marginal periodontal repair in many cases.

In the context of periodontal disease, BSP may be added as a gel or with a scaffold or delivery system to the superficial or deep root surface to promote repair of cementum and alveolar bone, which can promote improved periodontal attachment and function, and tooth retention.

### Traumatic and Other Defects

Traumatic defects to jawbone can occur from motor vehicle and sports accidents, surgeries to remove tumors, or acquired destructive conditions, sometimes resulting in jaw fracture, tooth loss, and critical size defects that will not heal on their own. Current methods to treat these types of bone defects include surgical procedures and use of large scaffolds to deliver cells or biological factors.

In the context of repair of traumatic defects, BSP may be added as a gel or with a scaffold or delivery system to the defect area to promote new bone formation.

By way of non-limiting illustration, examples of certain embodiments of the present disclosure are given below.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. Efforts have been made to ensure accuracy with respect to numbers (e.g., amounts, temperature, etc.), but some errors and deviations should be accounted for. Unless indicated otherwise, parts are parts by weight, temperature is in °C or is at ambient temperature, and pressure is at or near atmospheric.

### Example 1. Bone Sialoprotein is Critical for Alveolar Bone Healing in Mice

### Overview

Bone sialoprotein (BSP) is an extracellular matrix (ECM) protein associated with mineralized tissues, particularly bone and cementum. BSP includes functional domains implicated in collagen binding, hydroxyapatite nucleation, and cell signaling, although its function(s) in osteoblast and osteoclast differentiation and function remain incompletely understood. Genetic ablation of BSP in Ibsp knockout (Ibsp^{-/-}) mice results in developmental bone mineralization and remodeling defects, with alveolar bone more severely affected than the femurs and tibias of the postcranial skeleton. The role of BSP in alveolar bone healing has not been studied. We hypothesized that BSP ablation would cause defective alveolar bone healing. We employed a maxillary first molar extraction socket healing model in 42-d postnatal Ibsp^{-/-} and wild-type (WT) control mice. Tissues were collected at 0, 7, 14, 21, and 56 d post procedure (dpp) for analysis by micro-computed tomography (microCT), histology, in situ hybridization (ISH), immunohistochemistry (IHC), and quantitative polymerase chain reaction (qPCR) array. Alveolar bone healing progressed in WT mice with increasing bone volume fraction (BV/TV), bone mineral density (BMD), and tissue mineral density (TMD), transitioning from woven to mature bone from 7 to 56 dpp. Ibsp messenger RNA (mRNA) and BSP protein were strongly expressed during alveolar bone healing in parallel with other osteogenic markers. Compared to WT, Ibsp^{-/-} mice exhibited 50% to 70% reduced BV/TV and BMD at all time points, 7% reduced TMD at 21 dpp, abnormally increased Col1a1 and Alpl mRNA expression, and persistent presence of woven bone and increased bone marrow in healing sockets. qPCR revealed substantially dysregulated gene expression in alveolar bone of Ibsp^{-/-} versus WT mice, with significantly disrupted expression of 45% of tested genes in functional groups, including markers for osteoblasts, osteoclasts, mineralization, ECM, cell signaling, and inflammation. We conclude that BSP is a critical and nonredundant factor for alveolar bone healing, and its absence disrupts multiple major pathways involved in appropriate healing.

### Introduction

Bone sialoprotein (BSP) is a noncollagenous extracellular matrix (ECM) protein associated with mineralized tissues, including alveolar bone and cementum of the periodontal complex (Ganss et al. 1999; Goldberg and Hunter 2012). BSP includes 3 highly conserved functional domains. The N-terminal collagen-binding domain promotes BSP-collagen interactions. Two (or more, depending on species) polyglutamic acid (polyE) domains contribute to hydroxyapatite (HA) crystal nucleation and/or growth (Hunter and Goldberg 1994; Goldberg et al. 1996; Tye et al. 2003), which can also be influenced by BSP serine phosphorylation and O- and N-linked glycosylation (Miwa et al. 2010; Xu et al. 2017). The third functional domain is an arginine-glycine-aspartic acid (RGD) integrin-binding motif associated with osteoblast gene expression, adhesion, and migration (Gordon et al. 2007, 2009). Although in vitro experiments have informed about BSP functional roles associated with these domains, in vivo function(s) of BSP in bone biology remain incompletely understood.

Genetic ablation of BSP in Ibsp knockout (Ibsp^{-/-}) mice leads to reduced skeletal size, defective osteoblast differentiation, delayed long bone mineralization, reduced osteoclast activity, and abnormal bone remodeling (Malaval et al. 2008; Boudiffa et al. 2010; Holm et al. 2015). Impaired bone healing was found in challenge models, including surgically created cortical bone defects and bone marrow ablation in Ibsp^{-/-} mouse femurs (Malaval et al. 2009; Wade-Gueye et al. 2012). Loss of BSP caused alveolar bone hypomineralization and acellular cementum hypoplasia that contributed to periodontal breakdown (Foster et al. 2013; Foster et al. 2015; Soenjaya et al. 2015; Ao et al. 2017). Excessive accumulation of osteoid in alveolar bone of Ibsp^{-/-} mice suggested a more critical role for BSP in mineralization in this region, possibly associated with neural crest-derived osteoprogenitor cells or intramembranous ossification.

We adapted a mouse molar extraction and alveolar bone healing model to challenge Ibsp^{-/-} mice and better understand the functions of BSP in alveolar bone biology, particularly alveolar bone healing following tooth extraction. We hypothesized mice lacking BSP would exhibit delayed or defective alveolar bone healing. A multimodal approach was used to analyze healing and define mechanisms underlying BSP functions in the craniofacial region.

### Materials and Methods

Mice. Animal studies were approved by the Ohio State University Institutional Animal Care and Use Committee and followed ARRIVE guidelines 2.0. Ibsp^{-/-} mice have been previously characterized (Foster et al. 2013; Foster et al. 2015). Two sets of alveolar bone-healing experiments were performed based on previous studies (Pei et al. 2017; Yuan et al. 2018). In the first experiment, procedure optimization, healing time points, and gene/protein expression were established using 42-d postnatal (dpn) C57BL/6J mice (n=4 mice/time point, 2 males/2 females). In the second experiment, alveolar bone healing was compared in 42 dpn Ibsp^{-/-} versus wild-type (WT; Ibsp^{+/+}) littermate control mice on a mixed 129/CD1 background (n=6 mice/genotype/time point, 3 males/3 females). Molar extraction was performed as described below. Tissues were harvested at 0, 7, 14, 21, and 56 d post procedure (dpp) (n=4 mice/genotype/time point). Left maxillae were fixed in 10% formalin for 24 h for micro-computed tomography (microCT) and histology. Healing molar sockets were dissected from the right maxillae for RNA collection.

MicroCT. MicroCT analysis was performed as described previously (Chavez et al. 2021). Maxillae were oriented using a sample registration approach, and region of interest (ROI) was defined by creating an average M1 socket volume, as described below. Tissue above 650 mg/cm³ HA was defined as bone and used to calculate bone volume fraction (BV/TV), bone mineral density (BMD), and tissue mineral density (TMD).

Histology. Tissues for histology were decalcified in an acetic acid/formalin/sodium chloride solution and paraffin embedded for 6-μm sagittal sections. Hematoxylin and eosin (H&E), picrosirius red, and tartrate-resistant acid phosphatase (TRAP) staining and quantification, in situ hybridization (ISH), immunohistochemistry (IHC), and terminal deoxynucleotidyl transferase dUTP nick end labeling (TUNEL) are described below.

Quantitative Polymerase Chain Reaction Array. RNA was collected from healing alveolar bone as previously described (Zhang et al. 2020) and as detailed below. Quantitative polymerase chain reaction (qPCR) arrays included custom TaqMan probes loaded into 96-well plates targeting 92 genes in 7 functional groups and including 4 endogenous housekeeping controls (18S, Gapdh, Ppib, and Gusb).

Statistical Analyses. Mean±standard deviation (SD) are shown in graphs (*P<0.05; **P<0.01; ***P≤0.001; ****P≤0.0001). Analyses were performed with GraphPad Prism v9.0.2 (GraphPad Software) for microCT measurements and ExpressionSuite Software v1.2 (Thermo Fisher Scientific) for qPCR. Additional details are included below.

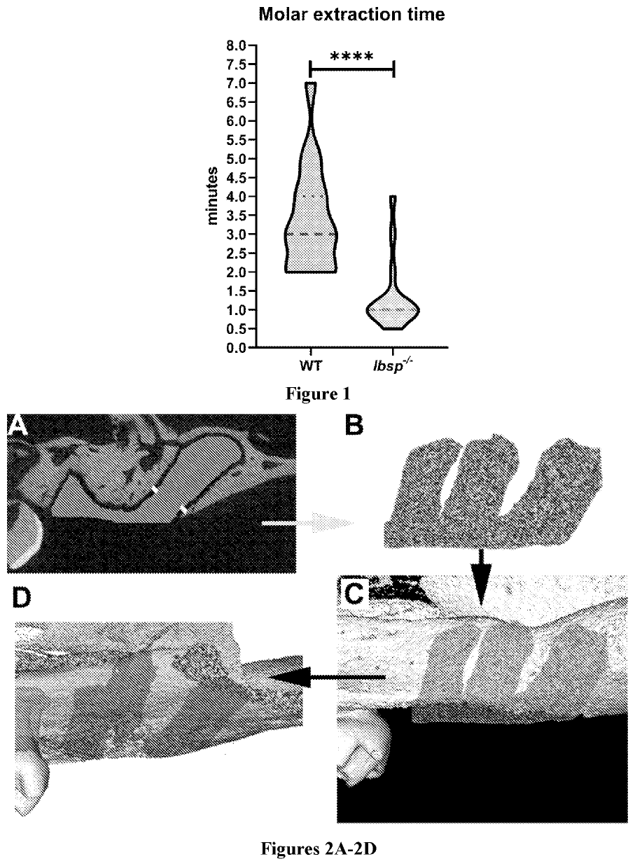

Molar Extraction. Animal studies were approved by the Ohio State University Institutional Animal Care and Use Committee (protocol #2015A00000078-R2) and followed ARRIVE guidelines 2.0. Two sets of alveolar bone healing experiments were performed. In the first experiment, procedure optimization, healing time points, and gene/protein expression were established using C57BL/6J mice (n=4/time point). In the second experiment, alveolar bone healing was compared in Ibsp^{-/-} and wild-type (WT) littermate control mice (n=6/genotype/time point) on a mixed CD1/129 background. Ibsp^{-/-} mice have been previously characterized (Ao et al. 2017; Foster et al. 2015; Foster et al. 2013). For both sets of experiments, mice at 42 days postnatal (dpn) were anesthetized by nasal isoflurano and bilateral extraction of maxillary first molars (M1) was accomplished using a #2 dental explorer. Ibsp^{-/-} mice have reduced periodontal attachment of molars due to defective acellular cementum (Foster et al. 2015; Foster et al. 2013). We noted that anesthetized and positioned WT mice required an average of 3 minutes to extract each M1, while in comparison, teeth were noticeably mobile in Ibsp^{-/-} mice and required less than one minute to extract (FIG. 1). Mice received 2 mg/kg meloxicam and were provided hydrogel and softened food for 3 days post procedure (dpp), and weight was monitored during healing.

Criteria for animal removal from the study included 20% or more weight loss from baseline body weight, body condition scoring indicating illness, or infection of surgical wounds deemed untreatable by veterinary staff. Over the course of the study, n=94 mice underwent extraction, and none were removed from the study due to postsurgical surgical complications. Weight loss of approximately 4% was typically observed at 1 dpp. Mice quickly recovered to starting weight or above by 3 dpp. After regaining consciousness after sedation, mice returned to normal behavior of eating, drinking, and grooming within minutes. Grooming following extraction procedures introduced some complicating variables encountered in analysis of alveolar bone healing; hairs became lodged in sockets of some mice. Bone formation was prevented in the immediate area around lodged hairs, and this inhibition persisted throughout the entire experiment. To reduce hair in sockets, we attempted to use mouse head cones for 1 dpp, but their placement could not be maintained, and this approach was abandoned. Despite the increased variability of healing around embedded hair, results remained consistent within and between groups as hair was observed in both Ibsp^{-/-} and WT mice.

Tissues were harvested at study endpoints of 7, 14, 21, and 56 dpp. Left maxillae were barvested and fixed at room temperature in 10% formalin for 24 hours for micro-computed tomography and histology. Healing molar sockets were dissected from the right maxillae for RNA collection.

Additional Information Regarding Micro-computed Tomography (MicroCT). Samples were scanned in a μCT 50 (Scanco Medical, Bassersdorf, Switzerland) at 70 kVp, 76 μA, 0.5 mm Al filter, with 900-ms integration and 6-μm voxel dimension. DICOM files were created from raw data, exported, and calibrated to a standard curve calculated from five known densities of hydroxyapatite (mg/cm³ HA). Reconstructed images were loaded and analyzed using Analyze 14.0 (AnalyzeDirect, Overland Park, KS) as previously described (Chavez et al. 2021; Chavez et al. 2019; Foster et al. 2018; Lira Dos Santos et al. 2021; Zhang et al. 2020a).

Maxillae were oriented to a standard orientation and were registered to a standard image to automate orientation and ensure very close alignment between samples for analysis of alveolar bone healing in the extraction sockets. To define the region of interest (ROI) for measurement of bone healing, sockets of n=6 WT and Ibsp^{-/-} mice at 0 days post-procedure (dpp) were traced independently and used to generate an average socket map. This average socket region was then eroded by 120 μm to minimize inclusion of preexisting lamina dura and localize the ROT entirely within the empty socket where only new bone formation occurs after molar extraction (FIG. 2). This ROI was mapped onto oriented maxillae and all tissue within the ROI above 650 mg/cm³ HA was defined as bone. This traced ROI was used to calculate bone volume fraction (BV/TV), bone mineral density (BMD), and tissue mineral density (TMD) for new bone. For trabecular bone analysis of healing alveolar bone, the ROI described above was further subdivided to include only the mesial root. This ROI was then analyzed using the bone module add-on in Analyze 14.0 (AnalyzeDirect, Overland Park, KS) to perform recommended analysis of trabecular bone parameters as described (Bouxsein et al. 2010).

Additional Information Regarding Histology. After microCT scanning, left maxillae were demineralized in an acetic acid/formalin/sodium chloride (AFS) solution, paraffin embedded, sectioned at 6 μm. Histological stains included hematoxylin and eosin (H&E) and picrosirius red, using standard protocols previously described (Foster 2012).

In situ hybridization (ISH) was performed using antisense probes for/hsp. RUNX family transcription factor 2 (Runx2), sclerostin (Sost), tissue-nonspecific alkaline phosphatase (Aipl). and collagen type 1 alpha 1 chain (Col1a1) visualized with fast red dye (Advanced Cell Diagnostics, Newark, CA), and counterstained with hematoxylin as previously described (Foster et al. 2018; Zhang et al. 2020a).

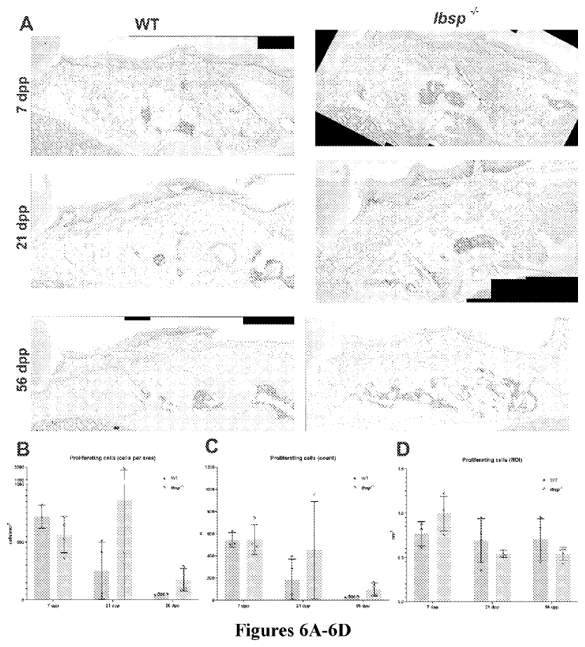

Immunohistochemistry (IHC) was performed with an avidin-biotinylated peroxidase kit with 3-amino-9-ethylcarbazole substrate (Vector Labs, Burlingame, CA), Primary antibodies used were as follows: rabbit polyclonal anti-bone sialoprotein IgG (BSP; courtesy of Dr. Renny Franceschi, University of Michigan) (Foster et al. 2015, Foster et al. 2013); rabbit polyclonal anti-mouse osteopontin IgG (LF-175; OPN, courtesy of Dr. Larry Fisher) (Foster et al. 2018); and rabbit polyclonal anti-proliferating nuclear cell antigen (PCNA; AbCam ab 18197) (Chavez et al. 2019). For measurement of proliferating cells, the socket area was traced (see FIG. 6), and PCNA-positive cells within this ROI were counted using Analyze 14.0 (AnalyzeDirect, Overland Park, KS). These results were reported as total cell numbers, ROI size, and normalized numbers of proliferating cells per area.

Tartrate resistant acid phosphatase (TRAP) staining was performed as described in manufacturer's protocol (Sigma-Aldrich Chemicals, St. Louis, MO). TRAP-positive cells were quantified and normalized to bone surface perimeter with healing sockets using ImageJ (version 1.52q, National Institutes of Health, Bethesda, Maryland, USA) (Foster et al. 2015).

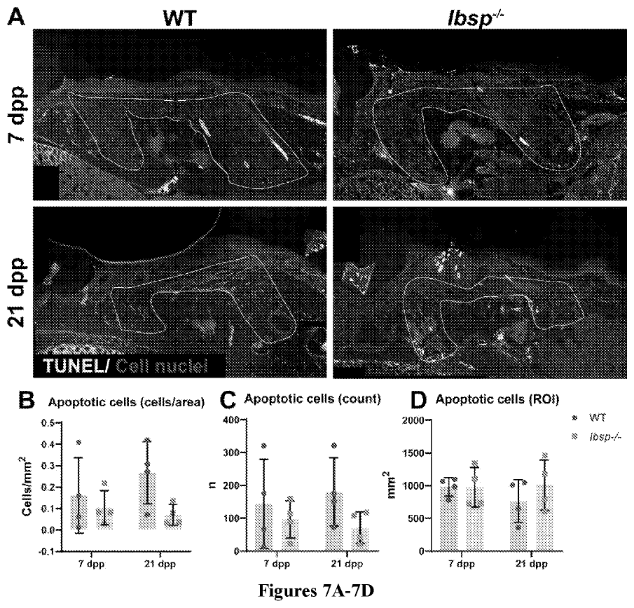

Terminal deoxynucleotidyl transferase dUTP nick end labeling (TUNEL) staining was performed to identify cells undergoing apoptosis with Alexa fluor 488 dye using Click-IT Plus TUNEL assay kit, according to manufacturer's instructions (Life Technologies, Carlsbad, CA). Cell nuclei were counterstained with Hoechst 33342 (blue staining). Images of healing sockets were captured at a 100× magnification using a fluorescent microscope (Zeiss Axio Imager.Z2). TUNEL-positive cells (apoptotic) with green-stained nuclei were quantified and normalized per a defined ROI within healing sockets (avoiding epithelia and lamina propria) at 7 and 21 dpp (see FIG. 7). Quantification of positive cells and ROI area measurements were performed using ImageJ (version 1.52q, National Institutes of Health, Bethesda, Maryland, USA).

Quantitative PCR (qPCR). Right maxillae were harvested for total RNA. Immediately following euthanasia, gingiva and soft tissues were carefully removed and bone was isolated in the region stretching from the rise mesial to M1 to the region mesial to the second molar (M2). The zygomatic arch was removed, leaving the socket of M1 and the immediate adjacent supporting bone. This tissue was placed into 50 μl of TRIzol (Invitrogen, Carlsbad, CA) on ice and pulverized by polypropylene mortar and microtube pestle immersed in liquid nitrogen. An additional 950 μl of TRIzol was added and standard RNA isolation protocol was performed using Phasemaker tubes (Invitrogen, Carlsbad, CA). Total RNA quantity and quality were measured using a NanoDrop spectrophotometer (ThermoFisher Scientific, Waltham, MA) and samples were stored at −80 °C. Reverse transcription was performed using 10 μl of RNA and Superscript IV VILO with ezDNase as directed by the manufacturer (ThermoFisher Scientific). cDNA was then diluted to 10 ng/reaction. Custom TaqMan Probe 96 well plates were designed to include probes/primers for 92 genes organized into 7 functional groups, and 4 endogenous housekeeping controls for normalization (188, Gapdh, Ppib, and Gusb). Target genes are listed in Table 1. qPCR was run in Quantstudio 3 qPCR real-time thermocycler using TaqMan Fast Advanced Master mix following the standard protocol supplied by the manufacturer (ThermoFisher Scientific). Analysis of samples was performed in ExpressionSuite Software v1.2 (ThermoFisher Scientific).

Out of the 92 target genes tested, 9 genes failed to amplify, indicating lack of expression or expression levels below detection limits. These genes were not analyzed further and do not appear in the results.

Additional Information Regarding Statistical Analysis. Mean #standard deviation (SD) are shown in graphs (* P<0.05; ** P<0.01; *** P<0.001; **** P<0.0001). Analyses for microCT measurements were performed using GraphPad Prism v9.0.2 (San Diego, CA). For the first experiment, Brown-Forsythe and Welch ANOVA was performed to compare bone parameters of WT mice at 0, 7, 21, and 56 dpp (n=6/group). For the qPCR experiment, multiple independent samples t-tests with the Holm-Sidak method for correction for multiple comparisons were performed to compare WT and Ibsp^{-/-} mice at each time point (n=6/group). Assumptions of statistical tests were confirmed prior to analysis.

Power analysis was used to estimate animal numbers used. Briefly, for α=0.05 (type I error rate) and power (1−β)=0.8, assuming sampling ratio of 1 (same number of animals/group) and standard deviation of 5-6%, a sample size of 4-6 animals/group would allow for detection of 10% difference as a statistically significant difference. This sample size is consistent with our previous studies on Ibsp^{-/-} mice (Ao et al. 2017; Foster et al. 2015; Foster et al. 2013; Soenjaya et al. 2015) and many other reports on changes in skeletal and dental tissue development and regeneration.

### Results

Bone Sialoprotein Expression Increases during Alveolar Bone Healing. To study the functional importance of BSP during alveolar bone healing, we adapted a mouse model of first maxillary molar (M1) extraction socket healing (FIG. 11A). In the first experiment, procedure optimization, healing time points, and gene/protein expression were analyzed after bilateral M1 extraction from 42 dpn WT mice. MicroCT tracked progressive alveolar bone healing at 7, 21, and 56 dpp, compared to 0 dpp (FIG. 11B-11I). New alveolar bone filled M1 sockets by 21 dpp, while bone structure matured by 56 dpp. Quantitative analysis confirmed the most rapidly increasing bone formation between 7 and 21 dpp (FIG. 11J-11L), with BV/TV increasing 900% (P<0.01), BMD increasing 400% (P<0.01), and TMD increasing 15% (P<0.05). Changes between 21 and 56 dpp were modest and not significant for all parameters.

Histology at 7 dpp and qPCR at 14 dpp were employed to characterize bone healing during this dynamic stage. H&E and picrosirius red staining showed collagenous matrix deposition consistent with woven bone formation initiating in apical sockets (FIG. 12A, 12B). ISH revealed intense expression of Ibsp messenger RNA (mRNA) along new bone surfaces and IHC showed localization of BSP protein in new bone (FIG. 12C, 12D). Other bone markers were confirmed, including Runx2 and OPN (osteoblasts), Sost (osteocytes), and TRAP (osteoclasts) (FIG. 12E-12H). qPCR showed significant upregulation of osteo-genic markers at 14 versus 0 dpp, including Sp7, Runx2, Ibsp, Dmp1, Alpl, Bglap, and Col1a1 (P<0.05-0.0001), with Spp1 trending higher (FIG. 12I).

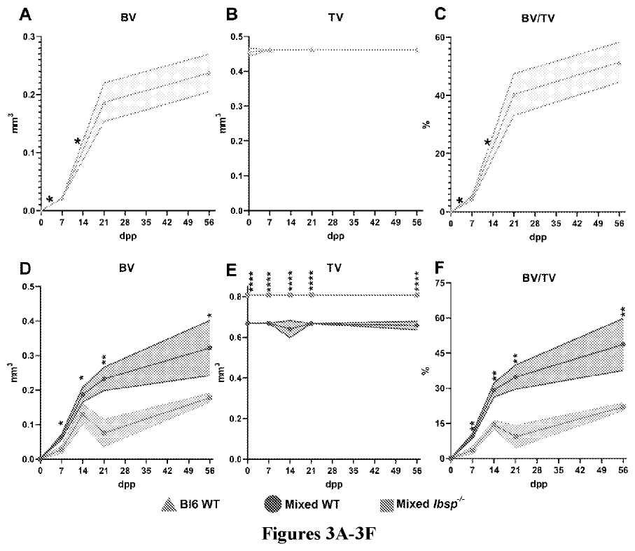

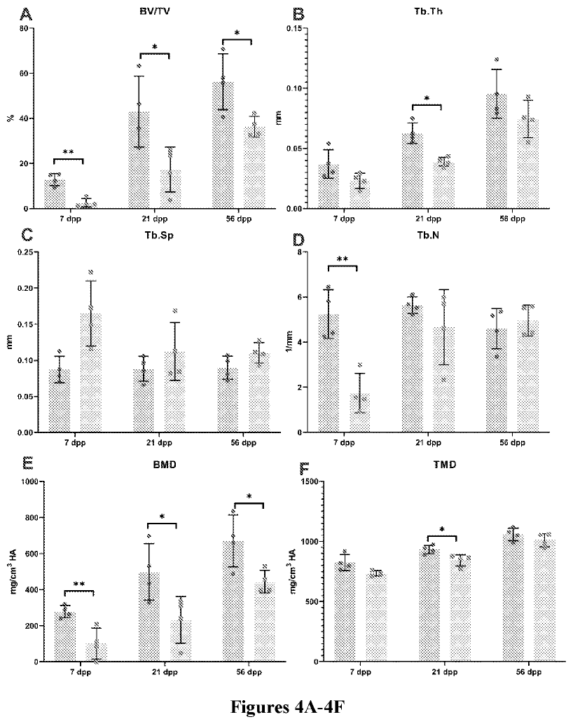

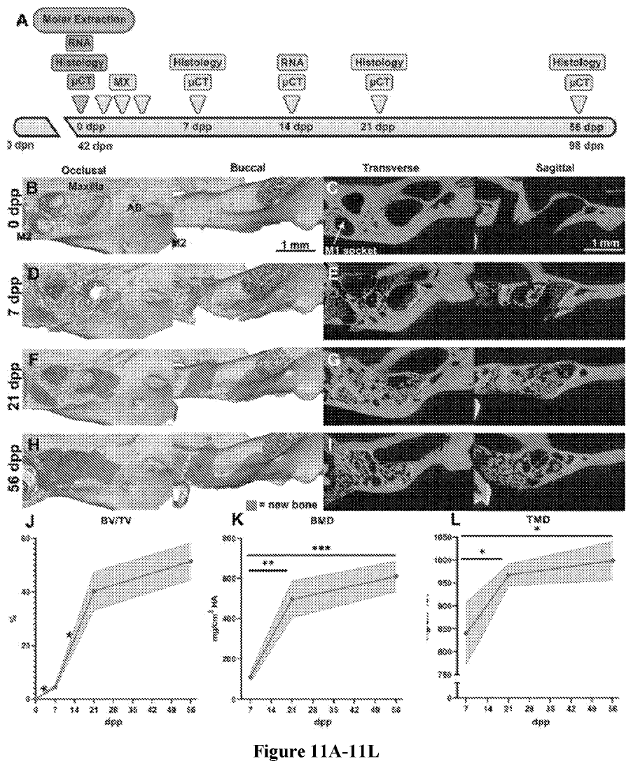

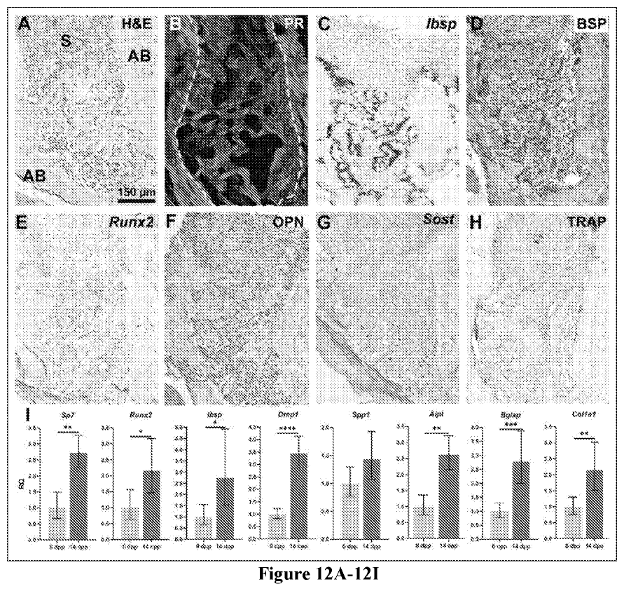

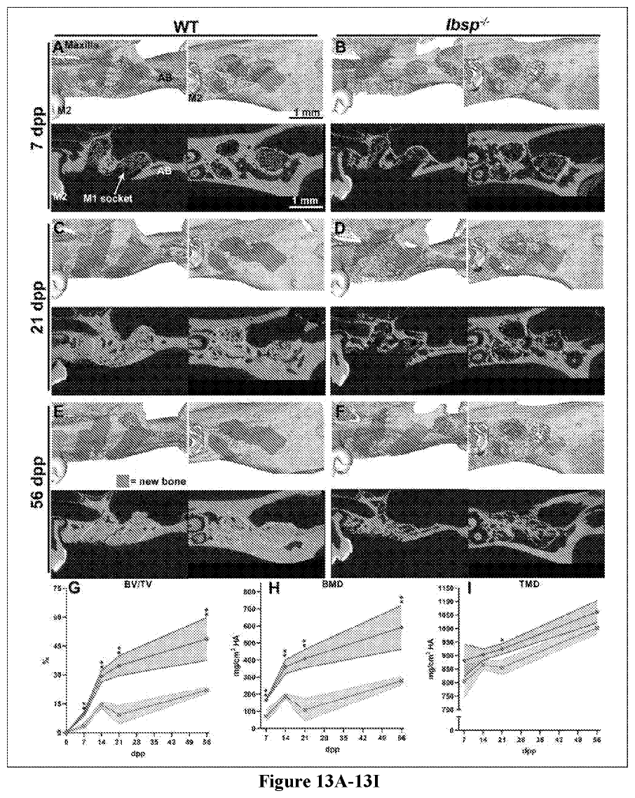

Genetic Ablation of BSP Reduces Alveolar Bone Healing. In the second experiment, alveolar bone healing was compared in Ibsp^{-/-} versus WT mice. Although there was evidence of bone healing in Ibsp^{-/-} M1 sockets, new bone formation was reduced compared to WT mice over the entire time course (FIG. 13A-13F). In Ibsp^{-/-} versus WT mice, BV/TV was reduced 50% to 70% between 7 and 56 dpp (P<0.01 for all comparisons) (FIG. 13G and FIG. 3A-3F). BMD was decreased by 50% to 70% in Ibsp^{-/-} versus WT mice at the same time points (P<0.01 for all comparisons) (FIG. 13H). TMD was reduced 7% in Ibsp^{-/-} versus WT mice at 21 dpp (P<0.01), and other time points trended lower as well (FIG. 13I). Trabecular bone analysis revealed similar deficits in Ibsp^{-/-} versus WT bone healing (FIG. 14A-14F).

Histology provided insights into alveolar bone healing. H&E staining showed progressive bone deposition in both genotypes from 7 to 56 dpp (FIG. 14A). WT sockets were partially filled with woven bone at 7 dpp, and the quantity and maturity of bone increased until 56 dpp. In contrast, Ibsp^{-/-} mice showed reduced quantities of new bone. Picrosirius red staining observed with polarized light microscopy revealed the expected transition in WT mice from woven bone (7 dpp) to a more compact bone structure (21 and 56 dpp) (FIG. 14B). Ibsp^{-/-} mice showed similar collagen deposition, but throughout the course of the study, new bone retained the appearance of woven, disorganized bone, with persistent large marrow spaces. High levels of Col1a1 mRNA were observed by ISH in both WT and Ibsp^{-/-} mice at early time points in cells on new bone surfaces (presumptive osteoblasts) (FIG. 14C). While Col1a1 in WT mouse sockets decreased over time as bone matured, levels in Ibsp^{-/-} woven bone remained abnormally elevated. Alpl mRNA showed a similar pattern, where expression remained unusually elevated in Ibsp^{I} versus WT mice (FIG. 14D). In WT mice, Ibsp mRNA was highly expressed in the entire healing socket at 7 dpp and in osteoblasts at 21 and 56 dpp (FIG. 14E). Some punctate Ibsp mRNA staining in osteoblasts was observed by ISH in Ibsp^{-/-} mice due to gene knockout targeted downstream of the signal peptide. IHC showed localization of BSP protein in new WT bone and lack of BSP in Ibsp^{-/-} mice (FIG. 14F).

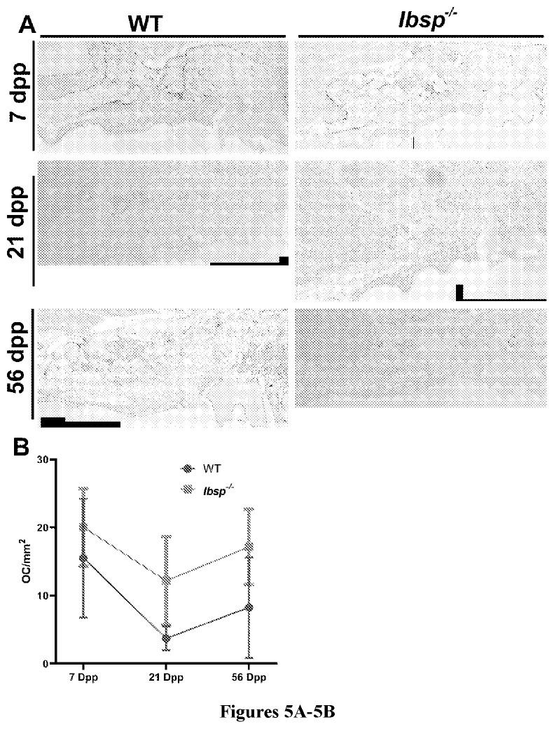

TRAP staining indicated nonsignificant trends toward increased numbers of osteoclast-like cells in Ibsp^{-/-} versus WT mouse sockets at all time points (FIG. 5A-5B). No significant differences in proliferation were noted between genotypes, although average density of proliferating cells trended higher in Ibsp^{-/-} versus WT mice at 21 and 56 dpp (FIG. 6A-6D). No differences were noted in apoptosis between genotypes (FIG. 7A-7D).

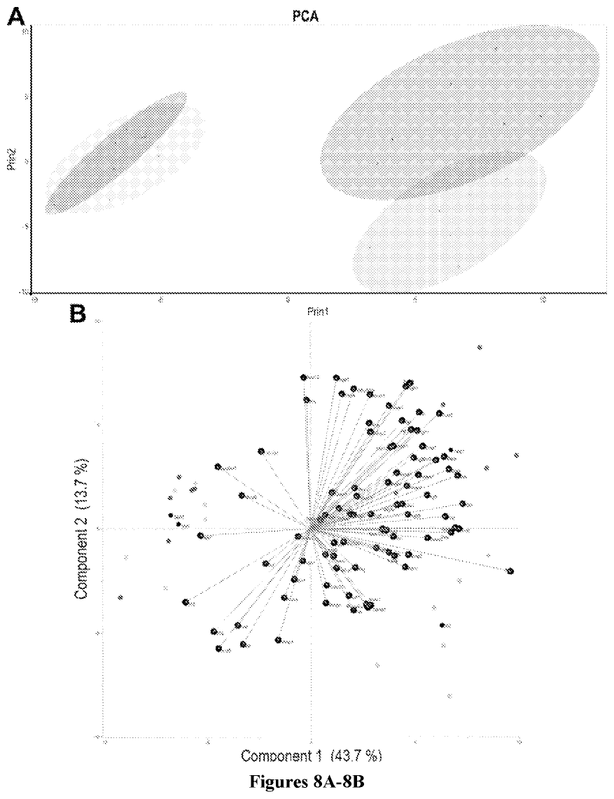

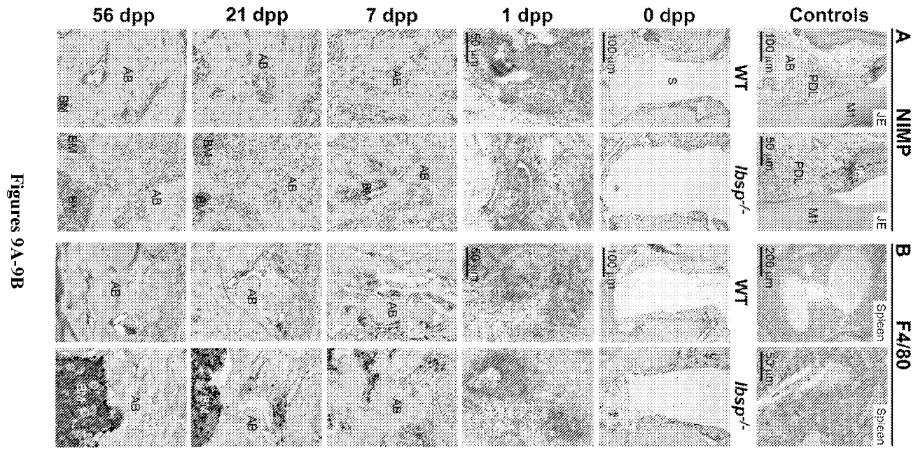

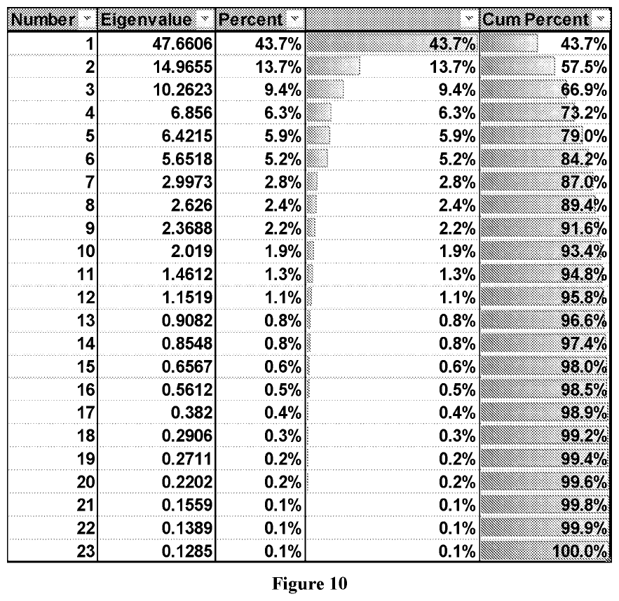

Dysregulated Gene Expression in Alveolar Bone of Ibsp^{-/-} Mice during Healing. To provide insights into bone-healing defects in Ibsp^{-/-} mice, we performed qPCR array for 92 target genes organized into 7 functional groups. Principal component analysis (PCA) of qPCR results indicated dramatic baseline differences (i.e., at 0 dpp) between Ibsp^{-/-} and WT mice (FIG. 8A-8B). During healing, gene expression in WT bone did not substantially differ from baseline; however, Ibsp^{-/-} bone exhibited a distinct shift in gene expression. PCA component 1 accounted for 43.7% of variation, while component 2 accounted for an additional 13.7%, accounting for the majority (57.5%) of variation (FIG. 10).

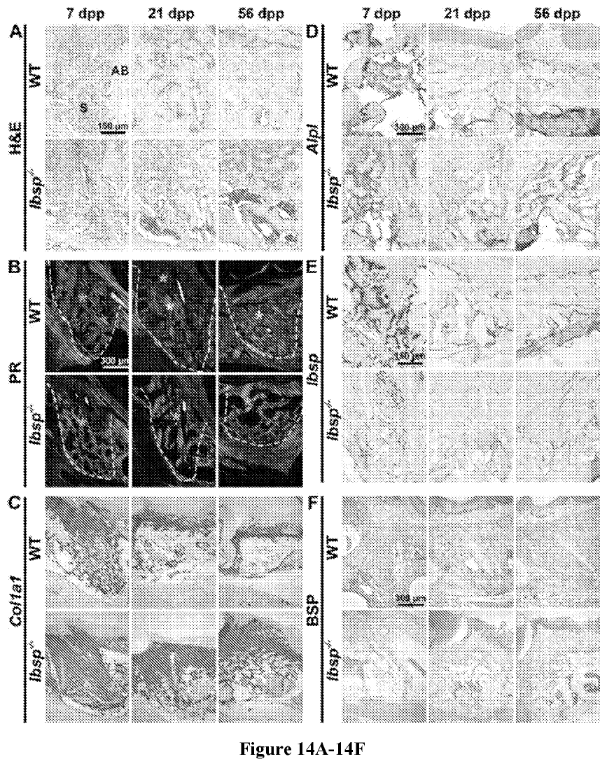

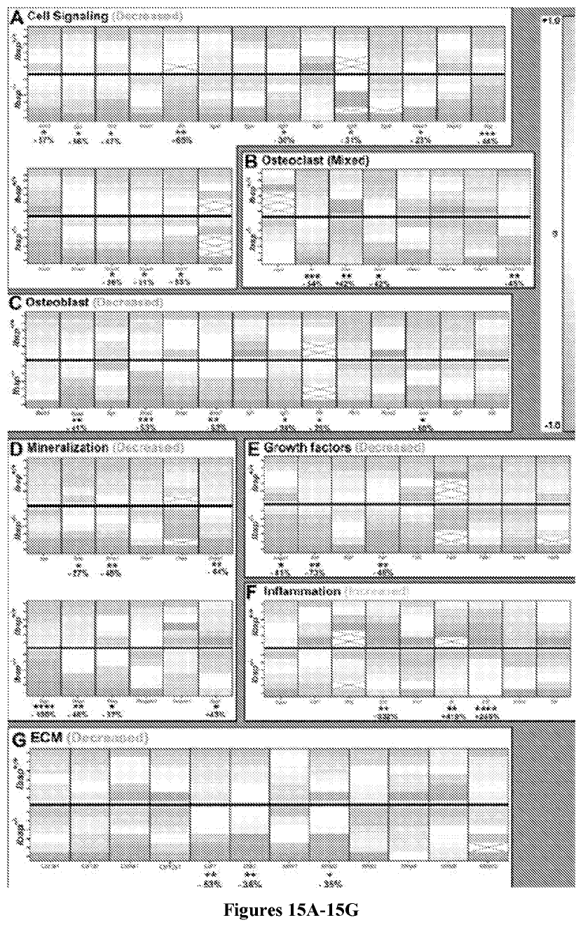

Many genes were dysregulated in Ibsp^{-/-} versus WT mice at 14 dpp. Of the 83 genes that amplified, 37 (45%) were significantly up- or downregulated. We evaluated genes by functional groups, organizing them into heatmaps to show scale of change and statistically significant differences (FIG. 15A-15G) the cell signaling group, 11 of 20 genes (55%) were reduced, including Axin2, Gli1, Gli2, Ihh, Itgb1, Itgb5, Mapk1, Nog, Smad3, Smad4, and Wnt11 (FIG. 15A). The osteoclast group showed mixed regulation, with 3 genes downregulated (Ar, Dkk1, and Tnfrsf11b) and 1 upregulated (Ctsk), out of 8 total (38%) (FIG. 15B). In the osteoblast group, 6 of 14 genes (43%) were reduced, including Bglap, Bmp2, Bmp7, Igflr, Pth, and Sost (FIG. 15C). Changes in the mineralization group genes were mixed, with 7 of 12 genes (58%) dysregulated, including increased Spp1 and decreased Ank, Bmp3, Enpp1, Ibsp, Mepe, and Phex (FIG. 15D). Growth factors were decreased, with 3 of 9 (33%) reduced, including Angpt1, Egf, and Fgf1 (FIG. 15E). Inflammation-associated genes were increased, with 3 of 9 (33%) genes elevated, including Il1b, Il6, and Il10 (FIG. 15F). Extracellular matrix genes were downregulated, with 3 of 12 genes (25%) reduced, including Ddr1, Ddr2, and Mmp2 (FIG. 15G). Table 1 includes the full list of comparisons for Ibsp^{-/-} versus WT mice at 14 dpp. Additional comparisons include WT mice at 14 versus 0 dpp (Table 2), Ibsp^{-/-} mice at 14 versus 0 dpp (Table 3), and Ibsp^{I} versus WT mice at 0 dpp (Table 4).

To further explore the unexpected inflammation-associated gene dysregulation, we performed IHC for neutrophils and macrophages (FIG. 9A-9B). Both types of immune cells were present in high numbers at 1 dpp. Neutrophils were absent from WT and Ibsp^{-/-} sockets at 7 to 56 dpp. Macrophages were present near bone surfaces in both WT and Ibsp^{-/-} mice at early ages. Macrophage numbers decreased over time in WT but remained elevated in the large persistent marrow spaces in Ibsp^{-/-} mice.

TABLE 1
Comparison of Gene Expression in Ibsp^{-/-} vs. WT Mice at 14 dpp
(healing). Gene symbol, name, probe identification number, relative quantification,
percent change (Ibsp^{-/-} vs. WT at 14 dpp) and p-value are provided.

| | | | | % | P- |
|---|---|---|---|---|---|
| | Name | Probe ID | RQ | Change | value |
| Ibsp | integrin binding sialoprotein | Mm00492555_m1 | 0.000586 | −99.9% | 0.00002 |
| Dspp | dentin sialophosphoprotein | Mm00515667_g1 | 0.060661 | −93.9% | 0.14629 |
| Pth | parathyroid hormone | Mm00451600_g1 | 0.131997 | −86.8% | 0.11643 |
| Wnt3a | wingless-type MMTV | Mm00437337_m1 | 0.133174 | −86.7% | 0.07771 |
| | integration site family, member | | | | |
| | 3A | | | | |
| Itgb6 | integrin beta 6 | Mm00445330_m1 | 0.134433 | −86.6% | 0.12001 |
| Acp5 | acid phosphatase 5, tartrate | Mm00475698_m1 | 5.633436 | 463.3% | 0.23970 |
| | resistant | | | | |
| Il6 | interleukin 6 | Mm00446190_m1 | 5.192057 | 419.2% | 0.00683 |
| Il1b | interleukin 1 beta | Mm00434228_m1 | 4.320136 | 332.0% | 0.00332 |
| Egf | epidermal growth factor | Mm00438696_m1 | 0.270837 | −72.9% | 0.00233 |
| Il10 | interleukin 10 | Mm01288386_m1 | 3.546064 | 254.6% | 0.00079 |
| Fgf3 | fibroblast growth factor 3 | Mm00433290_m1 | 0.288178 | −71.2% | 0.43116 |
| Gli1 | GLI-Kruppel family member | Mm00494654_m1 | 0.337675 | −66.2% | 0.01636 |
| | GLI1 | | | | |
| Ihh | Indian hedgehog | Mm00439613_m1 | 0.352126 | −64.8% | 0.00513 |
| Wnt11 | wingless-type MMTV | Mm00437327_g1 | 0.366072 | −63.4% | 0.03146 |
| | integration site family, member | | | | |
| | 11 | | | | |
| Mmp10 | matrix metallopeptidase 10 | Mm01168399_m1 | 0.399980 | −60.0% | 0.38443 |
| Sost | sclerostin | Mm00470479_m1 | 0.404033 | −59.6% | 0.01116 |
| Axin2 | axin2 | Mm00443610_m1 | 0.431153 | −56.9% | 0.01841 |
| Ar | androgen receptor | Mm00442688_m1 | 0.454701 | −54.5% | 0.00014 |
| Bmp7 | bone morphogenetic protein 7 | Mm00432102_m1 | 0.466036 | −53.4% | 0.00585 |
| Bmp2 | bone morphogenetic protein 2 | Mm01340178_m1 | 0.472073 | −52.8% | 0.00044 |
| Ddr1 | discoidin domain receptor | Mm01273496_m1 | 0.474077 | −52.6% | 0.00824 |
| | family, member 1 | | | | |
| Mepe | matrix extracellular | Mm02525159_s1 | 0.515723 | −48.4% | 0.00236 |
| | phosphoglycoprotein | | | | |
| Bmp3 | bone morphogenetic protein 3 | Mm00557790_m1 | 0.517431 | −48.3% | 0.00359 |
| Gli2 | GLI-Kruppel family member | Mm01293117_m1 | 0.534704 | −46.5% | 0.01210 |
| | GLI2 | | | | |
| Fgf1 | fibroblast growth factor 1 | Mm00438906_m1 | 0.538098 | −46.2% | 0.00109 |
| Tnfrsf11b | tumor necrosis factor receptor | Mm00435451_m1 | 0.546852 | −45.3% | 0.00898 |
| | superfamily, member 11b | | | | |
| Nog | noggin | Mm01297833_s1 | 0.556086 | −44.4% | 0.00071 |
| Enpp1 | ectonucleotide pyrophosphatase/ | Mm01193758_m1 | 0.562291 | −43.8% | 0.00241 |
| | phosphodiesterase 1 | | | | |
| Dkk1 | dickkopf homolog 1 (Xenopus | Mm00438422_m1 | 0.580826 | −41.9% | 0.02025 |
| | laevis) | | | | |
| Fgf2 | fibroblast growth factor 2 | Mm00433287_m1 | 0.581698 | −41.8% | 0.05422 |
| Angpt1 | angiopoietin 1 | Mm00456503_m1 | 0.588072 | −41.2% | 0.02457 |
| Bglap | bone gamma carboxyglutamate | Mm03413826_mH | 0.589196 | −41.1% | 0.00235 |
| | protein | | | | |
| Postn | periostin, osteoblast specific | Mm01284913_g1 | 0.600067 | −40.0% | 0.06019 |
| | factor | | | | |
| Mmp8 | matrix metallopeptidase 8 | Mm00439509_m1 | 1.663060 | 66.3% | 0.11492 |
| Tnf | tumor necrosis factor | Mm00443258_m1 | 1.657422 | 65.7% | 0.28250 |
| Phex | phosphate regulating | Mm00448119_m1 | 0.631487 | −36.9% | 0.04741 |
| | endopeptidase homolog | | | | |
| Ddr2 | discoidin domain receptor | Mm00445615_m1 | 0.635317 | −36.5% | 0.00233 |
| | family, member 2 | | | | |
| Igf1r | insulin-like growth factor 1 | Mm00802831_m1 | 0.635760 | −36.4% | 0.01876 |
| | receptor | | | | |
| Tgfb1 | transforming growth factor, beta | Mm01178820_m1 | 0.641389 | −35.9% | 0.22639 |
| | 1 | | | | |
| Smad3 | SMAD family member 3 | Mm01170760_m1 | 0.641989 | −35.8% | 0.03775 |
| Mmp2 | matrix metallopeptidase 2 | Mm00439498_m1 | 0.648001 | −35.2% | 0.04256 |
| Sp7 | Sp7 transcription factor 7 | Mm04933803_m1 | 0.655205 | −34.5% | 0.06210 |
| Ifng | interferon gamma | Mm01168134_m1 | 1.494251 | 49.4% | 0.63419 |
| Bmp4 | bone morphogenetic protein 4 | Mm00432087_m1 | 0.675544 | −32.4% | 0.06581 |
| Itgb5 | integrin beta 5 | Mm00439825_m1 | 0.686061 | −31.4% | 0.01299 |
| Smad4 | SMAD family member 4 | Mm03023996_m1 | 0.686592 | −31.3% | 0.03218 |
| Alpl | alkaline phosphatase | Mm00475834_m1 | 0.688025 | −31.2% | 0.08493 |
| Egfr | epidermal growth factor receptor | Mm01187858_m1 | 0.697091 | −30.3% | 0.07480 |
| Spp1 | secreted phosphoprotein 1 | Mm00436767_m1 | 1.433867 | 43.4% | 0.02845 |
| Itgb1 | integrin beta 1 (fibronectin | Mm01253230_m1 | 0.701729 | −29.8% | 0.03168 |
| | receptor beta) | | | | |
| Ctsk | cathepsin K | Mm00484039_m1 | 1.417178 | 41.7% | 0.00974 |
| Il10ra | interleukin 10 receptor, alpha | Mm00434147_m1 | 1.384913 | 38.5% | 0.12804 |
| Ank | progressive ankylosis | Mm00445040_m1 | 0.731534 | −26.8% | 0.01822 |
| Vegfb | vascular endothelial growth | Mm00442102_m1 | 0.750845 | −24.9% | 0.19052 |
| | factor B | | | | |
| Pth1r | parathyroid hormone 1 receptor | Mm00441046_m1 | 0.751168 | −24.9% | 0.03682 |
| Dmp1 | dentin matrix protein 1 | Mm01208363_m1 | 0.755476 | −24.5% | 0.10793 |
| Mapk1 | mitogen-activated protein kinase | Mm00442479_m1 | 0.770268 | −23.0% | 0.04270 |
| | 1 | | | | |
| Grem1 | gremlin 1 | Mm00488615_s1 | 0.771654 | −22.8% | 0.09149 |
| Mapk3 | mitogen-activated protein kinase 3 | Mm01278702_gH | 0.780417 | −22.0% | 0.17634 |
| Smad1 | SMAD family member 1 | Mm00484723_m1 | 0.780936 | −21.9% | 0.10553 |
| Vegfa | vascular endothelial growth | Mm00437306_m1 | 0.790283 | −21.0% | 0.17300 |
| | factor A | | | | |
| Cd44 | CD44 antigen | Mm01277165_m1 | 0.791886 | −20.8% | 0.28676 |
| Serpine1 | serpin peptidase inhibitor | Mm01204470_m1 | 0.828718 | −17.1% | 0.70493 |
| Mmp9 | matrix metallopeptidase 9 | Mm00442991_m1 | 1.245419 | 24.5% | 0.22763 |
| Igf1 | insulin-like growth factor 1 | Mm00439560_m1 | 0.834212 | −16.6% | 0.24226 |
| Itgav | integrin alpha V | Mm01339539_m1 | 0.844866 | −15.5% | 0.25045 |
| Il1r1 | interleukin 1 receptor, type I | Mm00434237_m1 | 1.180323 | 18.0% | 0.29451 |
| Itgb3 | integrin beta 3 | Mm00443980_m1 | 1.178553 | 17.9% | 0.36168 |
| Phospho1 | phosphatase, orphan 1 | Mm00462190_m1 | 0.852440 | −14.8% | 0.39146 |
| Bgn | biglycan | Mm01191753_m1 | 0.857808 | −14.2% | 0.40573 |
| Col1a2 | collagen, type I, alpha 2 | Mm00483888_m1 | 0.864139 | −13.6% | 0.23530 |
| Col4a1 | collagen, type IV, alpha 1 | Mm01210125_m1 | 0.881671 | −11.8% | 0.56191 |
| Bach1 | BTB and CNC homology 1 | Mm01344527_m1 | 0.884681 | −11.5% | 0.17307 |
| Mmp3 | matrix metallopeptidase 3 | Mm00440295_m1 | 1.101544 | 10.2% | 0.86202 |
| Runx2 | runt related transcription factor 2 | Mm00501584_m1 | 0.913402 | −8.7% | 0.79338 |
| Vdr | vitamin D receptor | Mm00437297_m1 | 0.914355 | −8.6% | 0.53881 |
| Icam1 | intercellular adhesion molecule 1 | Mm00516023_m1 | 1.089009 | 8.9% | 0.68343 |
| Col1a1 | collagen, type I, alpha 1 | Mm00801666_gl | 0.924389 | −7.6% | 0.77562 |
| Itga5 | integrin alpha 5 (fibronectin | Mm01305873_g1 | 0.937679 | −6.2% | 0.61892 |
| | receptor alpha) | | | | |
| Csf1 | colony stimulating factor 1 | Mm00432686_m1 | 0.942795 | −5.7% | 0.70463 |
| | (macrophage) | | | | |
| Tnfsf11 | tumor necrosis factor (ligand) | Mm00441906_m1 | 1.058653 | 5.9% | 0.72469 |
| | superfamily, member 11 | | | | |
| Tnfrsf1a | tumor necrosis factor receptor | Mm01182929_m1 | 1.045334 | 4.5% | 0.50644 |
| | superfamily, member 1a | | | | |
| Nfatc1 | nuclear factor of activated T cells | Mm01265944_m1 | 0.960590 | −3.9% | 0.80877 |
| Col12a1 | collagen, type XII, alpha 1 | Mm01148576_m1 | 1.009022 | 0.9% | 0.97534 |
| 18S | Eukaryotic 18S rRNA | Hs99999901_s1 | N/A | N/A | N/A |
| Gapdh | glyceraldehyde-3-phosphate | Mm99999915_g1 | N/A | N/A | N/A |
| | dehydrogenase | | | | |
| Gusb | glucuronidase, beta | Mm01197698_m1 | N/A | N/A | N/A |
| Ppib | peptidylprolyl isomerase B | Mm00478295_m1 | N/A | N/A | N/A |
| Ahsg | alpha-2-HS-glycoprotein | Mm01145470_m1 | N/A | N/A | N/A |
| Dlx5 | distal-less homeobox 5 | Mm00438430_m1 | N/A | N/A | N/A |
| Il13 | interleukin 13 | Mm00434204_m1 | N/A | N/A | N/A |
| Il17a | interleukin 17A | Mm00439618_m1 | N/A | N/A | N/A |
| Il4 | interleukin 4 | Mm00445259_m1 | N/A | N/A | N/A |
| Mmp1a | matrix metallopeptidase 1a | Mm00473485_m1 | N/A | N/A | N/A |
| Mmp7 | matrix metallopeptidase 7 | Mm00487724_m1 | N/A | N/A | N/A |
| Shh | sonic hedgehog | Mm00436528_m1 | N/A | N/A | N/A |

TABLE 2
Comparison of Gene Expression in WT Mice at 0 vs. 14 dpp (before and during
healing). Gene symbol, name, probe identification number, relative quantification,
percent change (Ibsp^{-/-} vs. WT at 14 dpp) and p-value are provided.

| | | | | % | P- |
|---|---|---|---|---|---|
| | Name | Probe ID | RQ | Change | value |
| Pth1r | parathyroid hormone 1 receptor | Mm00441046_m1 | 10.5041 | 950.41% | 0.07146 |
| Wnt3a | wingless-type MMTV | Mm00437337_m1 | 3.1128 | 211.28% | 0.30992 |
| | integration site family, member | | | | |
| | 3A | | | | |
| Acp5 | acid phosphatase 5, tartrate | Mm00475698_m1 | 0.3736 | −62.64% | 0.44596 |
| | resistant | | | | |
| Ifng | interferon gamma | Mm01168134_m1 | 0.4426 | −55.74% | 0.30131 |
| Mmp3 | matrix metallopeptidase 3 | Mm00440295_m1 | 0.4606 | −53.94% | 0.03871 |
| Mmp10 | matrix metallopeptidase 10 | Mm01168399_m1 | 2.1341 | 113.41% | 0.26045 |
| Dspp | dentin sialophosphoprotein | Mm00515667_g1 | 0.5096 | −49.04% | 0.54651 |
| Bglap | bone gamma carboxyglutamate | Mm03413826_mH | 1.9615 | 96.15% | 0.00064 |
| | protein | | | | |
| Il6 | interleukin 6 | Mm00446190_m1 | 1.8966 | 89.66% | 0.12162 |
| Ibsp | integrin binding sialoprotein | Mm00492555_m1 | 1.8919 | 89.19% | 0.01209 |
| Phex | phosphate regulating | Mm00448119_m1 | 1.8618 | 86.18% | 0.04231 |
| | endopeptidase homolog | | | | |
| Alpl | alkaline phosphatase, | Mm00475834_m1 | 1.8424 | 84.24% | 0.01434 |
| | liver/bone/kidney | | | | |
| Mmp8 | matrix metallopeptidase 8 | Mm00439509_m1 | 0.5545 | −44.55% | 0.21723 |
| Dkk1 | dickkopf homolog 1 (Xenopus | Mm00438422_m1 | 1.7303 | 73.03% | 0.01920 |
| | laevis) | | | | |
| Dmp1 | dentin matrix protein 1 | Mm01208363_m1 | 1.6644 | 66.44% | 0.03210 |
| Egf | epidermal growth factor | Mm00438696_m1 | 0.6243 | −37.57% | 0.28954 |
| Mmp9 | matrix metallopeptidase 9 | Mm00442991_m1 | 1.6007 | 60.07% | 0.04464 |
| Sp7 | Sp7 transcription factor 7 | Mm04933803_m1 | 1.5861 | 58.61% | 0.00695 |
| Ihh | Indian hedgehog | Mm00439613_m1 | 0.6432 | −35.68% | 0.26640 |
| Bmp3 | bone morphogenetic protein 3 | Mm00557790_m1 | 1.5408 | 54.08% | 0.02422 |
| Phospho1 | phosphatase, orphan 1 | Mm00462190_m1 | 1.4879 | 48.79% | 0.00908 |
| Vdr | vitamin D receptor | Mm00437297_m1 | 1.4818 | 48.18% | 0.07290 |
| Sost | sclerostin | Mm00470479_m1 | 1.4719 | 47.19% | 0.04103 |
| Bmp4 | bone morphogenetic protein 4 | Mm00432087_m1 | 1.4712 | 47.12% | 0.05002 |
| Bmp7 | bone morphogenetic protein 7 | Mm00432102_m1 | 1.4671 | 46.71% | 0.08222 |
| Itgb6 | integrin beta 6 | Mm00445330_m1 | 1.4537 | 45.37% | 0.32658 |
| Col1a2 | collagen, type I, alpha 2 | Mm00483888_m1 | 1.4433 | 44.33% | 0.04172 |
| Col1a1 | collagen, type I, alpha 1 | Mm00801666_g1 | 1.4412 | 44.12% | 0.08640 |
| Ctsk | cathepsin K | Mm00484039_m1 | 1.4130 | 41.30% | 0.04636 |
| Pth | parathyroid hormone | Mm00451600_g1 | 1.4091 | 40.91% | 0.05992 |
| Itgb5 | integrin beta 5 | Mm00439825_m1 | 1.3981 | 39.81% | 0.25093 |
| Itgav | integrin alpha V | Mm01339539_m1 | 1.3926 | 39.26% | 0.12547 |
| Il1b | interleukin 1 beta | Mm00434228_m1 | 1.3902 | 39.02% | 0.41249 |
| Col4a1 | collagen, type IV, alpha 1 | Mm01210125_m1 | 1.3775 | 37.75% | 0.15465 |
| Cd44 | CD44 antigen | Mm01277165_m1 | 1.3656 | 36.56% | 0.07226 |
| Grem1 | gremlin 1 | Mm00488615_s1 | 1.3622 | 36.22% | 0.31754 |
| Vegfa | vascular endothelial growth | Mm00437306_m1 | 1.3598 | 35.98% | 0.01073 |
| | factor A | | | | |
| Mepe | matrix extracellular | Mm02525159_s1 | 1.3478 | 34.78% | 0.08233 |
| | phosphoglycoprotein with | | | | |
| | ASARM motif | | | | |
| Smad3 | SMAD family member 3 | Mm01170760_m1 | 1.3324 | 33.24% | 0.10453 |
| Igf1 | insulin-like growth factor 1 | Mm00439560_m1 | 1.3153 | 31.53% | 0.13975 |
| Enpp1 | ectonucleotide pyrophosphatase | Mm01193758_m1 | 1.3148 | 31.48% | 0.13718 |
| Ddr2 | discoidin domain receptor | Mm00445615_m1 | 1.3093 | 30.93% | 0.11325 |
| | family, member 2 | | | | |
| Nog | noggin | Mm01297833_s1 | 1.3009 | 30.09% | 0.09329 |
| Gli1 | GLI-Kruppel family member | Mm00494654_m1 | 1.2841 | 28.41% | 0.44694 |
| | GLI1 | | | | |
| Itga5 | integrin alpha 5 | Mm01305873_g1 | 1.2566 | 25.66% | 0.16826 |
| Fgf3 | fibroblast growth factor 3 | Mm00433290_m1 | 1.2527 | 25.27% | 0.82672 |
| Vegfb | vascular endothelial growth | Mm00442102_m1 | 1.2501 | 25.01% | 0.23953 |
| | factor B | | | | |
| Runx2 | runt related transcription factor 2 | Mm00501584_m1 | 1.2482 | 24.82% | 0.49084 |
| Smad1 | SMAD family member 1 | Mm00484723_m1 | 1.2418 | 24.18% | 0.07810 |
| Bmp2 | bone morphogenetic protein 2 | Mm01340178_m1 | 1.2360 | 23.60% | 0.23047 |
| Csf1 | colony stimulating factor 1 | Mm00432686_m1 | 1.2330 | 23.30% | 0.23120 |
| | (macrophage) | | | | |
| Bgn | biglycan | Mm01191753_m1 | 1.2294 | 22.94% | 0.29372 |
| Tgfb1 | transforming growth factor, beta | Mm01178820_m1 | 1.2218 | 22.18% | 0.22391 |
| | 1 | | | | |
| Axin2 | axin2 | Mm00443610_m1 | 0.8189 | −18.11% | 0.50889 |
| Ar | androgen receptor | Mm00442688_m1 | 1.2205 | 22.05% | 0.09369 |
| Nfatc1 | nuclear factor of activated T cells | Mm01265944_m1 | 1.2128 | 21.28% | 0.24384 |
| Wnt11 | wingless-type MMTV | Mm00437327_g1 | 0.8364 | −16.36% | 0.60714 |
| | integration site family, member | | | | |
| | 11 | | | | |
| Smad4 | SMAD family member 4 | Mm03023996_m1 | 1.1919 | 19.19% | 0.07571 |
| Tnf | tumor necrosis factor | Mm00443258_m1 | 1.1732 | 17.32% | 0.58252 |
| Mmp2 | matrix metallopeptidase 2 | Mm00439498_m1 | 1.1629 | 16.29% | 0.48433 |
| Mapk1 | mitogen-activated protein kinase | Mm00442479_m1 | 1.1530 | 15.30% | 0.26006 |
| | 1 | | | | |
| Bach1 | BTB and CNC homology 1 | Mm01344527_m1 | 1.1344 | 13.44% | 0.29887 |
| Tnfrsf11b | tumor necrosis factor receptor | Mm00435451_m1 | 1.1307 | 13.07% | 0.66370 |
| | superfamily, member 11b | | | | |
| Itgb3 | integrin beta 3 | Mm00443980_m1 | 1.1281 | 12.81% | 0.64474 |
| Fgf1 | fibroblast growth factor 1 | Mm00438906_m1 | 1.1266 | 12.66% | 0.49799 |
| Il1r1 | interleukin 1 receptor, type I | Mm00434237_m1 | 1.1260 | 12.60% | 0.39107 |
| Col12a1 | collagen, type XII, alpha 1 | Mm01148576_m1 | 1.1216 | 12.16% | 0.68112 |
| Itgb1 | integrin beta 1 | Mm01253230_m1 | 1.1182 | 11.82% | 0.39998 |
| Angpt1 | angiopoietin 1 | Mm00456503_m1 | 1.1181 | 11.81% | 0.67770 |
| Postn | periostin, osteoblast specific | Mm01284913_g1 | 0.8997 | −10.03% | 0.67250 |
| | factor | | | | |
| Gli2 | GLI-Kruppel family member | Mm01293117_m1 | 1.1111 | 11.11% | 0.70131 |
| | GLI2 | | | | |
| Mapk3 | mitogen-activated protein kinase | Mm01278702_gH | 1.1071 | 10.71% | 0.53411 |
| | 3 | | | | |
| Serpine1 | serpin peptidase inhibitor | Mm01204470_m1 | 1.0905 | 9.05% | 0.89103 |
| Icam1 | intercellular adhesion molecule 1 | Mm00516023_m1 | 0.9292 | −7.08% | 0.67649 |
| Fgf2 | fibroblast growth factor 2 | Mm00433287_m1 | 0.9342 | −6.58% | 0.81655 |
| Ddr1 | discoidin domain receptor | Mm01273496_m1 | 1.0690 | 6.90% | 0.77747 |
| | family, member 1 | | | | |
| Il10ra | interleukin 10 receptor, alpha | Mm00434147_m1 | 0.9450 | −5.50% | 0.73478 |
| Il10 | interleukin 10 | Mm01288386_m1 | 0.9585 | −4.15% | 0.88884 |
| Egfr | epidermal growth factor receptor | Mm01187858_m1 | 0.9614 | −3.86% | 0.74851 |
| Tnfsf11 | tumor necrosis factor (ligand) | Mm00441906_m1 | 0.9782 | −2.18% | 0.91129 |
| | superfamily, member 11 | | | | |
| Ank | progressive ankylosis | Mm00445040_m1 | 1.0217 | 2.17% | 0.87202 |
| Tnfrsf1a | tumor necrosis factor receptor | Mm01182929_m1 | 0.9813 | −1.87% | 0.82176 |
| | superfamily, member 1a | | | | |
| Spp1 | secreted phosphoprotein 1 | Mm00436767_m1 | 0.9816 | −1.84% | 0.91307 |
| Igf1r | insulin-like growth factor 1 | Mm00802831_m1 | 0.9975 | −0.25% | 0.98682 |
| | receptor | | | | |
| 18S | Eukaryotic 18S rRNA | Hs99999901_s1 | N/A | N/A | N/A |
| Gapdh | glyceraldehyde-3-phosphate | Mm99999915_g1 | N/A | N/A | N/A |
| | dehydrogenase | | | | |
| Gusb | glucuronidase, beta | Mm01197698_m1 | N/A | N/A | N/A |
| Ppib | peptidylprolyl isomerase B | Mm00478295_m1 | N/A | N/A | N/A |
| Ahsg | alpha-2-HS-glycoprotein | Mm01145470_m1 | N/A | N/A | N/A |
| Dlx5 | distal-less homeobox 5 | Mm00438430_m1 | N/A | N/A | N/A |
| Il13 | interleukin 13 | Mm00434204_m1 | N/A | N/A | N/A |
| Il17a | interleukin 17A | Mm00439618_m1 | N/A | N/A | N/A |
| Il4 | interleukin 4 | Mm00445259_m1 | N/A | N/A | N/A |
| Mmp1a | matrix metallopeptidase 1a | Mm00473485_m1 | N/A | N/A | N/A |
| Mmp7 | matrix metallopeptidase 7 | Mm00487724_m1 | N/A | N/A | N/A |
| Shh | sonic hedgehog | Mm00436528_m1 | N/A | N/A | N/A |

TABLE 3
Comparison of Gene Expression in Ibsp^{-/-} Mice at 0 vs. 14 dpp (before
and during healing). Gene symbol, name, probe identification number, relative quantification,
percent change (Ibsp^{-/-} vs. WT at 14 dpp) and p-value are provided.

| | | | | % | P- |
|---|---|---|---|---|---|
| Gene | Name | Probe ID | RQ | Change | value |
| Il6 | interleukin 6 | Mm00446190_m1 | 12.2364 | 1123.64% | 0.0005 |
| Pth1r | parathyroid hormone 1 receptor | Mm00441046_m1 | 0.1233 | −87.67% | 0.2243 |
| Itgb6 | integrin beta 6 | Mm00445330_m1 | 0.1736 | −82.64% | 0.1617 |
| Mmp10 | matrix metallopeptidase 10 | Mm01168399_m1 | 0.1754 | −82.46% | 0.1238 |
| Il1b | interleukin 1 beta | Mm00434228_m1 | 5.1282 | 412.82% | 0.0186 |
| Egf | epidermal growth factor | Mm00438696_m1 | 0.2004 | −79.96% | 0.0023 |
| Wnt11 | wingless-type MMTV integration site | Mm00437327_g1 | 0.2461 | −75.39% | 0.0089 |
| | family, member 11 | | | | |
| Ibsp | integrin binding sialoprotein | Mm00492555_m1 | 4.0016 | 300.16% | 0.0589 |
| Dspp | dentin sialophosphoprotein | Mm00515667_g1 | 0.3416 | −65.84% | 0.5775 |
| Postn | periostin | Mm01284913_g1 | 0.3851 | −61.49% | 0.0006 |
| Il10 | interleukin 10 | Mm01288386_m1 | 2.5386 | 153.86% | 0.0230 |
| Serpine1 | serpin peptidase inhibitor | Mm01204470_m1 | 0.4392 | −56.08% | 0.1235 |
| Tnfrsf11b | tumor necrosis factor receptor | Mm00435451_m1 | 0.4410 | −55.90% | 0.0169 |
| | superfamily, member 11b | | | | |
| Acp5 | acid phosphatase 5, tartrate resistant | Mm00475698_m1 | 2.1629 | 116.29% | 0.0025 |
| Bmp2 | bone morphogenetic protein 2 | Mm01340178_m1 | 0.4949 | −50.51% | 0.0001 |
| Wnt3a | wingless-type MMTV integration site | Mm00437337_m1 | 0.4953 | −50.47% | 0.2883 |
| | family, member 3A | | | | |
| Axin2 | axin2 | Mm00443610_m1 | 0.4961 | −50.39% | 0.0125 |
| Ar | androgen receptor | Mm00442688_m1 | 0.5205 | −47.95% | 0.0085 |
| Mmp9 | matrix metallopeptidase 9 | Mm00442991_m1 | 1.8799 | 87.99% | 0.0016 |
| Tnf | tumor necrosis factor | Mm00443258_m1 | 1.8760 | 87.60% | 0.1649 |
| Ifng | interferon gamma | Mm01168134_m1 | 1.7456 | 74.56% | 0.3561 |
| Itgb1 | integrin beta 1 (fibronectin receptor | Mm01253230_m1 | 0.5758 | −42.42% | 0.0025 |
| | beta) | | | | |
| Ctsk | cathepsin K | Mm00484039_m1 | 1.6809 | 68.09% | 0.0080 |
| Vdr | vitamin D receptor | Mm00437297_m1 | 1.6717 | 67.17% | 0.0188 |
| Ihh | Indian hedgehog | Mm00439613_m1 | 0.6027 | −39.73% | 0.1256 |
| Enpp1 | ectonucleotide | Mm01193758_m1 | 0.6080 | −39.20% | 0.0086 |
| | pyrophosphatase/phosphodiesterase 1 | | | | |
| Il10ra | interleukin 10 receptor, alpha | Mm00434147_m1 | 1.6302 | 63.02% | 0.0681 |
| Col4a1 | collagen, type IV, alpha 1 | Mm01210125_m1 | 1.6274 | 62.74% | 0.0344 |
| Fgf1 | fibroblast growth factor 1 | Mm00438906_m1 | 0.6201 | −37.99% | 0.0341 |
| Phospho1 | phosphatase, orphan 1 | Mm00462190_m1 | 1.6012 | 60.12% | 0.0475 |
| Shh | sonic hedgehog | Mm00436528_m1 | 0.6280 | −37.20% | 0.8639 |
| Mmp2 | matrix metallopeptidase 2 | Mm00439498_m1 | 0.6563 | −34.37% | 0.0331 |
| Bmp7 | bone morphogenetic protein 7 | Mm00432102_m1 | 0.6593 | −34.07% | 0.0766 |
| Fgf2 | fibroblast growth factor 2 | Mm00433287_m1 | 0.6684 | −33.16% | 0.0101 |
| Col12a1 | collagen, type XII, alpha 1 | Mm01148576_m1 | 0.6755 | −32.45% | 0.1638 |
| Ank | progressive ankylosis | Mm00445040_m1 | 0.6789 | −32.11% | 0.0288 |
| Nfatc1 | nuclear factor of activated T cells | Mm01265944_m1 | 1.4542 | 45.42% | 0.0471 |
| Igf1r | insulin-like growth factor 1 receptor | Mm00802831_m1 | 0.6917 | −30.83% | 0.0837 |
| Mmp8 | matrix metallopeptidase 8 | Mm00439509_m1 | 0.6964 | −30.36% | 0.1430 |
| Il1r1 | interleukin 1 receptor, type I | Mm00434237_m1 | 1.4313 | 43.13% | 0.1993 |
| Itgb3 | integrin beta 3 | Mm00443980_m1 | 1.4300 | 43.00% | 0.0215 |
| Angpt1 | angiopoietin 1 | Mm00456503_m1 | 0.7010 | −29.90% | 0.0984 |
| Phex | phosphate regulating endopeptidase | Mm00448119_m1 | 1.3769 | 37.69% | 0.0574 |
| | homolog | | | | |
| Ddr1 | discoidin domain receptor family, | Mm01273496_m1 | 0.7513 | −24.87% | 0.2235 |
| | member 1 | | | | |
| Col1a1 | collagen, type I, alpha 1 | Mm00801666_g1 | 1.3300 | 33.00% | 0.3373 |
| Icam1 | intercellular adhesion molecule 1 | Mm00516023_m1 | 1.3255 | 32.55% | 0.3046 |
| Ddr2 | discoidin domain receptor family, | Mm00445615_m1 | 0.7643 | −23.57% | 0.1152 |
| | member 2 | | | | |
| Tnfsf11 | tumor necrosis factor (ligand) | Mm00441906_m1 | 1.3077 | 30.77% | 0.1484 |
| | superfamily, member 11 | | | | |
| Csf1 | colony stimulating factor 1 | Mm00432686_m1 | 1.3075 | 30.75% | 0.0377 |
| | (macrophage) | | | | |
| Bmp3 | bone morphogenetic protein 3 | Mm00557790_m1 | 1.2983 | 29.83% | 0.6065 |
| Pth | parathyroid hormone | Mm00451600_g1 | 1.2735 | 27.35% | 0.1209 |
| Fgf3 | fibroblast growth factor 3 | Mm00433290_m1 | 1.2024 | 20.24% | 0.4666 |
| Bglap | bone gamma carboxyglutamate protein | Mm03413826_mH | 1.2023 | 20.23% | 0.3407 |
| Mmp3 | matrix metallopeptidase 3 | Mm00440295_m1 | 1.1905 | 19.05% | 0.8103 |
| Col1a2 | collagen, type I, alpha 2 | Mm00483888_m1 | 1.1823 | 18.23% | 0.1761 |
| Itga5 | integrin alpha 5 (fibronectin receptor | Mm01305873_g1 | 1.1740 | 17.40% | 0.2439 |
| | alpha) | | | | |
| Smad3 | SMAD family member 3 | Mm01170760_m1 | 1.1740 | 17.40% | 0.4791 |
| Mapk3 | mitogen-activated protein kinase 3 | Mm01278702_gH | 0.8570 | −14.30% | 0.5241 |
| Alpl | alkaline phosphatase, liver/bone/kidney | Mm00475834_m1 | 1.1548 | 15.48% | 0.4947 |
| Itgav | integrin alpha V | Mm01339539_m1 | 1.1509 | 15.09% | 0.3268 |
| Smad4 | SMAD family member 4 | Mm03023996_m1 | 0.8699 | −13.01% | 0.4563 |
| Sp7 | Sp7 transcription factor 7 | Mm04933803_m1 | 1.1425 | 14.25% | 0.6208 |
| Nog | noggin | Mm01297833_s1 | 1.1416 | 14.16% | 0.5876 |
| Tgfb1 | transforming growth factor, beta 1 | Mm01178820_m1 | 0.8837 | −11.63% | 0.7197 |
| Bach1 | BTB and CNC homology 1 | Mm01344527_m1 | 1.1059 | 10.59% | 0.2419 |
| Gli1 | GLI-Kruppel family member GLI1 | Mm00494654_m1 | 0.9060 | −9.40% | 0.7758 |
| Gli2 | GLI-Kruppel family member GLI2 | Mm01293117_m1 | 1.0926 | 9.26% | 0.7643 |
| Egfr | epidermal growth factor receptor | Mm01187858_m1 | 0.9245 | −7.55% | 0.7016 |
| Spp1 | secreted phosphoprotein 1 | Mm00436767_m1 | 1.0787 | 7.87% | 0.5927 |
| Dmp1 | dentin matrix protein 1 | Mm01208363_m1 | 1.0785 | 7.85% | 0.7452 |
| Bgn | biglycan | Mm01191753_m1 | 0.9350 | −6.50% | 0.7961 |
| Bmp4 | bone morphogenetic protein 4 | Mm00432087_m1 | 0.9352 | −6.48% | 0.6977 |
| Sost | sclerostin | Mm00470479_m1 | 1.0688 | 6.88% | 0.8340 |
| Cd44 | CD44 antigen | Mm01277165_m1 | 1.0546 | 5.46% | 0.8242 |
| Igf1 | insulin-like growth factor 1 | Mm00439560_m1 | 1.0495 | 4.95% | 0.8217 |
| Greml | gremlin 1 | Mm00488615_s1 | 0.9555 | −4.45% | 0.8172 |
| Runx2 | runt related transcription factor 2 | Mm00501584_m1 | 1.0443 | 4.43% | 0.8976 |
| Tnfrsf1a | tumor necrosis factor receptor | Mm01182929_m1 | 1.0338 | 3.38% | 0.5468 |
| | superfamily, member 1a | | | | |
| Mepe | matrix extracellular | Mm02525159_s1 | 0.9722 | −2.78% | 0.8872 |
| | phosphoglycoprotein with ASARM | | | | |
| | motif | | | | |
| Mapk1 | mitogen-activated protein kinase 1 | Mm00442479_m1 | 0.9749 | −2.51% | 0.8104 |
| Vegfa | vascular endothelial growth factor A | Mm00437306_m1 | 0.9830 | −1.70% | 0.9472 |
| Smad1 | SMAD family member 1 | Mm00484723_m1 | 0.9833 | −1.67% | 0.9070 |
| Dkk1 | dickkopf homolog 1 (Xenopus laevis) | Mm00438422_m1 | 1.0131 | 1.31% | 0.9491 |
| Vegfb | vascular endothelial growth factor B | Mm00442102_m1 | 0.9951 | −0.49% | 0.9833 |
| 18S | Eukaryotic 18S rRNA | Hs99999901_s1 | N/A | N/A | N/A |
| Gapdh | glyceraldehyde-3-phosphate | Mm99999915_g1 | N/A | N/A | N/A |
| | dehydrogenase | | | | |
| Gusb | glucuronidase, beta | Mm01197698_m1 | N/A | N/A | N/A |
| Ppib | peptidylprolyl isomerase B | Mm00478295_m1 | N/A | N/A | N/A |
| Ahsg | alpha-2-HS-glycoprotein | Mm01145470_m1 | N/A | N/A | N/A |
| Dlx5 | distal-less homeobox 5 | Mm00438430_m1 | N/A | N/A | N/A |
| Il13 | interleukin 13 | Mm00434204_m1 | N/A | N/A | N/A |
| Il17a | interleukin 17A | Mm00439618_m1 | N/A | N/A | N/A |
| Il4 | interleukin 4 | Mm00445259_m1 | N/A | N/A | N/A |
| Mmp1a | matrix metallopeptidase 1a | Mm00473485_m1 | N/A | N/A | N/A |
| Mmp7 | matrix metallopeptidase 7 | Mm00487724_m1 | N/A | N/A | N/A |
| Shh | sonic hedgehog | Mm00436528_m1 | N/A | N/A | N/A |

TABLE 4
Comparison of Gene Expression in Ibsp^{-/-} vs. WT Mice at 0 dpp (baseline).
Gene symbol, name, probe identification number, relative quantification, percent
change (Ibsp^{-/-} vs. WT at 14 dpp) and p-value are provided.

| | | | | % | P- |
|---|---|---|---|---|---|
| Gene | Name | Probe ID | RQ | Change | value |
| Ibsp | integrin binding sialoprotein | Mm00492555_m1 | 0.0003 | −99.97% | 0.0000 |
| Dspp | dentin sialophosphoprotein | Mm00515667_g1 | 0.0741 | −92.59% | 0.1045 |
| Ifng | interferon gamma | Mm01168134_m1 | 0.2800 | −72.00% | 0.0148 |
| Ihh | Indian hedgehog | Mm00439613_m1 | 0.2883 | −71.17% | 0.0140 |
| Gli1 | GLI-Kruppel family member GLI1 | Mm00494654_m1 | 0.3763 | −62.37% | 0.0109 |
| Mmp3 | matrix metallopeptidase 3 | Mm00440295_m1 | 0.3841 | −61.59% | 0.1629 |
| Sost | sclerostin | Mm00470479_m1 | 0.4066 | −59.34% | 0.0151 |
| Pth1r | parathyroid hormone 1 receptor | Mm00441046_m1 | 2.4558 | 145.58% | 0.5448 |
| Dlx5 | distal-less homeobox 5 | Mm00438430_m1 | 0.4522 | −54.78% | 0.2117 |
| Smad3 | SMAD family member 3 | Mm01170760_m1 | 0.5312 | −46.88% | 0.0210 |
| Gli2 | GLI-Kruppel family member GLI2 | Mm01293117_m1 | 0.5435 | −45.65% | 0.0733 |
| Mepe | matrix extracellular | Mm02525159_s1 | 0.5765 | −42.35% | 0.0284 |
| | phosphoglycoprotein | | | | |
| Axin2 | axin2 | Mm00443610_m1 | 0.6010 | −39.90% | 0.0885 |
| Vdr | vitamin D receptor | Mm00437297_m1 | 0.6112 | −38.88% | 0.0117 |
| Sp7 | Sp7 transcription factor 7 | Mm04933803_m1 | 0.6190 | −38.10% | 0.0385 |
| Icam1 | intercellular adhesion molecule 1 | Mm00516023_m1 | 0.6258 | −37.42% | 0.0195 |
| Phospho1 | phosphatase, orphan 1 | Mm00462190_m1 | 0.6309 | −36.91% | 0.0131 |
| Pth | parathyroid hormone | Mm00451600_g1 | 0.6381 | −36.19% | 0.0246 |
| Tnfsf11 | tumor necrosis factor (ligand) | Mm00441906_m1 | 0.6385 | −36.15% | 0.0375 |
| | superfamily, member 11 | | | | |
| Itgb3 | integrin beta 3 | Mm00443980_m1 | 0.6411 | −35.89% | 0.0047 |
| Phex | phosphate regulating endopeptidase | Mm00448119_m1 | 0.6414 | −35.86% | 0.0280 |
| | homolog | | | | |
| Angpt1 | angiopoietin 1 | Mm00456503_m1 | 0.6428 | −35.72% | 0.0326 |
| Il10ra | interleukin 10 receptor, alpha | Mm00434147_m1 | 0.6653 | −33.47% | 0.0664 |
| Bglap | bone gamma carboxyglutamate | Mm03413826_mH | 0.6744 | −32.56% | 0.0902 |
| | protein | | | | |
| Fgf3 | fibroblast growth factor 3 | Mm00433290_m1 | 0.6785 | −32.15% | 0.6159 |
| Nfatc1 | nuclear factor of activated T cells | Mm01265944_m1 | 0.6854 | −31.46% | 0.0091 |
| Runx2 | runt related transcription factor 2 | Mm00501584_m1 | 0.6859 | −31.41% | 0.2020 |
| Egfr | epidermal growth factor receptor | Mm01187858_m1 | 0.6864 | −31.36% | 0.0716 |
| Nog | noggin | Mm01297833_s1 | 0.6949 | −30.51% | 0.2248 |
| Ddr1 | discoidin domain receptor family, | Mm01273496_m1 | 0.7127 | −28.73% | 0.1948 |
| | member 1 | | | | |
| Cd44 | CD44 antigen | Mm01277165_m1 | 0.7234 | −27.66% | 0.0821 |
| Dkk1 | dickkopf homolog 1 (Xenopus | Mm00438422_m1 | 0.7237 | −27.63% | 0.2157 |
| | laevis) | | | | |
| Bmp3 | bone morphogenetic protein 3 | Mm00557790_m1 | 0.7415 | −25.85% | 0.1355 |
| Tgfb1 | transforming growth factor, beta 1 | Mm01178820_m1 | 0.7445 | −25.55% | 0.1201 |
| Mmp8 | matrix metallopeptidase 8 | Mm00439509_m1 | 0.7461 | −25.39% | 0.3303 |
| Fgf2 | fibroblast growth factor 2 | Mm00433287_m1 | 0.7537 | −24.63% | 0.1825 |
| Mmp10 | matrix metallopeptidase 10 | Mm01168399_m1 | 1.3214 | 32.14% | 0.7287 |
| Smad4 | SMAD family member 4 | Mm03023996_m1 | 0.7586 | −24.14% | 0.0988 |
| Dmp1 | dentin matrix protein 1 | Mm01208363_m1 | 0.7654 | −23.46% | 0.3130 |
| Il1r1 | interleukin 1 receptor, type I | Mm00434237_m1 | 0.7704 | −22.96% | 0.3602 |
| Ddr2 | discoidin domain receptor family, | Mm00445615_m1 | 0.7736 | −22.64% | 0.1048 |
| | member 2 | | | | |
| Il10 | interleukin 10 | Mm01288386_m1 | 1.2857 | 28.57% | 0.4049 |
| Alpl | alkaline phosphatase, | Mm00475834_m1 | 0.7789 | −22.11% | 0.3096 |
| | liver/bone/kidney | | | | |
| Fgf1 | fibroblast growth factor 1 | Mm00438906_m1 | 0.7836 | −21.64% | 0.2433 |
| Grem1 | gremlin 1 | Mm00488615_s1 | 0.7895 | −21.05% | 0.3079 |
| Col12a1 | collagen, type XII, alpha 1 | Mm01148576_m1 | 1.2524 | 25.24% | 0.3927 |
| Mapk3 | mitogen-activated protein kinase 3 | Mm01278702_gH | 0.8031 | −19.69% | 0.2270 |
| Vegfa | vascular endothelial growth factor A | Mm00437306_m1 | 0.8080 | −19.20% | 0.3251 |
| Bach1 | BTB and CNC homology 1 | Mm01344527_m1 | 0.8084 | −19.16% | 0.0284 |
| Col1a1 | collagen, type I, alpha 1 | Mm00801666_g1 | 0.8108 | −18.92% | 0.4163 |
| Mapk1 | mitogen-activated protein kinase 1 | Mm00442479_m1 | 0.8179 | −18.21% | 0.1116 |
| Itgb5 | integrin beta 5 | Mm00439825_m1 | 0.8181 | −18.19% | 0.5362 |
| Ctsk | cathepsin K | Mm00484039_m1 | 1.2145 | 21.45% | 0.3115 |
| Acp5 | acid phosphatase 5, tartrate resistant | Mm00475698_m1 | 0.8243 | −17.57% | 0.3897 |
| Col4a1 | collagen, type IV, alpha 1 | Mm01210125_m1 | 0.8283 | −17.17% | 0.3596 |
| Wnt3a | wingless-type MMTV integration | Mm00437337_m1 | 0.8319 | −16.81% | 0.8217 |
| | site family, member 3A | | | | |
| Igf1r | insulin-like growth factor 1 receptor | Mm00802831_m1 | 0.8326 | −16.74% | 0.3738 |
| Igf1 | insulin-like growth factor 1 | Mm00439560_m1 | 0.8356 | −16.44% | 0.3491 |
| Ar | androgen receptor | Mm00442688_m1 | 0.8365 | −16.35% | 0.2637 |
| Il1b | interleukin 1 beta | Mm00434228_m1 | 1.1937 | 19.37% | 0.7773 |
| Itgav | integrin alpha V | Mm01339539_m1 | 0.8405 | −15.95% | 0.3341 |
| Itga5 | integrin alpha 5 (fibronectin receptor | Mm01305873_g1 | 0.8414 | −15.86% | 0.1128 |
| | alpha) | | | | |
| Tnf | tumor necrosis factor | Mm00443258_m1 | 0.8414 | −15.86% | 0.5897 |
| Spp1 | secreted phosphoprotein 1 | Mm00436767_m1 | 1.1691 | 16.91% | 0.3650 |
| Postn | periostin | Mm01284913_g1 | 1.1456 | 14.56% | 0.6079 |
| Itgb1 | integrin beta 1 (fibronectin receptor | Mm01253230_m1 | 1.1437 | 14.37% | 0.2803 |
| | beta) | | | | |
| Bgn | biglycan | Mm01191753_m1 | 0.8811 | −11.89% | 0.6430 |
| Vegfb | vascular endothelial growth factor B | Mm00442102_m1 | 0.8896 | −11.04% | 0.6704 |
| Mmp2 | matrix metallopeptidase 2 | Mm00439498_m1 | 0.8897 | −11.03% | 0.5976 |
| Smad1 | SMAD family member 1 | Mm00484723_m1 | 0.8976 | −10.24% | 0.4129 |
| Tnfrsf11b | tumor necrosis factor receptor | Mm00435451_m1 | 0.9007 | −9.93% | 0.7657 |
| | superfamily, member 11b | | | | |
| Wnt11 | wingless-type MMTV integration | Mm00437327_g1 | 1.1087 | 10.87% | 0.7875 |
| | site family, member 11 | | | | |
| Csf1 | colony stimulating factor 1 | Mm00432686_m1 | 0.9047 | −9.53% | 0.4014 |
| | (macrophage) | | | | |
| Col1a2 | collagen, type I, alpha 2 | Mm00483888_m1 | 0.9065 | −9.35% | 0.5209 |
| Egf | epidermal growth factor | Mm00438696_m1 | 1.0781 | 7.81% | 0.8825 |
| Tnfrsf1a | tumor necrosis factor receptor | Mm01182929_m1 | 0.9308 | −6.92% | 0.2184 |
| | superfamily, member 1a | | | | |
| Serpine1 | serpin peptidase inhibitor | Mm01204470_m1 | 1.0734 | 7.34% | 0.8959 |
| Bmp4 | bone morphogenetic protein 4 | Mm00432087_m1 | 0.9456 | −5.44% | 0.7516 |
| Ank | progressive ankylosis | Mm00445040_m1 | 0.9465 | −5.35% | 0.7281 |
| Enpp1 | ectonucleotide | Mm01193758_m1 | 0.9502 | −4.98% | 0.7680 |
| | pyrophosphatase/phosphodiesterase | | | | |
| | 1 | | | | |
| Bmp7 | bone morphogenetic protein 7 | Mm00432102_m1 | 0.9538 | −4.62% | 0.8440 |
| Itgb6 | integrin beta 6 | Mm00445330_m1 | 0.9678 | −3.22% | 0.9353 |
| Il6 | interleukin 6 | Mm00446190_m1 | 0.9750 | −2.50% | 0.9332 |
| Mmp9 | matrix metallopeptidase 9 | Mm00442991_m1 | 1.0134 | 1.34% | 0.9380 |
| 18S | Eukaryotic 18S rRNA | Hs99999901_s1 | N/A | N/A | N/A |
| Gapdh | glyceraldehyde-3-phosphate | Mm99999915_g1 | N/A | N/A | N/A |
| | dehydrogenase | | | | |
| Gusb | glucuronidase, beta | Mm01197698_m1 | N/A | N/A | N/A |
| Ppib | peptidylprolyl isomerase B | Mm00478295_m1 | N/A | N/A | N/A |
| Ahsg | alpha-2-HS-glycoprotein | Mm01145470_m1 | N/A | N/A | N/A |
| Dlx5 | distal-less homeobox 5 | Mm00438430_m1 | N/A | N/A | N/A |
| Il13 | interleukin 13 | Mm00434204_m1 | N/A | N/A | N/A |
| Il17a | interleukin 17A | Mm00439618_m1 | N/A | N/A | N/A |
| Il4 | interleukin 4 | Mm00445259_m1 | N/A | N/A | N/A |
| Mmp1a | matrix metallopeptidase 1a | Mm00473485_m1 | N/A | N/A | N/A |
| Mmp7 | matrix metallopeptidase 7 | Mm00487724_m1 | N/A | N/A | N/A |
| Shh | sonic hedgehog | Mm00436528_m1 | N/A | N/A | N/A |

### Discussion

We used a mouse model to study the functional importance of BSP in alveolar bone healing after tooth extraction. Ibsp mRNA and BSP protein were strongly expressed during healing. Compared to WT, Ibsp^{-/-} mice exhibited reduced bone volume and density, persistence of woven bone with large marrow spaces, and elevated Col1a1 and Alpl mRNA expression. Defective alveolar bone healing in Ibsp^{-/-} mice was associated with substantial dysregulation of genes associated with cell signaling, growth factors, osteoblasts, osteoclasts, mineralization, extracellular matrix, and inflammation. We conclude that BSP is a critical and nonredundant factor for alveolar bone repair, and its absence disrupts multiple major pathways involved in appropriate healing.

Defective Alveolar Bone Healing in Mice Lacking Bone Sialoprotein. Extraction of molars is a challenge model used to study alveolar bone healing in several animals, including mice, rats, and pigs (Pang et al. 2015; Vieira et al. 2015; Avivi-Arber et al. 2018; Yuan et al. 2018; Arioka et al. 2019; Pan et al. 2020). This approach interrogates the biology of intramembranous ossification, as opposed to endochondral ossification that occurs in fracture or surgical defect models in long bones. Insights are relevant to periodontal health, oral manifestations of skeletal diseases, and dental implant-based therapies. Bone healing is a complex, multistep process with early stages including granulation tissue, inflammatory cell infiltration, cell proliferation, angiogenesis, and fibrous tissue formation; intermediate stages including osteoblast differentiation and woven bone formation; and later stages marked by bone remodeling and maturation (Vieira et al. 2015).

Our adapted mouse model of molar socket healing was consistent with previous reports of fibrous tissue and woven bone at 7 dpp, and the majority of the socket filled with remodeling bone by 21 dpp and remodeled compact bone by 56 dpp. Critical outcomes for successful bone healing are formation of sufficient bone quantity and quality. In the absence of BSP, alveolar bone healing was defective in both volume and density. Histological analysis of Ibsp^{-/-} mice showed accumulation of woven bone and large marrow spaces that persisted in con-junction with elevation of early healing markers, Col1a1 and Alpl. These observations suggest that in the absence of BSP, alveolar bone healing could not progress to more mature bone, as underscored by qPCR results summarized below.

A challenging aspect of analyzing this model is that the original borders of the socket become increasingly difficult to identify as healing proceeds. We developed an innovative approach to increase reproducibility and detect smaller, more localized differences by microCT. Registering all maxillae to a standard model ensured optimal orientation, which was further refined by use of an average socket ROI object map overlaid onto healing maxillae where the original locations were no longer evident because of healing. After comparing multiple methods for measuring healing that had substantial shortcomings, this registration/overlay approach proved reproducible and was the most automated, reducing bias and uncertainty of manual tracing. Notably, this approach also allows bone healing to be evaluated irrespective of baseline differences that may exist in gene-edited mouse models.

Insights into BSP Functions in Alveolar Bone Healing. We performed a qPCR gene array to identify molecular mechanisms associated with defective bone healing in the absence of BSP. Our approach targeted 92 genes associated with functions relevant to bone healing: signaling, growth factors, inflammation, osteoblast and osteoclast biology, extracellular matrix, and mineralization. On a global level, PCA showed unsurprisingly that WT and Ibsp^{-/-} bone have substantial baseline differences at 0 dpp. More surprisingly, WT gene expression in healing versus baseline did not much vary, while healing in Ibsp^{-/-} mice prompted a dramatic shift in gene expression indicating substantial dysregulation and providing insights into the defective healing process. Based on implicated functions of BSP from in vitro and in vivo studies, we anticipated perturbations to primarily affect genes related to osteoblasts, osteoclasts, and mineralization. Surprisingly, all functional groups were substantially dysregulated, with 45% of tested genes showing significant up- or downregulation in Ibsp^{-/-} versus WT mice.

Major molecular signaling pathways were downregulated in healing bone in Ibsp^{-/-} versus WT mice. These included Ihh, Gli1, and Gli2 of the Hedgehog (Hh) pathway, important in skeletal development, bone repair, and socket healing (Hojo et al. 2012; Pang et al. 2015; Yang et al. 2015; Shi et al. 2017; Ohba 2020). The TGF-β/BMP/Smad pathway, critical for bone formation and repair, was also implicated by decreased Smad3/4 expression (Zou et al. 2021). Itgb1 and Itgb5, integrins associated with RGD signaling, were decreased in Ibsp^{-/-} versus WT healing bone. Disruptions in these and other integrins affect bone formation and resorption (Docheva et al. 2014). The Wnt signaling pathway was substantially affected, with changes in Nog, Wnt11, Sost, Dkk1, Axin2, Pth, and Igflr. Wnt signaling is important in alveolar bone repair (Yuan et al. 2018).

Several growth factors were decreased in Ibsp^{-/-} versus WT healing bone, all of which have roles in angiogenesis. Angpt1 regulates blood vessel formation, Egf stimulates cell differentiation and is expressed during alveolar bone healing, and Fgf1 has broad cellular induction properties and is also expressed during alveolar bone repair (Vieira et al. 2015).

Primary osteoblasts from Ibsp^{-/-} mice show decreased differentiation markers, including Sp7 and Bglap (Malaval et al. 2008). Although many of the genes and pathways discussed above affect osteoblast differentiation and function, additional osteoblast-selective genes were downregulated in the absence of BSP, including Bmp2 and Bmp7 from the TGF-β/BMP/Smad pathway, Pth, Igflr, and Bglap, with downward trends in Runx2 and Sp7 (Chan et al. 2021; Zou et al. 2021).

Osteoclast-associated genes were altered during healing in Ibsp^{-/-} mice. Primary osteoclasts from Ibsp^{-/-} mice show decreased differentiation and function (Malaval et al. 2008; Boudiffa et al. 2010). Increased osteoclast activity would result from decreased Tnfrsf11b (osteoprotegerin) and increased Ctsk (cathepsin K). Ar (androgen receptor) was decreased in Ibsp^{-/-} versus WT mice. Knockout of Ar in mice increased osteoclast activity (Kawano et al. 2003). TRAP staining suggested trends toward increased numbers of osteoclast-like cells in Ibsp^{-/-} versus WT mice at all time points, potentially contributing to lack of bone maturation.

Most mineralization-associated genes tested were down-regulated in Ibsp^{-/-} versus WT mice. Ank and Enpp1, which increase pyrophosphate levels to control mineralization, were downregulated and previously found to regulate periodontal mineralization in parallel to BSP (Ao et al. 2017; Nagasaki et al. 2021). Bmp3, a negative regulator of BMP signaling, was reduced (Daluiski et al. 2001). Increased Spp1 and reduced Mepe and Phex could contribute to inhibition of mineralization (Foster et al. 2018; Zhang et al. 2020).

Inflammation plays an important role in bone healing, particularly in early stages (Vieira et al. 2015). Surprisingly, pro-inflammatory genes Il6 and Il1b were increased more than 300% in Ibsp^{-/-} versus WT bone, although BSP has not been directly linked with inflammation pathways. In contrast, anti-inflammatory cytokine Il10 was significantly upregulated in Ibsp^{-/-} mice. RNA isolated for qPCR specifically excluded gingiva; therefore, expression profiles primarily reflect healing bone. Histological examination throughout healing did not identify substantial differences in immune cells between WT and Ibsp^{-/-} mice, although Ibsp^{-/-} alveolar bone featured persistent and enlarged marrow spaces that included macrophages. Altered inflammation-associated factors may also result from other cells present in healing; for example, fibroblasts, osteoblasts, and/or osteocytes could be producing inflammation-associated genes. Because associations between BSP and immune response are previously unreported, experiments are ongoing to better understand this unexpected observation. A more detailed discussion of altered gene expression and affected pathways is included below.

Lack of BSP Dysregulates Gene Expression during Healing. We performed a qPCR gene array to identify molecular mechanisms associated with defective bone healing in the absence of BSP. Our approach targeted 92 genes associated with functions relevant to bone healing: signaling, growth factors, inflammation, osteoblast and osteoclast biology, extracellular matrix, and mineralization.

On a global level, principal component analysis (PCA) of qPCR results showed unsurprisingly that WT and Ibsp^{-/-} bone have substantial baseline differences, i.e. differences that already exist at the initial time point of 0 dpp. WT gene expression in healing vs. baseline was very similar, with almost complete overlap between these experiment groups in the PCA plot (FIG. 8A-8B). In contrast, healing in Ibsp^{-/-} mice prompted a dramatic shift in gene expression as shown by distinct grouping of 14 vs. 0 dpp in the PCA plot. This indicates substantial dysregulation in alveolar bone healing in the absence of BSP.

Based on implicated functions of BSP from in vitro and in vivo studies, we anticipated perturbations to primarily affect genes in the functional groups, Osteoblasts, Osteoclasts, and Mineralization. Surprisingly, all functional groups analyzed were substantially dysregulated, with 45% of tested genes showing significant up- or down-regulation in Ibsp^{-/-} vs. WT mice. These changes are discussed in more detail below.

Our qPCR approach found numerous dysregulated functional groups during Ibsp^{-/-} vs. WT alveolar bone healing. The most succinct summary of the global expression changes is that, in the absence of BSP, there is low osteogenic potential, substantial dysregulation of mineralization-associated genes, and increased inflammation. These gene expression changes provide key insights into the defective alveolar bone healing in Ibsp^{-/-} mice documented by microCT and histology. Changes in all the functional groups are described in more detail below and can be reviewed in FIGS. 15A-15G and Table 1.

BSP Ablation Disrupts Several Key Signaling Pathways. In the Cell Signaling functional group, 11 of 20 genes were significantly down-regulated in the absence of BSP. One of the pathways most highly down-regulated in Ibsp^{-/-} vs. WT alveolar bone during healing, was the Hedgehog (Hh) signaling pathway. Indian hedgehog (Ihh) was decreased 65%, while Hh activators Gli1 and Gli2 were decreased by 66% and 47%, respectively. The Hh pathway contributes to cell functions including proliferation, migration, angiogenesis, stem cell regeneration, and bone morphogenetic protein (BMP) signaling, and Hh signaling is important in both endochondral and intramembranous ossification, bone repair, and socket healing (Haycraft and Serra 2008; Hojo et al. 2012; Ohba 2020; Pang et al. 2015; Serra 2008; Shi et al. 2017; Yang et al. 2015). Genetic ablation of Ihh causes ossification defects in lone bones and palate (Levi et al. 2011; Maeda et al. 2007; Razzaque et al. 2005). Deletion of Gli1 and Gli2 individually or in combination impairs osteoblast differentiation and bone formation (Hojo et al. 2012; Kitaura et al. 2014), with constitutive activation of Gli1 also disturbing osteogenesis (Joeng and Long 2013). Reduction of all these Hh-related factors during healing in Ibsp^{-/-} mice could contribute to defective alveolar bone healing.

Another major bone-associated cell signaling pathway affected by absence of BSP was the transforming growth factor β (TGF-β)/BMP/Smad pathway, long understood to have major importance in bone and periodontal biology (Rakian et al. 2013; Ripamonti et al. 2009; Ripamonti and Petit 2009; Ripamonti et al. 2006; Rosen 2006; Wu et al. 2016; Zou et al. 2021). Smads are key intermediate signaling molecules in TGF-β/BMP signaling, and Smad3 and Smad4 mRNA levels were decreased more than 30% in Ibsp^{-/-} vs. WT alveolar bone during healing. Both Smad3 and 4 are important for several key cell functions, including migration and differentiation. Genetic disruption of Smad3 and 4 have been shown in several studies to obstruct proper bone formation and homeostasis (Balooch et al. 2005; Moon et al. 2016; Salazar et al. 2013a; Salazar et al. 2013b; Tan et al. 2007). BMP inhibitor, noggin (Nog), which regulates skeletal development (Wijgerde et al. 2005; Wu et al. 2003) was also down-regulated in Ibsp^{-/-} vs. WT healing alveolar bone.

The Wnt signaling pathway has major effects on multiple systems, including skeletal development and regeneration; these functions also affect the craniofacial region (Houschyar et al. 2018; Leucht et al. 2008; Minear et al. 2010; Regard et al. 2012; Schupbach et al. 2020). Wnt11, decreased over 60% in Ibsp^{-/-} vs. WT healing alveolar bone, has been linked by several studies to osteoblast differentiation (Boyan et al. 2018; Caetano da Silva et al. 2022; Friedman et al. 2009). AXIN2, a negative regulator of Wnt signaling (Katoh 2008), also serves a surrogate for Wnt signaling activity (due its role in a negative feedback loop), and Axin2 expression was reduced nearly 60% in Ibsp^{-/-} vs. WT bone, perhaps an indicator of depressed Wnt signaling. Wnt signaling was previously demonstrated to be important in alveolar bone socket healing in mice (Liu et al. 2019; Yuan et al. 2018).

Also disrupted in Ibsp^{-/-} mice were multiple integrins associated with arginine-glycine-aspartic acid (RGD) outside-in signaling, including genes Itgb1 (encoding integrin subunit β1) and Itgb5 (encoding integrin subunit (5), both decreased by about 30%, and a trend towards decreased expression of Itgb6 (encoding integrin subunit β6). These integrins are important mediators in osteoblast and osteoclast signaling and functions (Asai et al. 2014; Docheva et al. 2014; Lane et al. 2005; Olivares-Navarrete et al. 2017; Yang et al. 2020; Zhang et al. 2020b).

Overall, cell signaling during alveolar bone healing appeared to be substantially depressed by the absence of BSP, based on the genes interrogated by qPCR. This possibly relates to defective osteoblast differentiation, but also a myriad of other possible cell signaling effects that would be relevant to bone healing. Markers of osteoblasts are considered in the section below.

Decreased Growth Factor Expression with BSP Ablation. In the Growth factors functional group, 3 of 9 genes were significantly down-regulated in healing bone in the absence of BSP. These decreased factors have functions in angiogenesis and vascular development. Angiopoietin 1 (Angpt1) regulates blood vessel formation, epidermal growth factor (Egf) stimulates cell differentiation and is expressed during alveolar bone healing, and fibroblast growth factor 1 (Fgf1) has broad cellular induction properties and is also expressed during alveolar bone repair (Carmeliet and Jain 2011; Chim et al. 2013; Vieira et al. 2015). Other growth factors implicated in bone formation, e.g. Vegfa and Vegfb (Dai and Rabie 2007), were not differentially expressed between genotypes.

Decreased Osteoblast Markers in Healing Alveolar Bone in the Absence of BSP. In the Osteoblast functional group, 6 of 14 genes were significantly down-regulated in the absence of BSP. Several of these genes are osteoblast differentiation inducers, markers, and/or elements of signaling pathways summarized in the previous signaling section. BMP2 and BMP7 are both osteogenic members of the BMP signaling pathway, described above. Bmp2 and Bmp7 genes both have critical roles in skeletal development, including in the craniofacial region (Bandyopadhyay et al. 2006; Bonilla-Claudio et al. 2012; Rosen 2006). BMP2 and BMP7, individually or in tandem, show great potential to promote craniofacial regeneration (Koh et al. 2008; Zhao et al. 2005). Expression levels of both Bmp2 and Bmp7 were significantly decreased in Ibsp^{-/-} mice, with Bmp4 also trending towards lower expression. Expression levels of Runx2 and Sp7 (Osterix), master regulators of osteoblast differentiation, were not significantly affected by the absence of BSP. However, osteocalcin (Bglap), considered a marker for mature osteoblasts and regulator of bone mineralization (Karsenty 2017; Komori 2020), was decreased by 41% in Ibsp^{-/-} vs. WT bone. Expression of sclerostin (Sost), a Wnt signaling regulator and osteocyte marker (Dallas et al. 2013; Delgado-Calle et al. 2017), was decreased 60%. Primary osteoblasts from Ibsp^{-/-} mice show decreased differentiation markers, including Sp7 and Bglap, suggesting defects in osteoblastogenesis (Malaval et al. 2008). Decreased expression of osteoblast and osteocyte markers in this study may reflect the failure of healing bone in Ibsp^{-/-} mice to properly remodel and progress to mature, compact bone.

Mixed Regulation of Osteoclast-associated Genes in the Absence of BSP. In the Osteoclast functional group, 3 of 8 genes were down-regulated and one gene was increased in the absence of BSP. Osteoclast function, as regulated through receptor activator of NF-κB ligand (RANKL), RANK, osteoprotegerin (OPG), and other factors, and is critical for skeletal development, remodeling, and healing (Boyce and Xing 2008; Dougall et al. 1999; Kong et al. 1999; Manrique et al. 2015; Udagawa et al. 2000; Vieira et al. 2015). Tnfsf11, encoding key osteoclast differentiation inducer, RANKL, was not altered by absence of BSP. Tnfrsf11a, encoding the RANK receptor for RANKL was also not different between genotypes. Likewise, Nfatc1 a master transcription factor directing osteoclastogenesis (Ikeda and Takeshita 2016; Kurotaki et al. 2020), and Acp5, encoding key osteoclast marker, tartrate-resistant acid phosphatase (TRAP) (Hollberg et al. 2002), both remained unchanged. Other genes showed mixed regulation that might promote osteoclast function. Tnfrsf11b, encoding OPG, an inhibitor of osteoclastogenesis, was reduced by 45%. This would suggest an increased RANKL/OPG ratio in local tissues that would promote osteoclastogenesis. Ctsk, encoding osteoclast marker cathepsin K (Saftig et al. 2000), was increased 42%. Intriguingly, androgen receptor (Ar) expression was decreased in Ibsp^{-/-} vs. WT mice; a study on Ar knockout mice found increased osteoclast numbers in the absence of the receptor (Kawano et al. 2003). Previous in vitro and in vivo studies on Ibsp^{-/-} mice demonstrate reduced osteoclastogenesis potential and reduced osteoclast numbers and function in the absence of BSP (Boudiffa et al. 2010; Malaval et al. 2008; Wade-Gueye et al. 2012). Conversely, BSP in addition to RANKL promoted increased osteoclastogenesis (Valverde et al. 2005). We have documented increased numbers of osteoclasts associated with alveolar bone destruction in Ibsp^{-/-} mice, a discrepancy with long bone findings that has not yet been explained (Foster et al. 2015; Foster et al. 2013). TRAP staining suggested trends towards increased numbers of osteoclast-like cells in Ibsp^{-/-} vs. WT mice at all time points (though no significant differences were identified), potentially contributing to lack of bone maturation. However, the mixed regulation of osteoclast-associated genes remains unclear, as does the functionality of the osteoclasts present in healing alveolar bone in Ibsp^{-/-} mice.

Decreased Expression of Extracellular Matrix Factors in the Absence of BSP. In the Extracellular matrix (ECM) functional group, 3 of 12 genes were down-regulated in the absence of BSP. Genes encoding discoidin domain receptors 1 and 2 (Ddr1, Ddr2), non-integrin collagen receptors that act as receptor tyrosine kinases, were decreased 53 and 36%, respectively, in Ibsp^{-/-} vs. WT healing alveolar bone. Both DDRs are associated with skeletal development (Chou et al. 2020; Ge et al. 2016; Vogel et al. 2001; Zhang et al. 2015), and ablation of both is associated with periodontal breakdown (Chavez et al. 2019; Mohamed et al. 2020). The extracellular domains of DDR1 and 2 also regulate collagen fibrillogenesis and mineralization (Blissett et al. 2009; Flynn et al. 2010; Mihai et al. 2006; Tonniges et al. 2016). Matrix metalloproteinase 2 (Mmp2) was decreased 35% in the absence of BSP. MMP2 has critical functions in skeletal and craniofacial development, osteoblast and osteoclast differentiation, and ECM remodeling (Inoue et al. 2006; Mosig et al. 2007). Interestingly, BSP has been suggested to specifically bind and activate MMP2 (Fedarko et al. 2004). Effects of BSP absence on other ECM-associated were not significant, suggesting this group of factors was less affected than other functional groups.

Absence of BSP Alters Mineralization-Associated Gene Expression during Bone Healing. In the Mineralization functional group, 7 of 12 genes were down-regulated in the absence of BSP, marking this as one of the most affected functional groups. Unsurprisingly, Ibsp expression was not detected in Ibsp^{-/-} alveolar bone, confirming gene knockout. Interestingly, several gene expression modulations observed would be expected to favor of mineralization in Ibsp^{-/-} mice. Genes encoding the progressive ankylosis protein (Ank) and ectonucleotide pyrophosphatase phosphodiesterase 1 (Enpp1) were down-regulated 27% and 44%, respectively. Reduction expression of these genes decreases levels of inorganic pyrophosphate (PPᵢ), a mineralization inhibitor (Addison et al. 2007; Harmey et al. 2004; Ho et al. 2000; Rutsch et al. 2003). Knockout of Ank and Enpp1, individually or collectively, increased cementum and alveolar bone repair (Nagasaki et al. 2021; Rodrigues et al. 2011). Expression of Bmp3, encoding a secreted inhibitor of BMP signaling (Daluiski et al. 2001), was reduced nearly 50%. Matrix extracellular phosphoglycoprotein (Mepe) expression was reduced nearly 50% in the absence of BSP. MEPE is closely related to BSP and its expression by osteoblasts and osteocytes suggests its involvement in osteoblast differentiation and bone mineralization, which was confirmed by increased bone formation in mice lacking Mepe (Fisher and Fedarko 2003; Gowen et al. 2003). Unlike the reduced expression in healing bone documented here, Mepe is reported to be over-expressed in long bones of Ibsp^{-/-} vs. WT mice (Bouleftour et al. 2014).

Other gene expression changes in Ibsp^{-/-} vs. WT mice would be expected to negatively impact mineralization. Expression of Spp1, encoding osteopontin (OPN), was increased 43%. OPN is a closely related to BSP and proposed to be a mineralization inhibitor (Boskey et al. 2002; Fisher and Fedarko 2003; Harmey et al. 2004; Harmey et al. 2006; Hunter et al. 1996; Pampena et al. 2004). Genetic deletion of Spp1 in mice results in increased alveolar bone (Foster et al. 2018). Spp1 is over-expressed in in long bones of Ibsp^{-/-} vs. WT mice (Bouleftour et al. 2014). Absence of BSP reduced expression of the enzyme, Phex (Phosphate regulating endopeptidase homolog, X-linked) by 37%. Inactivating mutations in PHEX in humans cause X-linked hypophosphatemia (XLH; OMIM #307800), featuring hypophosphatemia and severely disturbed mineralization (Buss et al. 2020; Eicher et al. 1976; Foster et al. 2014). PHEX is highly expressed by odontoblasts and osteocytes, with low level expression by cementocytes (Ruchon et al. 2000; Zhang et al. 2020a). PHEX cleaves and inactivates acidic serine- and aspartate-rich motif (ASARM) peptides derived from ECM proteins, including OPN and MEPE, that can act as mineral inhibitors; loss-of-function in XLH results in abnormal localization of OPN in bones and teeth related to hypomineralization defects (Addison et al. 2009; Barros et al. 2013; Boukpessi et al. 2017; Salmon et al. 2014). The overall effects of BSP ablation on mineralization during alveolar bone healing, as measured by this set of genes, remains unclear because of the complex changes and interrelated and counter-regulatory functions of several of the factors. However, the number of genes affected and their high fold-change suggest dramatic dysregulation of mineralization.

Deletion of BSP Increases Expression of Inflammatory Genes during Bone Healing. Inflammation plays an important role in bone healing, particularly in early stages (Maruyama et al. 2020; Vieira et al. 2015). In the Inflammation functional group, 3 of 9 genes were highly increased in the absence of BSP. Specifically, two pro-inflammatory genes, interleukin 6 (Il6) and interleukin 1β (Il1b), were increased by 332% and 419%, respectively. Increased expression of IL-6, IL-1B, and other pro-inflammatory cytokines is linked to delayed alveolar bone healing (Colavite et al. 2019). However, an anti-inflammatory cytokine, interleukin 10 (Il10), was also significantly upregulated in Ibsp^{-/-} mice compared to controls during healing. IL-10 is associated with anti-inflammatory effects, i.e. pro-resolution of inflammation, and polarization of M1 to M2 macrophages that contribute to bone repair; however, IL-10 also affects osteoblast differentiation and skeletal development (Maruyama et al. 2020). Other inflammatory genes trended but did not show significant differences between genotypes. Notably, the RNA isolated for analysis specifically excluded the gingiva, so expression patterns are not derived from epithelial inflammation, but inflammation directly surrounding bone during healing. Histological examination throughout healing did not identify substantial differences in immune cells between WT and Ibsp^{-/-} mice, though Ibsp^{-/-} alveolar bone featured persistent and enlarged marrow spaces that contained numerous macrophages. Altered inflammation-associated factors may also result from other cells present in healing, e.g. fibroblasts, osteoblasts, and/or osteocytes could be producing inflammation-associated genes. Because associations between BSP and immune response are previously unreported, experiments are ongoing to better understand this unexpected observation.

### Conclusion

Nearly half of the genes analyzed by qPCR were dysregulated in Ibsp^{-/-} vs. WT alveolar bone during healing. This was a surprising finding. To date, hypothesized functions of BSP have been largely limited to osteoblast and osteoclast differentiation and regulation of mineralization. It is not possible to determine which changes are direct or indirect effects of BSP ablation, and whether they are directly associated with any of the conserved functional domains in BSP. To address these questions, we are currently preparing mouse models conditionally ablated for Ibsp in different cell populations, and gene edited mice and cells where functional domains of Ibsp have been inactivated to explore their specific functions. Studies in these models will provide further insights into the functions of BSP.

In summary, we found defective quantity and quality of alveolar bone healing in mice lacking BSP, with persistent woven bone with large marrow spaces and dysregulation of many genes associated with bone healing. One limitation is the inability to precisely define how gene changes are associated with ablation of BSP; there are likely direct and indirect effects at play. To address these, we are currently preparing mouse models conditionally ablated for Ibsp in different cell populations and gene-edited mice and cells where functional domains of Ibsp have been inactivated to explore their specific functions. Studies in these models will provide further insights into the functions of BSP.

### REFERENCES

Ao M, Chavez M B, Chu E Y, Hemstreet K C, Yin Y, Yadav M C, Millan J L, Fisher L W, Goldberg H A, Somerman M J, et al. 2017. Overlapping functions of bone sialoprotein and pyrophosphate regulators in directing cementogenesis. Bone. 105:134-147.
Arioka M, Zhang X, Li Z, Tulu U S, Liu Y, Wang L, Yuan X, Helms J A. 2019. Osteoporotic changes in the periodontium impair alveolar bone healing. J Dent Res. 98(4):450-458.
Avivi-Arber L, Avivi D, Perez M, Arber N, Shapira S. 2018. Impaired bone healing at tooth extraction sites in CD24-deficient mice: a pilot study. PLoS One. 13(2):e0191665.
Boudiffa M, Wade-Gueye N M, Guignandon A, Vanden-Bossche A, Sabido O, Aubin J E, Jurdic P, Vico L, Lafage-Proust M H, Malaval L. 2010. Bone sialoprotein deficiency impairs osteoclastogenesis and mineral resorption in vitro. J Bone Miner Res. 25(12):2669-2679.
Chan W C W, Tan Z, To M K T, Chan D. 2021. Regulation and role of transcription factors in osteogenesis. Int J Mol Sci. 22(11):5445.
Chavez M B, Chu E Y, Kram V, de Castro L F, Somerman M J, Foster B L. 2021. Guidelines for micro-computed tomography analysis of rodent dentoalveolar tissues. JBMR Plus. 5(3):e10474.
Daluiski A, Engstrand T, Bahamonde M E, Gamer L W, Agius E, Stevenson S L, Cox K, Rosen V, Lyons K M. 2001. Bone morphogenetic protein-3 is a negative regulator of bone density. Nat Genet. 27(1):84-88.
Docheva D, Popov C, Alberton P, Aszodi A. 2014. Integrin signaling in skeletal development and function. Birth Defects Res C Embryo Today. 102(1):13-36.
Foster B L, Ao M, Salmon C R, Chavez M B, Kolli T N, Tran A B, Chu E Y, Kantovitz K R, Yadav M, Narisawa S, et al. 2018. Osteopontin regulates dentin and alveolar bone development and mineralization. Bone. 107:196-207.
Foster B L, Ao M, Willoughby C, Soenjaya Y, Holm E, Lukashova L, Tran A B, Wimer H F, Zerfas P M, Nociti F H Jr. et al. 2015. Mineralization defects in cementum and craniofacial bone from loss of bone sialoprotein. Bone. 78:150-164.
Foster B L, Soenjaya Y, Nociti F H Jr, Holm E, Zerfas P M, Wimer H F, Holdsworth D W, Aubin J E, Hunter G K, Goldberg H A, et al. 2013. Deficiency in acellular cementum and periodontal attachment in BSP null mice. J Dent Res. 92(2):166-172.
Ganss B, Kim R H, Sodek J. 1999. Bone sialoprotein. Crit Rev Oral Biol Med. 10(1):79.
Goldberg H A, Hunter G K. 2012. Functional domains of bone sialoprotein. In: Goldberg M, editor. Phosphorylated extracellular matrix proteins of bone and dentin. Oak Park (IL): Bentham Science Publishers. p. 266-282.
Goldberg H A, Warner K J, Stillman M J, Hunter G K. 1996. Determination of the hydroxyapatite-nucleating region of bone sialoprotein. Connect Tissue Res. 35(1-4):385-392.
Gordon J A, Hunter G K, Goldberg H A. 2009. Activation of the mitogen-activated protein kinase pathway by bone sialoprotein regulates osteoblast differentiation. Cells Tissues Organs. 189(1-4):138-143.
Gordon J A, Tye C E, Sampaio A V, Underhill T M, Hunter G K, Goldberg H A. 2007. Bone sialoprotein expression enhances osteoblast differentiation and matrix mineralization in vitro. Bone. 41(3):462-473.
Hojo H, Ohba S, Yano F, Saito T, Ikeda T, Nakajima K, Komiyama Y, Nakagata N, Suzuki K, Takato T, et al. 2012. Gli1 protein participates in Hedgehog-mediated specification of osteoblast lineage during endochondral ossification. J Biol Chem. 287(21):17860-17869.
Holm E, Aubin J E, Hunter G K, Beier F, Goldberg H A. 2015. Loss of bone sialoprotein leads to impaired endochondral bone development and mineralization. Bone. 71:145-154.
Hunter G K, Goldberg H A. 1994. Modulation of crystal formation by bone phosphoproteins: role of glutamic acid-rich sequences in the nucleation of hydroxyapatite by bone sialoprotein. Biochem J. 302 (Pt 1):175-179.
Kawano H, Sato T, Yamada T, Matsumoto T, Sekine K, Watanabe T, Nakamura T, Fukuda T, Yoshimura K, Yoshizawa T, et al. 2003. Suppressive function of androgen receptor in bone resorption. Proc Natl Acad Sci USA. 100(16):9416-9421.
Malaval L, Monfoulet L, Fabre T, Pothuaud L, Bareille R, Miraux S, Thiaudiere E, Raffard G, Franconi J M, Lafage-Proust M H, et al. 2009. Absence of bone sialoprotein (BSP) impairs cortical defect repair in mouse long bone. Bone. 45(5):853-861.
Malaval L, Wade-Gueye N M, Boudiffa M, Fei J, Zirngibl R, Chen F, Laroche N, Roux J P, Burt-Pichat B, Duboeuf F, et al. 2008. Bone sialoprotein plays a functional role in bone formation and osteoclastogenesis. J Exp Med. 205(5):1145-1153.
Miwa H E, Gerken T A, Jamison O, Tabak L A. 2010. Isoform-specific O-glycosylation of osteopontin and bone sialoprotein by polypeptide N-acetylgalactosaminyltransferase-1. J Biol Chem. 285(2):1208-1219.
Nagasaki A, Nagasaki K, Chu E Y, Kear B D, Tadesse W D, Ferebee S E, Li L, Foster B L, Somerman M J. 2021. Ablation of pyrophosphate regulators promotes periodontal regeneration. J Dent Res. 100(6):639-647.
Ohba S. 2020. Hedgehog signaling in skeletal development: roles of Indian hedgehog and the mode of its action. Int J Mol Sci. 21(18):6665.
Pan J, Pilawski I, Yuan X, Arioka M, Ticha P, Tian Y, Helms J A. 2020. Interspecies comparison of alveolar bone biology: tooth extraction socket healing in mini pigs and mice. J Periodontol. 91(12):1653-1663.
Pang P, Shimo T, Takada H, Matsumoto K, Yoshioka N, Ibaragi S, Sasaki A. 2015. Expression pattern of sonic hedgehog signaling and calcitonin gene-related peptide in the socket healing process after tooth extraction. Biochem Biophys Res Commun. 467(1):21-26.
Pei X, Wang L, Chen C, Yuan X, Wan Q, Helms J A. 2017. Contribution of the PDL to osteotomy repair and implant osseointegration. J Dent Res. 96(8):909-916.
Shi Y, He G, Lee W C, Mckenzie J A, Silva M J, Long F. 2017. Gli1 identifies osteogenic progenitors for bone formation and fracture repair. Nat Commun. 8(1):2043.
Soenjaya Y, Foster B L, Nociti F H Jr, Ao M, Holdsworth D W, Hunter G K, Somerman M J, Goldberg H A. 2015. Mechanical forces exacerbate periodontal defects in BSP-null mice. J Dent Res. 94(9):1276-1285.
Tye C E, Rattray K R, Warner K J, Gordon J A, Sodek J, Hunter G K, Goldberg H A. 2003. Delineation of the hydroxyapatite-nucleating domains of bone sialoprotein. J Biol Chem. 278(10):7949-7955.
Vieira A E, Repeke C E, Ferreira Junior Sde B, Colavite P M, Biguetti C C, Oliveira R C, Assis G F, Taga R, Trombone A P, Garlet G P. 2015. Intramembranous bone healing process subsequent to tooth extraction in mice: micro-computed tomography, histomorphometric and molecular characterization. PLoS One. 10(5):e0128021.
Wade-Gueye N M, Boudiffa M, Vanden-Bossche A, Laroche N, Aubin J E, Vico L, Lafage-Proust M H, Malaval L. 2012. Absence of bone sialoprotein (BSP) impairs primary bone formation and resorption: the marrow ablation model under PTH challenge. Bone. 50(5):1064-1073.
Xu L, Zhang Z, Sun X, Wang J, Xu W, Shi L, Lu J, Tang J, Liu J, Su X. 2017. Glycosylation status of bone sialoprotein and its role in mineralization. Exp Cell Res. 360(2):413-420.
Yang J, Andre P, Ye L, Yang Y Z. 2015. The Hedgehog signaling pathway in bone formation. Int J Oral Sci. 7(2):73-79.
Yuan X, Pei X, Zhao Y, Tulu U S, Liu B, Helms J A. 2018. A Wnt-responsive PDL population effectuates extraction socket healing. J Dent Res. 97(7):803-809.
Zhang H, Chavez M B, Kolli T N, Tan M H, Fong H, Chu E Y, Li Y, Ren X, Watanabe K, Kim D G, et al. 2020. Dentoalveolar defects in the Hyp mouse model of X-linked hypophosphatemia. J Dent Res. 99(4):419-428.
Zou M L, Chen Z H, Teng Y Y, Liu S Y, Jia Y, Zhang K W, Sun Z L, Wu J J, Yuan Z D, Feng Y, et al. 2021. The Smad dependent TGF-β and BMP signaling pathway in bone remodeling and therapies. Front Mol Biosci. 8:593310.
Addison W N, Azari F, Sørensen E S, Kaartinen M T, McKee M D. 2007. Pyrophosphate inhibits mineralization of osteoblast cultures by binding to mineral, up-regulating osteopontin, and inhibiting alkaline phosphatase activity. Journal of Biological Chemistry. 282(21):15872-15883.
Addison W N, Masica D L, Gray J J, McKee M D. 2009. Phosphorylation-dependent inhibition of mineralization by osteopontin asarm peptides is regulated by phex cleavage. Journal of Bone and Mineral Research. 25(4):090923081604075-090923081604044.
Ao M, Chavez M B, Chu E Y, Hemstreet K C, Yin Y, Yadav M C, Millan J L, Fisher L W, Goldberg H A, Somerman M J et al. 2017. Overlapping functions of bone sialoprotein and pyrophosphate regulators in directing cementogenesis. Bone. 105:134-147.
Asai K, Funaba M, Murakami M. 2014. Enhancement of rankl-induced mitf-e expression and osteoclastogenesis by tgf-beta. Cell Biochem Funct. 32(5):401-409.
Balooch G, Balooch M, Nalla R K, Schilling S, Filvaroff E H, Marshall G W, Marshall S J, Ritchie R O, Derynck R, Alliston T. 2005. Tgf-beta regulates the mechanical properties and composition of bone matrix. Proc Natl Acad Sci USA. 102(52):18813-18818.
Bandyopadhyay A, Tsuji K, Cox K, Harfe B D, Rosen V, Tabin C J. 2006. Genetic analysis of the roles of bmp2, bmp4, and bmp7 in limb patterning and skeletogenesis. PLoS Genet. 2(12):e216.
Barros N M, Hoac B, Neves R L, Addison W N, Assis D M, Murshed M, Carmona A K, McKee M D. 2013. Proteolytic processing of osteopontin by phex and accumulation of osteopontin fragments in hyp mouse bone, the murine model of x-linked hypophosphatemia. J Bone Miner Res. 28(3):688-699.
Blissett A R, Garbellini D, Calomeni E P, Mihai C, Elton T S, Agarwal G. 2009. Regulation of collagen fibrillogenesis by cell-surface expression of kinase dead ddr2. J Mol Biol. 385(3):902-911.
Bonilla-Claudio M, Wang J, Bai Y, Klysik E, Selever J, Martin J F. 2012. Bmp signaling regulates a dose-dependent transcriptional program to control facial skeletal development. Development. 139(4):709-719.
Boskey A L, Spevak L, Paschalis E, Doty S B, McKee M D. 2002. Osteopontin deficiency increases mineral content and mineral crystallinity in mouse bone. Calcif Tissue Int. 71(2):145-154.
Boudiffa M, Wade-Gueye N M, Guignandon A, Vanden-Bossche A, Sabido O, Aubin J E, Jurdic P, Vico L, Lafage-Proust M H, Malaval L. 2010. Bone sialoprotein deficiency impairs osteoclastogenesis and mineral resorption in vitro. J Bone Miner Res. 25(12):2669-2679.
Boukpessi T, Hoac B, Coyac B R, Leger T, Garcia C, Wicart P, Whyte M P, Glorieux F H, Linglart A, Chaussain C et al. 2017. Osteopontin and the dento-osseous pathobiology of x-linked hypophosphatemia. Bone. 95:151-161.
Bouleftour W, Boudiffa M, Wade-Gueye N M, Bouet G, Cardelli M, Laroche N, Vanden-Bossche A, Thomas M, Bonnelye E, Aubin J E et al. 2014. Skeletal development of mice lacking bone sialoprotein (bsp)—impairment of long bone growth and progressive establishment of high trabecular bone mass. PLoS One. 9(5):e95144.
Bouxsein M L, Boyd S K, Christiansen B A, Guldberg R E, Jepsen K J, Muller R. 2010. Guidelines for assessment of bone microstructure in rodents using micro-computed tomography. J Bone Miner Res. 25(7):1468-1486.
Boyan B D, Olivares-Navarrete R, Berger M B, Hyzy S L, Schwartz Z. 2018. Role of wnt11 during osteogenic differentiation of human mesenchymal stem cells on microstructured titanium surfaces. Sci Rep. 8(1):8588.
Boyce B F, Xing L. 2008. Functions of rankl/rank/opg in bone modeling and remodeling. Arch Biochem Biophys. 473(2):139-146.
Buss D J, Reznikov N, McKee M D. 2020. Crossfibrillar mineral tessellation in normal and hyp mouse bone as revealed by 3d fib-SEM microscopy. J Struct Biol. 212(2):107603.
Caetano da Silva C, Edouard T, Fradin M, Aubert-Mucca M, Ricquebourg M, Raman R, Salles J P, Charon V, Guggenbuhl P, Muller M et al. 2022. Wnt11, a new gene associated with early onset osteoporosis, is required for osteoblastogenesis. Hum Mol Genet. 31(10):1622-1634.
Carmeliet P, Jain R K. 2011. Molecular mechanisms and clinical applications of angiogenesis. Nature. 473(7347):298-307.
Chavez M B, Chu E Y, Kram V, de Castro L F, Somerman M J, Foster B L. 2021. Guidelines for micro-computed tomography analysis of rodent dentoalveolar tissues. JBMR Plus. 5(3):e10474.
Chavez M B, Kolli T N, Tan M H, Zachariadou C, Wang C, Embree M C, Lira Dos Santos E J, Nociti F H, Jr., Wang Y, Tatakis D N et al. 2019. Loss of discoidin domain receptor 1 predisposes mice to periodontal breakdown. J Dent Res. 98(13):1521-1531.
Chim S M, Tickner J, Chow S T, Kuek V, Guo B, Zhang G, Rosen V, Erber W, Xu J. 2013. Angiogenic factors in bone local environment. Cytokine Growth Factor Rev. 24(3):297-310.
Chou L Y, Chen C H, Chuang S C, Cheng T L, Lin Y H, Chou H C, Fu Y C, Wang Y H, Wang C Z. 2020. Discoidin domain receptor 1 regulates runx2 during osteogenesis of osteoblasts and promotes bone ossification via phosphorylation of p38. Int J Mol Sci. 21(19).
Colavite P M, Vieira A E, Palanch Repeke C E, de Araujo Linhari R P, De Andrade R, Borrego A, De Franco M, Trombone A P F, Garlet G P. 2019. Alveolar bone healing in mice genetically selected in the maximum (airmax) or minimum (airmin) inflammatory reaction. Cytokine. 114:47-60.
Dai J, Rabie A B. 2007. Vegf: An essential mediator of both angiogenesis and endochondral ossification. J Dent Res. 86(10):937-950.
Dallas S L, Prideaux M, Bonewald L F. 2013. The osteocyte: An endocrine cell . . . And more. Endocr Rev. 34(5):658-690.
Daluiski A, Engstrand T, Bahamonde M E, Gamer L W, Agius E, Stevenson S L, Cox K, Rosen V, Lyons K M. 2001. Bone morphogenetic protein-3 is a negative regulator of bone density. Nat Genet. 27(1):84-88.
Delgado-Calle J, Sato A Y, Bellido T. 2017. Role and mechanism of action of sclerostin in bone. Bone. 96:29-37.
Docheva D, Popov C, Alberton P, Aszodi A. 2014. Integrin signaling in skeletal development and function. Birth Defects Res C Embryo Today. 102(1):13-36.
Dougall W C, Glaccum M, Charrier K, Rohrbach K, Brasel K, De Smedt T, Daro E, Smith J, Tometsko M E, Maliszewski C R et al. 1999. Rank is essential for osteoclast and lymph node development. Genes Dev. 13(18):2412-2424.
Eicher E M, Southard J L, Scriver C R, Glorieux F H. 1976. Hypophosphatemia: Mouse model for human familial hypophosphatemic (vitamin d-resistant) rickets. Proc Natl Acad Sci USA. 73(12):4667-4671.
Fedarko N S, Jain A, Karadag A, Fisher L W. 2004. Three small integrin binding ligand n-linked glycoproteins (siblings) bind and activate specific matrix metalloproteinases. FASEB J. 18(6):734-736.
Fisher L W, Fedarko N S. 2003. Six genes expressed in bones and teeth encode the current members of the sibling family of proteins. Connect Tissue Res. 44 Suppl 1:33-40.
Flynn L A, Blissett A R, Calomeni E P, Agarwal G. 2010. Inhibition of collagen fibrillogenesis by cells expressing soluble extracellular domains of ddr1 and ddr2. J Mol Biol. 395(3):533-543.
Foster B L, Ao M, Salmon C R, Chavez M B, Kolli T N, Tran A B, Chu E Y, Kantovitz K R, Yadav M, Narisawa S et al. 2018. Osteopontin regulates dentin and alveolar bone development and mineralization. Bone. 107:196-207.
Foster B L, Ao M, Willoughby C, Soenjaya Y, Holm E, Lukashova L, Tran A B, Wimer H F, Zerfas P M, Nociti F H, Jr. et al. 2015. Mineralization defects in cementum and craniofacial bone from loss of bone sialoprotein. Bone. 78:150-164.
Foster B L, Nociti F H, Jr., Somerman M J. 2014. The rachitic tooth. Endocr Rev. 35(1):1-34.
Foster B L, Soenjaya Y, Nociti F H, Jr., Holm E, Zerfas P M, Wimer H F, Holdsworth D W, Aubin J E, Hunter G K, Goldberg H A et al. 2013. Deficiency in acellular cementum and periodontal attachment in bsp null mice. J Dent Res. 92(2):166-172.
Friedman M S, Oyserman S M, Hankenson K D. 2009. Wnt11 promotes osteoblast maturation and mineralization through r-spondin 2. J Biol Chem. 284(21):14117-14125.
Ge C, Wang Z, Zhao G, Li B, Liao J, Sun H, Franceschi R T. 2016. Discoidin receptor 2 controls bone formation and marrow adipogenesis. J Bone Miner Res. 31(12):2193-2203.
Gowen L C, Petersen D N, Mansolf A L, Qi H, Stock J L, Tkalcevic G T, Simmons H A, Crawford D T, Chidsey-Frink K L, Ke H Z et al. 2003. Targeted disruption of the osteoblast/osteocyte factor 45 gene (of45) results in increased bone formation and bone mass. J Biol Chem. 278(3):1998-2007.
Harmey D, Hessle L, Narisawa S, Johnson K A, Terkeltaub R, Millan J L. 2004. Concerted regulation of inorganic pyrophosphate and osteopontin by akp2, enpp1, and ank: An integrated model of the pathogenesis of mineralization disorders. Am J Pathol. 164(4):1199-1209.
Harmey D, Johnson K A, Zelken J, Camacho N P, Hoylaerts M F, Noda M, Terkeltaub R, Millan J L. 2006. Elevated skeletal osteopontin levels contribute to the hypophosphatasia phenotype in akp2(−/−) mice. J Bone Miner Res. 21(9):1377-1386.
Haycraft C J, Serra R. 2008. Cilia involvement in patterning and maintenance of the skeleton. Curr Top Dev Biol. 85:303-332.
Ho A M, Johnson M D, Kingsley D M. 2000. Role of the mouse ank gene in control of tissue calcification and arthritis. Science. 289(5477):265-270.
Hojo H, Ohba S, Yano F, Saito T, Ikeda T, Nakajima K, Komiyama Y, Nakagata N, Suzuki K, Takato T et al. 2012. Gli1 protein participates in hedgehog-mediated specification of osteoblast lineage during endochondral ossification. J Biol Chem. 287(21):17860-17869.
Hollberg K, Hultenby K, Hayman A, Cox T, Andersson G. 2002. Osteoclasts from mice deficient in tartrate-resistant acid phosphatase have altered ruffled borders and disturbed intracellular vesicular transport. Exp Cell Res. 279(2):227-238.
Houschyar K S, Tapking C, Borrelli M R, Popp D, Duscher D, Maan Z N, Chelliah M P, Li J, Harati K, Wallner C et al. 2018. Wnt pathway in bone repair and regeneration-what do we know so far. Front Cell Dev Biol. 6:170.
Hunter G K, Hauschka P V, Poole A R, Rosenberg L C, Goldberg H A. 1996. Nucleation and inhibition of hydroxyapatite formation by mineralized tissue proteins. Biochem J. 317 (Pt 1):59-64.
Ikeda K, Takeshita S. 2016. The role of osteoclast differentiation and function in skeletal homeostasis. J Biochem. 159(1):1-8.
Inoue K, Mikuni-Takagaki Y, Oikawa K, Itoh T, Inada M, Noguchi T, Park J S, Onodera T, Krane S M, Noda M et al. 2006. A crucial role for matrix metalloproteinase 2 in osteocytic canalicular formation and bone metabolism. J Biol Chem. 281(44):33814-33824.
Joeng K S, Long F. 2013. Constitutive activation of gli2 impairs bone formation in postnatal growing mice. PLoS One. 8(1):e55134.
Karsenty G. 2017. Update on the biology of osteocalcin. Endocr Pract. 23(10):1270-1274.
Katoh M. 2008. Wnt signaling in stem cell biology and regenerative medicine. Curr Drug Targets. 9(7):565-570.
Kawano H, Sato T, Yamada T, Matsumoto T, Sekine K, Watanabe T, Nakamura T, Fukuda T, Yoshimura K, Yoshizawa T et al. 2003. Suppressive function of androgen receptor in bone resorption. Proc Natl Acad Sci USA. 100(16):9416-9421.
Kitaura Y, Hojo H, Komiyama Y, Takato T, Chung U I, Ohba S. 2014. Gli1 haploinsufficiency leads to decreased bone mass with an uncoupling of bone metabolism in adult mice. PLoS One. 9(10):e109597.
Koh J T, Zhao Z, Wang Z, Lewis I S, Krebsbach P H, Franceschi R T. 2008. Combinatorial gene therapy with bmp2/7 enhances cranial bone regeneration. J Dent Res. 87(9):845-849.
Komori T. 2020. Functions of osteocalcin in bone, pancreas, testis, and muscle. Int J Mol Sci. 21(20).
Kong Y Y, Yoshida H, Sarosi I, Tan H L, Timms E, Capparelli C, Morony S, Oliveira-dos-Santos A J, Van G, Itie A et al. 1999. Opgl is a key regulator of osteoclastogenesis, lymphocyte development and lymph-node organogenesis. Nature. 397(6717):315-323.
Kurotaki D, Yoshida H, Tamura T. 2020. Epigenetic and transcriptional regulation of osteoclast differentiation. Bone. 138:115471.
Lane N E, Yao W, Nakamura M C, Humphrey M B, Kimmel D, Huang X, Sheppard D, Ross F P, Teitelbaum S L. 2005. Mice lacking the integrin beta5 subunit have accelerated osteoclast maturation and increased activity in the estrogen-deficient state. J Bone Miner Res. 20(1):58-66.
Leucht P, Minear S, Ten Berge D, Nusse R, Helms J A. 2008. Translating insights from development into regenerative medicine: The function of wnts in bone biology. Semin Cell Dev Biol. 19(5):434-443.
Levi B, James A W, Nelson E R, Brugmann S A, Sorkin M, Manu A, Longaker M T. 2011. Role of indian hedgehog signaling in palatal osteogenesis. Plast Reconstr Surg. 127(3):1182-1190.
Lira Dos Santos E J, Chavez M B, Tan M H, Mohamed F F, Kolli T N, Foster B L, Liu E S. 2021. Effects of active vitamin d or fgf23 antibody on hyp mice dentoalveolar tissues. J Dent Res.220345211011041.
Liu Y, Li Z, Arioka M, Wang L, Bao C, Helms J A. 2019. Wnt3a accelerates delayed alveolar bone repair in ovariectomized mice. Osteoporos Int. 30(9):1873-1885.
Maeda Y, Nakamura E, Nguyen M T, Suva L J, Swain F L, Razzaque M S, Mackem S, Lanske B. 2007. Indian hedgehog produced by postnatal chondrocytes is essential for maintaining a growth plate and trabecular bone. Proc Natl Acad Sci USA. 104(15):6382-6387.
Malaval L, Wade-Gueye N M, Boudiffa M, Fei J, Zirngibl R, Chen F, Laroche N, Roux J P, Burt-Pichat B, Duboeuf F et al. 2008. Bone sialoprotein plays a functional role in bone formation and osteoclastogenesis. J Exp Med. 205(5):1145-1153.
Manrique N, Pereira C C, Luvizuto E R, Sanchez Mdel P, Okamoto T, Okamoto R, Sumida D H, Antoniali C. 2015. Hypertension modifies opg, rank, and rankl expression during the dental socket bone healing process in spontaneously hypertensive rats. Clin Oral Investig. 19(6):1319-1327.
Maruyama M, Rhee C, Utsunomiya T, Zhang N, Ueno M, Yao Z, Goodman S B. 2020. Modulation of the inflammatory response and bone healing. Front Endocrinol (Lausanne). 11:386.
Mihai C, Iscru D F, Druhan L J, Elton T S, Agarwal G. 2006. Discoidin domain receptor 2 inhibits fibrillogenesis of collagen type 1. J Mol Biol. 361(5):864-876.
Minear S, Leucht P, Jiang J, Liu B, Zeng A, Fuerer C, Nusse R, Helms J A. 2010. Wnt proteins promote bone regeneration. Sci Transl Med. 2(29):29ra30.
Mohamed F F, Ge C, Binrayes A, Franceschi R T. 2020. The role of discoidin domain receptor 2 in tooth development. J Dent Res. 99(2):214-222.
Moon Y J, Yun C Y, Choi H, Ka S O, Kim J R, Park B H, Cho E S. 2016. Smad4 controls bone homeostasis through regulation of osteoblast/osteocyte viability. Exp Mol Med. 48(9):e256.
Mosig R A, Dowling O, DiFeo A, Ramirez M C, Parker I C, Abe E, Diouri J, Aqeel A A, Wylie J D, Oblander S A et al. 2007. Loss of mmp-2 disrupts skeletal and craniofacial development and results in decreased bone mineralization, joint erosion and defects in osteoblast and osteoclast growth. Hum Mol Genet. 16(9):1113-1123.
Nagasaki A, Nagasaki K, Chu E Y, Kear B D, Tadesse W D, Ferebee S E, Li L, Foster B L, Somerman M J. 2021. Ablation of pyrophosphate regulators promotes periodontal regeneration. J Dent Res. 100(6):639-647.
Ohba S. 2020. Hedgehog signaling in skeletal development: Roles of indian hedgehog and the mode of its action. Int J Mol Sci. 21(18).
Olivares-Navarrete R, Lee E M, Smith K, Hyzy S L, Doroudi M, Williams J K, Gall K, Boyan B D, Schwartz Z. 2017. Substrate stiffness controls osteoblastic and chondrocytic differentiation of mesenchymal stem cells without exogenous stimuli. PLoS One. 12(1):e0170312.
Pampena D A, Robertson K A, Litvinova O, Lajoie G, Goldberg H A, Hunter G K. 2004. Inhibition of hydroxyapatite formation by osteopontin phosphopeptides. Biochem J. 378 (Pt 3):1083-1087.
Pang P, Shimo T, Takada H, Matsumoto K, Yoshioka N, Ibaragi S, Sasaki A. 2015. Expression pattern of sonic hedgehog signaling and calcitonin gene-related peptide in the socket healing process after tooth extraction. Biochem Biophys Res Commun. 467(1):21-26.
Rakian A, Yang W C, Gluhak-Heinrich J, Cui Y, Harris M A, Villarreal D, Feng J Q, Macdougall M, Harris S E. 2013. Bone morphogenetic protein-2 gene controls tooth root development in coordination with formation of the periodontium. Int J Oral Sci. 5(2):75-84.
Razzaque M S, Soegiarto D W, Chang D, Long F, Lanske B. 2005. Conditional deletion of indian hedgehog from collagen type 2alpha1-expressing cells results in abnormal endochondral bone formation. J Pathol. 207(4):453-461.
Regard J B, Zhong Z, Williams B O, Yang Y. 2012. Wnt signaling in bone development and disease: Making stronger bone with wnts. Cold Spring Harb Perspect Biol. 4(12).
Ripamonti U, Ferretti C, Teare J, Blann L. 2009. Transforming growth factor-beta isoforms and the induction of bone formation: Implications for reconstructive craniofacial surgery. J Craniofac Surg. 20(5):1544-1555.
Ripamonti U, Petit J C. 2009. Bone morphogenetic proteins, cementogenesis, myoblastic stem cells and the induction of periodontal tissue regeneration. Cytokine Growth Factor Rev. 20(5-6):489-499.
Ripamonti U, Teare J, Petit J C. 2006. Pleiotropism of bone morphogenetic proteins: From bone induction to cementogenesis and periodontal ligament regeneration. J Int Acad Periodontol. 8(1):23-32.
Rodrigues T L, Nagatomo K J, Foster B L, Nociti F H, Somerman M J. 2011. Modulation of phosphate/pyrophosphate metabolism to regenerate the periodontium: A novel in vivo approach. J Periodontol. 82(12):1757-1766.
Rosen V. 2006. Bmp and bmp inhibitors in bone. Ann N Y Acad Sci. 1068:19-25.
Ruchon A F, Tenenhouse H S, Marcinkiewicz M, Siegfried G, Aubin J E, DesGroseillers L, Crine P, Boileau G. 2000. Developmental expression and tissue distribution of phex protein: Effect of the hyp mutation and relationship to bone markers. J Bone Miner Res. 15(8):1440-1450.
Rutsch F, Ruf N, Vaingankar S, Toliat M R, Suk A, Hohne W, Schauer G, Lehmann M, Roscioli T, Schnabel D et al. 2003. Mutations in enpp1 are associated with ‘idiopathic’ infantile arterial calcification. Nat Genet. 34(4):379-381.
Saftig P, Hunziker E, Everts V, Jones S, Boyde A, Wehmeyer O, Suter A, von Figura K. 2000. Functions of cathepsin k in bone resorption. Lessons from cathepsin k deficient mice. Adv Exp Med Biol. 477:293-303.
Salazar V S, Zarkadis N, Huang L, Norris J, Grimston S K, Mbalaviele G, Civitelli R. 2013a. Embryonic ablation of osteoblast smad4 interrupts matrix synthesis in response to canonical wnt signaling and causes an osteogenesis-imperfecta-like phenotype. J Cell Sci. 126(Pt 21):4974-4984.
Salazar V S, Zarkadis N, Huang L, Watkins M, Kading J, Bonar S, Norris J, Mbalaviele G, Civitelli R. 2013b. Postnatal ablation of osteoblast smad4 enhances proliferative responses to canonical wnt signaling through interactions with beta-catenin. J Cell Sci. 126(Pt 24):5598-5609.
Salmon B, Bardet C, Coyac B R, Baroukh B, Naji J, Rowe P S, Opsahl Vital S, Linglart A, McKee M D, Chaussain C. 2014. Abnormal osteopontin and matrix extracellular phosphoglycoprotein localization, and odontoblast differentiation, in x-linked hypophosphatemic teeth. Connect Tissue Res. 55 Suppl 1:79-82.
Schupbach D, Comeau-Gauthier M, Harvey E, Merle G. 2020. Wnt modulation in bone healing. Bone. 138:115491.
Serra R. 2008. Role of intraflagellar transport and primary cilia in skeletal development. Anat Rec (Hoboken). 291(9):1049-1061.
Shi Y, He G, Lee W C, Mckenzie J A, Silva M J, Long F. 2017. Gli1 identifies osteogenic progenitors for bone formation and fracture repair. Nat Commun. 8(1):2043.
Soenjaya Y, Foster B L, Nociti F H, Jr., Ao M, Holdsworth D W, Hunter G K, Somerman M J, Goldberg H A. 2015. Mechanical forces exacerbate periodontal defects in bsp-null mice. J Dent Res. 94(9):1276-1285.
Tan X, Weng T, Zhang J, Wang J, Li W, Wan H, Lan Y, Cheng X, Hou N, Liu H et al. 2007. Smad4 is required for maintaining normal murine postnatal bone homeostasis. J Cell Sci. 120(Pt 13):2162-2170.
Tonniges J R, Albert B, Calomeni E P, Roy S, Lee J, Mo X, Cole S E, Agarwal G. 2016. Collagen fibril ultrastructure in mice lacking discoidin domain receptor 1. Microsc Microanal. 22(3):599-611.
Udagawa N, Takahashi N, Yasuda H, Mizuno A, Itoh K, Ueno Y, Shinki T, Gillespie M T, Martin T J, Higashio K et al. 2000. Osteoprotegerin produced by osteoblasts is an important regulator in osteoclast development and function. Endocrinology. 141(9):3478-3484.
Valverde P, Tu Q, Chen J. 2005. Bsp and rankl induce osteoclastogenesis and bone resorption synergistically. J Bone Miner Res. 20(9):1669-1679.

Vieira A E, Repeke C E, Ferreira Junior Sde B, Colavite P M, Biguetti C C, Oliveira R C, Assis G F, Taga R, Trombone A P, Garlet G P. 2015. Intramembranous bone healing process subsequent to tooth extraction in mice: Micro-computed tomography, histomorphometric and molecular characterization. PLoS One. 10(5):e0128021.
Vogel W F, Aszodi A, Alves F, Pawson T. 2001. Discoidin domain receptor 1 tyrosine kinase has an essential role in mammary gland development. Mol Cell Biol. 21(8):2906-2917.
Wade-Gueye N M, Boudiffa M, Vanden-Bossche A, Laroche N, Aubin J E, Vico L, Lafage-Proust M H, Malaval L. 2012. Absence of bone sialoprotein (bsp) impairs primary bone formation and resorption: The marrow ablation model under pth challenge. Bone. 50(5):1064-1073.
Wijgerde M, Karp S, McMahon J, McMahon A P. 2005. Noggin antagonism of bmp4 signaling controls development of the axial skeleton in the mouse. Dev Biol. 286(1):149-157.
Wu M, Chen G, Li Y P. 2016. Tgf-beta and bmp signaling in osteoblast, skeletal development, and bone formation, homeostasis and disease. Bone Res. 4:16009.
Wu X B, Li Y, Schneider A, Yu W, Rajendren G, Iqbal J, Yamamoto M, Alam M, Brunet L J, Blair H C et al. 2003. Impaired osteoblastic differentiation, reduced bone formation, and severe osteoporosis in noggin-overexpressing mice. J Clin Invest. 112(6):924-934.
Yang C, Wang C, Zhou J, Liang Q, He F, Li F, Li Y, Chen J, Zhang F, Han C et al. 2020. Fibronectin 1 activates wnt/beta-catenin signaling to induce osteogenic differentiation via integrin betal interaction. Lab Invest. 100(12):1494-1502.
Yang J, Andre P, Ye L, Yang Y Z. 2015. The hedgehog signalling pathway in bone formation. Int J Oral Sci. 7(2):73-79.
Yuan X, Pei X, Zhao Y, Tulu U S, Liu B, Helms J A. 2018. A wnt-responsive pdl population effectuates extraction socket healing. J Dent Res. 97(7):803-809.
Zhang H, Chavez M B, Kolli T N, Tan M H, Fong H, Chu E Y, Li Y, Ren X, Watanabe K, Kim D G et al. 2020a. Dentoalveolar defects in the hyp mouse model of x-linked hypophosphatemia. J Dent Res. 99(4):419-428.
Zhang Y, Su J, Wu S, Teng Y, Yin Z, Guo Y, Li J, Li K, Yao L, Li X. 2015. Ddr2 (discoidin domain receptor 2) suppresses osteoclastogenesis and is a potential therapeutic target in osteoporosis. Sci Signal. 8(369):ra31.
Zhang Z, Hu P, Wang Z, Qiu X, Chen Y. 2020b. Bdnf promoted osteoblast migration and fracture healing by up-regulating integrin betal via trkb-mediated erk1/2 and akt signalling. J Cell Mol Med. 24(18):10792-10802.
Zhao M, Zhao Z, Koh J T, Jin T, Franceschi R T. 2005. Combinatorial gene therapy for bone regeneration: Cooperative interactions between adenovirus vectors expressing bone morphogenetic proteins 2, 4, and 7. J Cell Biochem. 95(1):1-16.
Zou M L, Chen Z H, Teng Y Y, Liu S Y, Jia Y, Zhang K W, Sun Z L, Wu J J, Yuan Z D, Feng Y et al. 2021. The smad dependent tgf-beta and bmp signaling pathway in bone remodeling and therapies. Front Mol Biosci. 8:593310.

### Example 2. Exogenous Bone Sialoprotein Improves Alveolar Bone Healing in Mice

### Overview

Bone sialoprotein (Ibsp/BSP) is an extracellular matrix protein associated with mineralized tissues and shown to promote hydroxyapatite nucleation and growth. BSP includes a collagen-binding motif, 2-3 polyglutamic acid hydroxyapatite-nucleating domains, and an RGD integrin-binding sequence; post-translational modifications (PTMs) including serine phosphorylation and O/N-linked glycosylations also contribute to mineralization functions. Ibsp knockout (Ibsp^{-/-}) mice exhibit defective bone formation and remodeling, with alveolar bone showing more pronounced hypomineralization than long bones. We previously documented defects in alveolar bone healing in Ibsp^{-/-} vs. wild-type (WT) control mice. We hypothesized BSP would rescue defective alveolar bone healing in Ibsp^{-/-} mice. First maxillary molars were bilaterally extracted from 42 days postnatal Ibsp^{-/-} and WT mice. Collagen gel with or without BSP was delivered to sockets. BSP added was either native rat long bone BSP (nBSP) or recombinant rat BSP (rBSP). Bovine type I collagen remained liquid while on ice and solidified to gel at 37 °C. Tissues were harvested 0, 1, 2, 7, and 14 days post-procedure (dpp) and analyzed by micro-computed tomography (microCT), histology, and immunohistochemistry (IHC). Histology and IHC demonstrated that collagen and BSP were retained within sockets during early stages of wound healing. At 14 dpp, bone volume fraction (BV/TV) was increased by both nBSP (53%; P<0.001) and rBSP (69%; P<0.0001), compared to collagen vehicle in Ibsp^{-/-} mice. BMD of new bone was increased by nBSP (54%; P<0.0001) and rBSP (66%; P<0.0001) compared to collagen only in Ibsp^{-/-} mice. Both nBSP and rBSP normalized BV/TV and BMD in Ibsp^{-/-} mice to WT levels. Surprisingly, in WT mice, BV/TV of healing alveolar bone was also increased by nBSP (34%; P<0.001) and rBSP (65%; P<0.0001) compared to controls. BMD of new bone in WT mice was increased by nBSP (35%; P<0.0001) and rBSP (62%; P<0.0001) vs. controls. Exogenous BSP rescued alveolar bone healing defects in Ibsp^{-/-} mice and enhanced bone healing in WT mice. Equivalent effects of nBSP and rBSP suggest PTMs are not essential for BSP functions in bone healing.

### Background

Bone sialoprotein (BSP) is an extracellular matrix (ECM) protein associated with mineralized tissues, including alveolar bone (Foster et al. 2015; Ganss et al. 1999; Goldberg and Hunter 2012). BSP includes three evolutionarily conserved functional domains. An N-terminal collagen-binding domain promotes BSP-collagen interactions. Two or more (depending on species) polyglutamic acid (polyE) domains promote hydroxyapatite (HA) crystal nucleation and growth, which is also influenced by posttranslational modifications (PTMs), including serine residue phosphorylations and O- and N-linked glycosylations (Goldberg et al. 1996; Hunter and Goldberg 1994; Miwa et al. 2010; Tye et al. 2003; Xu et al. 2017). A C-terminal arginine-glycine-aspartic acid (RGD) integrin-binding domain is associated with osteoblast differentiation and function (Gordon et al. 2009; Gordon et al. 2007). While in vitro experiments have provided some insights into BSP functions, in vivo function(s) of BSP in bone biology remain unclear.

Ibsp knockout (Ibsp^{-/-}) mice featuring genetic ablation of BSP exhibit defective osteoblast and osteoclast differentiation and functions, delayed long bone mineralization, and abnormal bone remodeling (Boudiffa et al. 2010; Holm et al. 2015; Malaval et al. 2008). Impaired bone healing was reported following surgically created cortical bone defects and bone marrow ablation in Ibsp^{-/-} mouse femurs (Malaval et al. 2009b; Wade-Gueye et al. 2012). Knockout of BSP caused severe alveolar bone hypomineralization, suggesting a critical role of the protein in mineralization in the craniofacial region, possibly associated with neural crest-derived osteoprogenitor cells and/or the intramembranous mode of ossification (Ao et al. 2017; Foster et al. 2015; Foster et al. 2013). Using a mouse molar extraction and alveolar bone healing model in Ibsp^{-/-} mice, we found that BSP is critical and non-redundant for alveolar bone healing. Compared to controls, Ibsp^{-/-} mice exhibited reduced alveolar bone volume and density, persistence of woven bone, and substantial dysregulation of genes associated with osteoblast differentiation and function (Chavez et al. 2023).

Using the same mouse molar extraction and alveolar bone healing model, we tested whether exogenous BSP could promote improved alveolar bone healing in Ibsp^{-/-} and control mice. Further, we compared native BSP (isolated from rat long bones) to recombinant BSP (derived from bacterial expression vector, therefore lacking PTMs) to test whether PTMs were important to the role of BSP in bone healing. We hypothesized BSP would rescue defective alveolar bone healing in Ibsp^{-/-} mice. We used a multimodal approach including high resolution micro-computed tomography, histology, and immunohistochemistry, to determine the capability of BSP to promote alveolar bone healing.

### Materials and Methods

Mice. Animal studies were approved by the Ohio State University Institutional Animal Care and Use Committee (protocol #2015A00000078-R2) and followed ARRIVE guidelines 2.0. Ibsp knockout (Ibsp^{-/-}) mice have been previously characterized (Foster et al. 2015; Foster et al. 2013). A model of mouse maxillary first molar (M1) extraction and socket healing was previously described (Chavez et al. 2023). Alveolar bone healing was compared in 42 days postnatal (dpn) Ibsp^{-/-} vs. wild-type (WT; Ibsp^{+/+}) littermate control mice on a mixed 129/CD1 background (n=4-9 mice/genotype, including both males and females). Mice were anesthetized by nasal isoflurane and bilateral extraction of maxillary M1 was accomplished using a #2 dental explorer and micro-jaw Castroviejo needle holder under a surgical microscope. After extraction, blood flow from the socket was staunched using sterile gauze and paper points. Liquid type I collagen solution was administered by 28 gauge tuberculin syringe into empty sockets with or without 1 mg/ml native BSP (nBSP) or 1 mg/ml recombinant BSP (rBSP). Preparations of nBSP and rBSP are described below.

After the extraction, mice received 2 mg/kg meloxicam and were provided hydrogel and softened food for 3 days post procedure (dpp), and weight was monitored during healing. Criteria for animal removal from the study included 20% or more weight loss from baseline body weight, body condition scoring indicating illness, or infection of surgical wounds deemed untreatable by veterinary staff. Over the course of the study, n=38 mice underwent extraction, and none were removed from the study due to postsurgical surgical complications. Weight loss less than 10% of body weight was typically observed at 1 dpp. Mice quickly recovered to starting weight or above by 3 dpp. Upon regaining consciousness, mice returned to normal behavior of eating, drinking, and grooming within minutes.

Tissues were harvested at 0, 1, 2, 7 and 14 dpp and fixed in 10% formalin for 24 hours. Healing of epithelia and retention of collagen gel and BSP were examined at 0, 1, 2, and 7 dpp by histology (described below). Tissues at 0 and 14 dpp were analyzed by micro-computed tomography to determine bone healing (described below).

Bone Sialoprotein Preparation. Native rat BSP (nBSP) was purified from the long bones of adult rats as described previously (Goldberg and Sodek 1994; Tye et al. 2003). Fractionation of BSP was accomplished by repeated ion exchange chromatography, and in part by gel filtration and hydroxyapatite chromatography. Preparation of recombinant rat BSP (rBSP) has been described previously (Tye et al. 2003). Full-length rat BSP with a thrombin-cleavable pentahistidine (5×His) tail was cloned into an expression plasmid and transformed into E. coli strain BL21 (DE3). Protein extracts from bacterial cultures were purified to 99%+ purity by column chromatography employing histidine-binding resin and then fast protein liquid chromatography (FPLC) purification.

Collagen Gel Preparation. BSP-laden collagen gel was prepared on ice to prevent collagen from gelling before administration to sockets, as described previously (Yuan et al. 2018). This collagenous mixture is liquid at 4 °C and assumes a gel-like consistency at 37 °C, aiding in its retention within the socket. nBSP or rBSP was resuspended in 10× phosphate buffered saline (PBS) diluted with sterile saline to 1× and added to 5 mg/ml bovine type I collagen solution (Gibco, ThermoFisher Scientific, Waltham, MA USA). IN NaOH was used to adjust the pH to 7.2.

Micro-computed Tomography (MicroCT). Samples were scanned in a μCT 50 (Scanco Medical, Bassersdorf, Switzerland) at 70 kVp, 76 μA, 0.5 mm Al filter, with 900-ms integration and 6-μm voxel dimension. DICOM files were created from raw data, exported, and calibrated to a standard curve calculated from five known densities of hydroxyapatite (mg/cm³ HA). Reconstructed images were loaded and analyzed using Analyze 14.0 (AnalyzeDirect, Overland Park, KS) as previously described using a sample registration approach to define the region of interest (ROI) based on an average M1 socket volume (Chavez et al. 2023). Tissue above 650 mg/cm³ HA was defined as bone and used to calculate bone volume fraction (BV/TV), bone mineral density (BMD), and tissue mineral density (TMD) (Bouxsein et al. 2010; Chavez et al. 2021).

Histology. Tissues for histology were decalcified in an acetic acid/formalin/sodium chloride (AFS) solution, and paraffin embedded for 6 μm sagittal sections (Foster 2012). Histological staining included hematoxylin and eosin (H&E), Masson's trichrome (MT), and picrosirius red (PR) stains. Immunohistochemistry (IHC) was performed with an avidin-biotinylated peroxidase kit with 3-amino-9-ethylcarbazole substrate (Vector Labs, Burlingame, CA, USA). Primary antibodies used were LF-68 rabbit polyclonal anti-collagen type I alpha I (COL1A1) IgG (provided Dr. Larry Fisher, National Institute of Dental and Craniofacial Research [NIDCR], Bethesda, MD, USA) (Chavez et al. 2023), and LF-175 rabbit polyclonal anti-osteopontin (OPN) IgG (provided Dr. Larry Fisher, NIDCR) (Chavez et al. 2023; Foster et al. 2018).

Statistical Analyses. Mean±standard deviation (SD) are shown in graphs (* P<0.05; ** P<0.01; *** P≤0.001; **** P≤0.0001). Two-way ANOVA with post-hoc Tukey's multiple comparisons test were performed with GraphPad Prism v9.0.2 (San Diego, CA).

### Results

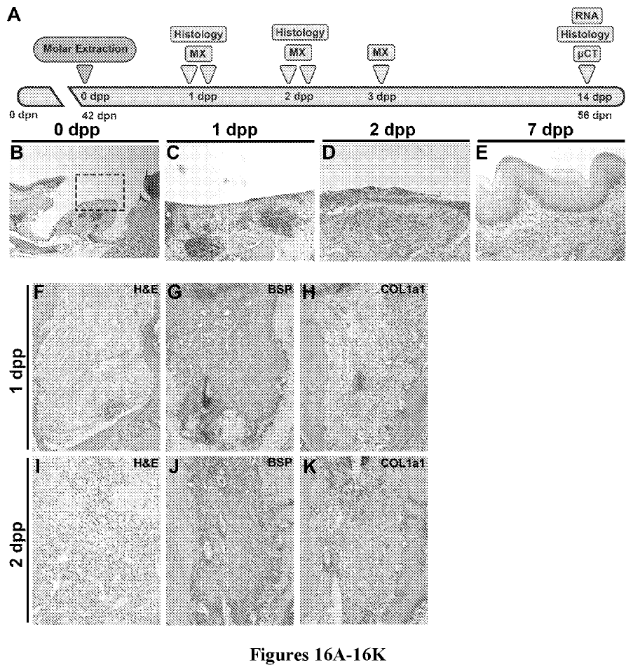

BSP and Collagen are Retained in Maxillary Molar Sockets. Ibsp^{-/-} mice exhibit severely defective alveolar bone healing marked by reduced bone volume and density, persistence of woven bone with large marrow spaces, and dysregulation of genes associated with bone cell functions and mineralization (Chavez et al. 2023). We delivered exogenous native BSP (nBSP) or recombinant BSP (rBSP) in collagen gel at the time of maxillary first molar extraction. Molars were extracted at 42 dpn and the primary endpoint was 14 days post-procedure (dpp). Histology was performed at 1, 2, and 14 dpp (FIG. 16A).

Histology showed that epithelial healing occurred rapidly after maxillary molar extraction. Extraction sockets showed closure and presence of granulation tissue by 1 dpp (FIG. 16B, 16C). Epithelialization was evident by 2 dpp and full thickness oral epithelium was present by 7 dpp (FIG. 16D, 16E). IHC in Ibsp^{-/-} mouse alveolar bone demonstrated that at 1 and 2 dpp, both collagen gel and BSP were detectable within the operated sockets (FIG. 16F-16K).

Histological observations confirmed retention of BSP and collagen during key early stages of wound closure and epithelial repair.

Exogenous BSP Improves Alveolar Bone Healing in WT and Ibsp Knockout Mice. The most active alveolar bone repair in WT and Ibsp^{-/-} mice occurs between 7 and 14 dpp; by 21 dpp, the majority of healing is completed in both genotypes, with little addition of bone quality or quantity by 56 dpp (Chavez. et al. 2023). In order to test for effects of exogenous BSP, we focused on 14 dpp as a stage of active bone repair in terms of tissue changes, cell activities, and gene expression.

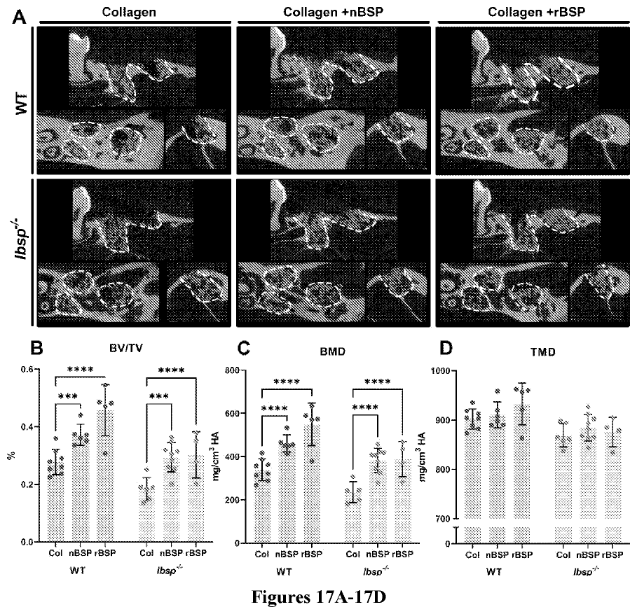

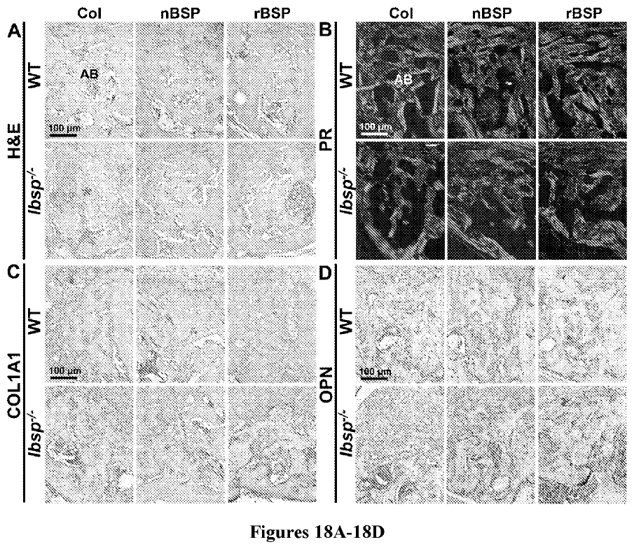

At 14 dpp, both genotypes and all treatment groups showed evidence of bone healing (FIG. 17A). Our first observation was that Ibsp^{-/-} mice administered collagen with either nBSP or rBSP showed improved alveolar bone healing compared to those receiving carrier alone (FIG. 17B-17D). Bone volume fraction (BV/TV) of healing alveolar bone was increased by nBSP (53%; P<0.001) and rBSP (69%; P<0.0001) compared to controls. Bone mineral density (BMD) of new bone was increased by nBSP (54%; P<0.0001) and rBSP (66%; P<0.0001) compared to controls. Tissue mineral density (TMD) was not different among treatment groups in Ibsp^{-/-} mice. BSP improved BV/TV and BMD in Ibsp^{-/-} mice receiving BSP to levels equivalent to WT mice receiving only the collagen carrier.

Our second observation was that exogenous BSP also improved alveolar bone healing in WT mice (FIG. 17B-17D). BV/TV of WT healing alveolar bone was increased by nBSP (34%; P<0.001) and rBSP (65%; P<0.0001) compared to controls. BMD of new bone was increased by nBSP (35%; P<0.0001) and rBSP (62%; P<0.0001) compared to controls. TMD was not different among treatment groups in WT mice.

Our third observation was that there was no difference when comparing effects of nBSP and rBSP on alveolar bone healing in either genotype (FIG. 17B-17D). Both nBSP and rBSP improved alveolar bone healing in both genotypes with no substantial differences in bone quantity or quality.

Histology provided additional insights into alveolar bone healing at 14 dpp. H&E staining revealed progressive bone deposition in both genotypes (FIG. 18A). Sockets for all mice showed deposition of woven, disorganized bone. Ibsp^{-/-} mice showed reduced quantities of new bone compared to WT, though groups administered nBSP or rBSP showed increased bone. PR staining viewed under polarized light showed a preponderance of red fibers with presence of smaller numbers of yellow fibers (FIG. 18B). COL1A1 IHC confirmed the collagen content of the new bone in all sockets (FIG. 18C). Presence of OPN in healing bone was detected in all genotypes and experimental groups (FIG. 18D).

### Discussion

Example 1 demonstrated that BSP is critical for proper periodontal development, mineralization, and function, as well as for alveolar bone healing (Ao et al. 2017; Chavez et al. 2023; Foster et al. 2015; Foster et al. 2013). We used a mouse model of maxillary molar extraction to analyze whether exogenous BSP delivered in a collagen gel could improve alveolar bone healing in Ibsp^{-/-} and WT control mice. We confirmed collagen and BSP were retained in sockets in the early stages of healing. Exogenous BSP rescued alveolar bone healing defects in Ibsp^{-/-} mice and enhanced bone healing in WT mice. Equivalent effects of nBSP and rBSP suggest PTMs are not essential for BSP functions in bone healing. These results support BSP as an approach to improve the quantity and quality of new bone in alveolar bone healing.

The Role of BSP in Bone Formation and Healing. Genetic deletion of Ibsp/BSP impairs bone formation, mineralization, and remodeling through effects on osteoblasts and osteoclasts (Boudiffa et al. 2010; Bouleftour et al. 2014; Holm et al. 2015; Malaval et al. 2009a; Malaval et al. 2008). We demonstrated that alveolar bone mineralization is arguably more severely defective than postcranial skeletal defects, with accumulation of large swaths of osteoid and subsequent periodontal breakdown (Ao et al. 2017; Foster et al. 2015; Foster et al. 2013; Soenjaya et al. 2015). This greater impact may be associated with the neural crest ectomesenchymal origin of osteoblasts of craniofacial bone as opposed to mesodermal origins of the postcranial skeleton, the intramembranous mode of ossification of alveolar bone versus endochondral ossification in long bones, or other factor(s). Genetic ablation of BSP caused defective bone healing in cortical defects introduced in femurs, as well as marrow ablation models affecting trabecular bone (Malaval et al. 2009b; Monfoulet et al. 2010; Wade-Gueye et al. 2012). Using a model of maxillary molar extraction and alveolar bone healing, we found approximately 40% reduction in alveolar bone healing in Ibsp^{-/-} vs. WT mice characterized by reduced bone quality and quantity, disorganized bone with the appearance of woven bone, large persistent marrow spaces, and dysregulated cell signaling (Chavez et al. 2023). We used this extraction model to analyze effects of exogenous BSP delivery on alveolar bone healing.

Few studies have tested delivery of exogenous BSP to healing bone defects. Exogenous bovine bone BSP cross-linked to rat type I collagen promoted increased calcification in critical size rat calvarial defects but did not promote calcification at subcutaneous sites (Wang et al. 2006). In another study, BSP immobilized to type I collagen and added to rat calvarial defects only marginally improved bone healing (Kriegel et al. 2022). In the same report, in a second femoral critical size defect model in rats, a polylactide cylinder filled with type I collagen and immobilized BSP induced bone regeneration. We used the straightforward approach of exogenous rat BSP combined with a carrier of collagen type I that remains liquid when cold, but gels at body temperature. This delivery system was used to successfully deliver other bioactive factors for alveolar bone healing (Yuan et al. 2018). The gelling of collagen is thought to aid in retention of the BSP within the socket during early stages of healing, as demonstrated by histology and immunostaining. We opted to not crosslink BSP with collagen because BSP harbors an evolutionarily conserved N-terminal hydrophobic collagen-binding domain that contributes to a strong interaction and may direct sites of hydroxyapatite nucleation onto collagen fibrils (Baht et al. 2008; Goldberg and Hunter 2012; Tye et al. 2005).

We compared BSP prepared from rat bones (native BSP; nBSP) to rat BSP synthesized by an expression vector in E. coli bacteria (recombinant BSP; rBSP) (Goldberg and Sodek 1994; Tye et al. 2003). nBSP harbors the full complement of PTMs, including serine phosphorylations and N- and O-linked glycosylations that are thought to contribute to its hydroxyapatite nucleating abilities (Baht et al. 2010; Goldberg and Hunter 2012; Xu et al. 2017). In contrast, rBSP produced in bacteria does not include PTMs, thus effects on cells and bone healing would be predominantly due to the primary amino acid sequence and the conserved domains, an N-terminal collagen-binding domain, polyglutamic acid (polyE) sequences (three in rats), and the C-terminal arginine-glycine-aspartic acid (RGD) integrin-binding domain. All these domains have been implicated in its functions in osteoblast differentiation and bone mineralization (Baht et al. 2008; Goldberg and Hunter 2012; Goldberg et al. 1996; Gordon et al. 2009; Gordon et al. 2007; Hunter and Goldberg 1994; Hunter et al. 1996; Tye et al. 2005; Tye et al. 2003; Wazen et al. 2007). In the molar extraction model, we found no difference in efficacy of nBSP vs. rBSP to promote improved alveolar bone healing in Ibsp^{-/-} or WT mice. These results suggest the functional domains and not the PTMs are the most critical factors in promoting bone repair. This finding may support use of rBSP in future translational and clinical studies. This is an important practical finding because the laborious and costly isolation and purification of BSP from animal bone (Goldberg and Sodek 1994) can potentially be avoided and scaling up production could be more easily achieved.

### Conclusion

These results show potential for BSP to promote alveolar bone healing. Additional research on several fronts is necessary to explore this further. To better understand the mechanism through which BSP promotes healing, additional gene engineered mouse models where functional domains are inactivated, and/or delivery of altered forms of BSP, should be considered. This approach may indicate whether collagen binding is important, if the polyE sequences are primary drivers of early mineral nucleation and growth, and/or whether the RGD sequence is influencing migration, attachment, or differentiation of osteoblasts, osteoclasts, or other cells. While our studies employed young mice for practical reasons (allowing predictable extraction of molars), BSP delivery may be an important approach to consider to improve bone healing with age. At advanced age, suboptimal healing can affect periodontal treatment outcomes and alveolar ridge maintenance for dental implant placement (Amler 1977; 1993; Chen et al. 2018; Huang et al. 2016; Liu et al. 2019; Shimizu et al. 1998; Tanaka et al. 2001; Van der Velden 1984). There may be a role for BSP in improvement of bone healing in these clinical scenarios. Exploration of these approaches is underway.

The mouse molar socket healing model has been a useful and predictable model to test in vivo functions of BSP; however, the importance of BSP for acellular cementum formation and periodontal function is equally critical (Ao et al. 2017; Foster et al. 2015; Foster et al. 2013; Soenjaya et al. 2015). Based on the ability for BSP to affect alveolar bone healing, this protein's potential to promote cementum repair and regeneration represents an exciting clinical opportunity.

It should also be considered whether BSP can be used in conjunction with other factors that promote mineralized tissue repair. We showed previously that genetic reduction of the mineralization inhibitor, inorganic pyrophosphate (PPᵢ), can ameliorate cementum and alveolar bone defects in Ibsp^{-/-} mice (Ao et al. 2017). Furthermore, pharmacologic delivery of tissue-nonspecific alkaline phosphatase (TNAP; which enzymatically reduces PPᵢ concentrations) by gene therapy or local injection into periodontal tissues was able to promote cementum and alveolar bone healing repair in Ibsp^{-/-} mice (Nagasaki et al. 2021). These findings suggest that BSP and TNAP participate in distinct ways in dentoalveolar mineralization and that approaches employing combinations of BSP and TNAP, and potentially other factors, may achieve greater results than single factor treatments.

### REFERENCES

Amler M H. 1977. The age factor in human extraction wound healing. J Oral Surg. 35(3):193-197.
Amler M H. 1993. Age factor in human alveolar bone repair. J Oral Implantol. 19(2):138-142.
Ao M, Chavez M B, Chu E Y, Hemstreet K C, Yin Y, Yadav M C, Millan J L, Fisher L W, Goldberg H A, Somerman M J et al. 2017. Overlapping functions of bone sialoprotein and pyrophosphate regulators in directing cementogenesis. Bone. 105:134-147.
Baht G S, Hunter G K, Goldberg H A. 2008. Bone sialoprotein-collagen interaction promotes hydroxyapatite nucleation. Matrix Biol. 27(7):600-608.
Baht G S, O'Young J, Borovina A, Chen H, Tye C E, Karttunen M, Lajoie, Hunter G K, Goldberg H A. 2010. Phosphorylation of ser136 is critical for potent bone sialoprotein-mediated nucleation of hydroxyapatite crystals. Biochem J. 428(3):385-395.
Boudiffa M, Wade-Gueye N M, Guignandon A, Vanden-Bossche A, Sabido O, Aubin J E, Jurdic P, Vico L, Lafage-Proust M H, Malaval L. 2010. Bone sialoprotein deficiency impairs osteoclastogenesis and mineral resorption in vitro. J Bone Miner Res. 25(12):2669-2679.
Bouleftour W, Boudiffa M, Wade-Gueye N M, Bouet G, Cardelli M, Laroche N, Vanden-Bossche A, Thomas M, Bonnelye E, Aubin J E et al. 2014. Skeletal development of mice lacking bone sialoprotein (bsp)—impairment of long bone growth and progressive establishment of high trabecular bone mass. PLoS One. 9(5):e95144.
Bouxsein M L, Boyd S K, Christiansen B A, Guldberg R E, Jepsen K J, Muller R. 2010. Guidelines for assessment of bone microstructure in rodents using micro-computed tomography. J Bone Miner Res. 25(7):1468-1486.
Chavez M B, Chu E Y, Kram V, de Castro L F, Somerman M J, Foster B L. 2021. Guidelines for micro-computed tomography analysis of rodent dentoalveolar tissues. JBMR Plus. 5(3):e10474.
Chavez M B, Tan M H, Kolli T N, Zachariadou C, Farah F, Mohamed F F, Chu E Y, Foster B L. 2023. Bone sialoprotein is critical for alveolar bone healing in mice. Journal of Dental Research. 102(2):187-196.
Chen C H, Wang L, Serdar Tulu U, Arioka M, Moghim M M, Salmon B, Chen C T, Hoffmann W, Gilgenbach J, Brunski J B et al. 2018. An osteopenic/osteoporotic phenotype delays alveolar bone repair. Bone. 112:212-219.
Foster B L. 2012. Methods for studying tooth root cementum by light microscopy. Int J Oral Sci. 4(3):119-128.
Foster B L, Ao M, Salmon C R, Chavez M B, Kolli T N, Tran A B, Chu E Y, Kantovitz K R, Yadav M, Narisawa S et al. 2018. Osteopontin regulates dentin and alveolar bone development and mineralization. Bone. 107:196-207.
Foster B L, Ao M, Willoughby C, Soenjaya Y, Holm E, Lukashova L, Tran A B, Wimer H F, Zerfas P M, Nociti F H, Jr. et al. 2015. Mineralization defects in cementum and craniofacial bone from loss of bone sialoprotein. Bone. 78:150-164.
Foster B L, Soenjaya Y, Nociti F H, Jr., Holm E, Zerfas P M, Wimer H F, Holdsworth D W, Aubin J E, Hunter G K, Goldberg H A et al. 2013. Deficiency in acellular cementum and periodontal attachment in bsp null mice. J Dent Res. 92(2):166-172.
Ganss B, Kim R H, Sodek J. 1999. Bone sialoprotein. Crit Rev Oral Biol Med. 10(1):79-98.
Goldberg H A, Hunter G K. 2012. Functional domains of bone sialoprotein. In: Goldberg M, editor. Phosphorylated extracellular matrix proteins of bone and dentin. Bentham Science Publishers. p. 266-282.
Goldberg H A, Sodek J. 1994. Purification of mineralized tissue-associated osteopontin. J Tissue Cult Methods. 16(3-4):211-215.
Goldberg H A, Warner K J, Stillman M J, Hunter G K. 1996. Determination of the hydroxyapatite-nucleating region of bone sialoprotein. Connect Tissue Res. 35(1-4):385-392.
Gordon J A, Hunter G K, Goldberg H A. 2009. Activation of the mitogen-activated protein kinase pathway by bone sialoprotein regulates osteoblast differentiation. Cells Tissues Organs. 189(1-4):138-143.
Gordon J A, Tye C E, Sampaio A V, Underhill T M, Hunter G K, Goldberg H A. 2007. Bone sialoprotein expression enhances osteoblast differentiation and matrix mineralization in vitro. Bone. 41(3):462-473.
Holm E, Aubin J E, Hunter G K, Beier F, Goldberg H A. 2015. Loss of bone sialoprotein leads to impaired endochondral bone development and mineralization. Bone. 71:145-154.
Huang L, Salmon B, Yin X, Helms J A. 2016. From restoration to regeneration: Periodontal aging and opportunities for therapeutic intervention. Periodontol 2000. 72(1):19-29.
Hunter G K, Goldberg H A. 1994. Modulation of crystal formation by bone phosphoproteins: Role of glutamic acid-rich sequences in the nucleation of hydroxyapatite by bone sialoprotein. Biochem J. 302 (Pt 1)(Pt 1):175-179.
Hunter G K, Hauschka P V, Poole A R, Rosenberg L C, Goldberg H A. 1996. Nucleation and inhibition of hydroxyapatite formation by mineralized tissue proteins. Biochem J. 317 (Pt 1)(Pt 1):59-64.
Kriegel A, Schlosser C, Habeck T, Dahmen C, Gotz H, Clauder F, Armbruster F P, Baranowski A, Drees P, Rommens P M et al. 2022. Bone sialoprotein immobilized in collagen type i enhances bone regeneration in vitro and in vivo. Int J Bioprint. 8(3):591.
Liu Y, Li Z, Arioka M, Wang L, Bao C, Helms J A. 2019. Wnt3a accelerates delayed alveolar bone repair in ovariectomized mice. Osteoporos Int. 30(9):1873-1885.
Malaval L, Aubin J E, Vico L. 2009a. Role of the small integrin-binding ligand n-linked glycoprotein (sibling), bone sialoprotein (bsp) in bone development and remodeling. Osteoporos Int. 20(6):1077-1080.
Malaval L, Monfoulet L, Fabre T, Pothuaud L, Bareille R, Miraux S, Thiaudiere E, Raffard G, Franconi J M, Lafage-Proust M H et al. 2009b. Absence of bone sialoprotein (bsp) impairs cortical defect repair in mouse long bone. Bone. 45(5):853-861.
Malaval L, Wade-Gueye N M, Boudiffa M, Fei J, Zirngibl R, Chen F, Laroche N, Roux J P, Burt-Pichat B, Duboeuf F et al. 2008. Bone sialoprotein plays a functional role in bone formation and osteoclastogenesis. J Exp Med. 205(5):1145-1153.
Miwa H E, Gerken T A, Jamison O, Tabak L A. 2010. Isoform-specific o-glycosylation of osteopontin and bone sialoprotein by polypeptide n-acetylgalactosaminyltransferase-1. J Biol Chem. 285(2):1208-1219.
Monfoulet L, Malaval L, Aubin J E, Rittling S R, Gadeau A P, Fricain J C, Chassande O. 2010. Bone sialoprotein, but not osteopontin, deficiency impairs the mineralization of regenerating bone during cortical defect healing. Bone. 46(2):447-452.
Nagasaki A, Nagasaki K, Kear B D, Tadesse W D, Thumbigere-Math V, Millan J L, Foster B L, Somerman M J. 2021. Delivery of alkaline phosphatase promotes periodontal regeneration in mice. J Dent Res. 100(9):993-1001.
Shimizu M, Sasaki T, Ishihara A, Furuya R, Kawawa T. 1998. Bone wound healing after maxillary molar extraction in ovariectomized aged rats. J Electron Microsc (Tokyo). 47(5):517-526.
Soenjaya Y, Foster B L, Nociti F H, Jr., Ao M, Holdsworth D W, Hunter G K, Somerman M J, Goldberg H A. 2015. Mechanical forces exacerbate periodontal defects in bsp-null mice. J Dent Res. 94(9):1276-1285.
Tanaka S, Shimizu M, Debari K, Furuya R, Kawawa T, Sasaki T. 2001. Acute effects of ovariectomy on wound healing of alveolar bone after maxillary molar extraction in aged rats. Anat Rec. 262(2):203-212.
Tye C E, Hunter G K, Goldberg H A. 2005. Identification of the type i collagen-binding domain of bone sialoprotein and characterization of the mechanism of interaction. J Biol Chem. 280(14):13487-13492.
Tye C E, Rattray K R, Warner K J, Gordon J A, Sodek J, Hunter G K, Goldberg H A. 2003. Delineation of the hydroxyapatite-nucleating domains of bone sialoprotein. J Biol Chem. 278(10):7949-7955.
Van der Velden U. 1984. Effect of age on the periodontium. J Clin Periodontol. 11(5):281-294.
Wade-Gueye N M, Boudiffa M, Vanden-Bossche A, Laroche N, Aubin J E, Vico L, Lafage-Proust M H, Malaval L. 2012. Absence of bone sialoprotein (bsp) impairs primary bone formation and resorption: The marrow ablation model under pth challenge. Bone. 50(5):1064-1073.
Wang J, Zhou H Y, Salih E, Xu L, Wunderlich L, Gu X, Hofstaetter J G, Torres M, Glimcher M J. 2006. Site-specific in vivo calcification and osteogenesis stimulated by bone sialoprotein. Calcif Tissue Int. 79(3):179-189.
Wazen R M, Tye C E, Goldberg H A, Hunter G K, Smith C E, Nanci A. 2007. In vivo functional analysis of polyglutamic acid domains in recombinant bone sialoprotein. J Histochem Cytochem. 55(1):35-42.
Xu L, Zhang Z, Sun X, Wang J, Xu W, Shi L, Lu J, Tang J, Liu J, Su X. 2017. Glycosylation status of bone sialoprotein and its role in mineralization. Exp Cell Res. 360(2):413-420.

Yuan X, Pei X, Zhao Y, Tulu U S, Liu B, Helms J A. 2018. A wnt-responsive pdl population effectuates extraction socket healing. J Dent Res. 97(7):803-809.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method for promoting alveolar bone healing in a subject in need thereof, the method comprising contacting the alveolar bone with a composition comprising a bone sialoprotein.

2. The method of claim 1, wherein promoting alveolar bone healing in a subject in need thereof comprises improving alveolar bone density at a locus in the subject, improving alveolar bone fraction at a locus in the subject, improving alveolar bone fraction at a locus in the subject, or a combination thereof.

3. The method of any of claims 1-2, wherein the subject has lost a tooth or had a tooth removed resulting in an empty socket in the alveolar bone; and
wherein contacting the alveolar bone with the composition comprising the bone sialoprotein comprises applying the composition comprising the bone sialoprotein to the empty socket.

4. The method of claim 3, wherein applying the composition comprising the bone sialoprotein to the empty socket comprises packing the empty socket with the composition comprising the bone sialoprotein.

5. The method of any of claims 3-4, wherein the method further comprises suturing gingival tissues to secure the composition comprising the bone sialoprotein within the empty socket.

6. The method of any of claims 3-5, wherein the composition comprising the bone sialoprotein stimulates the growth of alveolar bone to serve as a foundation for a dental implant at the site of the empty socket.

7. A method for performing a tooth extraction in a subject, the method comprising:
extracting a tooth from the subject, resulting in an empty socket in the subject's alveolar bone;
packing the empty socket with a composition comprising a bone sialoprotein; and
suturing gingival tissues to secure the composition comprising the bone sialoprotein within the empty socket.

8. A method for maintaining alveolar bone health in a subject in need thereof, the method comprising contacting the alveolar bone with a composition comprising a bone sialoprotein.

9. The method of claim 8, wherein the subject has lost a tooth or had a tooth removed resulting in an empty socket in the alveolar bone; and
wherein contacting the alveolar bone with the composition comprising the bone sialoprotein comprises applying the composition comprising the bone sialoprotein to the empty socket.

10. The method of claim 9, wherein applying the composition comprising the bone sialoprotein to the empty socket comprises packing the empty socket with the composition comprising the bone sialoprotein.

11. The method of any of claims 9-10, wherein the method further comprises suturing gingival tissues to secure the composition comprising the bone sialoprotein within the empty socket.

12. The method of any of claims 9-11, wherein the composition comprising the bone sialoprotein maintains sufficient alveolar bone at the empty socket to serve as a foundation for a dental implant at the site of the empty socket.

13. A method for treating periodontal disease in a subject in need thereof, the method comprising delivering a composition comprising a bone sialoprotein locally to a site affected by the periodontal disease.

14. The method of claim 13, wherein delivering the composition comprising the bone sialoprotein locally to the site affected by the periodontal disease comprises placement of the composition comprising the bone sialoprotein at the gum line of the site affected by the periodontal disease.

15. The method of any of claims 13-14, wherein the periodontal disease is in a hard tissue destruction phase.

16. The method of any of claims 13-15, wherein the composition comprising the bone sialoprotein is delivered in an effective amount to improve alveolar bone density at the site affected by the periodontal disease, improve alveolar bone fraction at the site affected by the periodontal disease, improve alveolar bone fraction at the site affected by the periodontal disease, or a combination thereof.

17. The method of any of claims 13-16, wherein the composition comprising the bone sialoprotein is delivered in an effective amount to regenerate cementum at the site affected by the periodontal disease.

18. A method for improving integration of a dental implant within the alveolar bone of a subject, the method comprising contacting the dental implant, the alveolar bone of the subject, or a combination thereof with a composition comprising a bone sialoprotein.

19. A method for treating a traumatic injury to alveolar bone in a subject in need thereof, the method comprising contacting the alveolar bone in an area proximate to the traumatic injury with a composition comprising a bone sialoprotein.

20. The method of any of claims 1-19, wherein the composition comprising the bone sialoprotein comprises the bone sialoprotein dissolved or dispersed in a pharmaceutically acceptable carrier.

21. The method of claim 21, wherein the pharmaceutically acceptable carrier comprises a polymeric matrix.

22. The method of claim 21, wherein the polymeric matrix comprises a biocompatible polymer.

23. The method of any of claims 21-22, wherein the polymeric matrix comprises a biodegradable polymer.

24. The method of any of claims 21-23, wherein the polymeric matrix comprises a polymer selected from the group consisting of polyesters, ionomers, poly(amino acids), poly(peptides), proteins, polyvinyl acetate, polyvinyl alcohols polyacrylates, polyorthoesters, polyanhydrides, fibrins, starches, polysaccharides such as alginate, or a combination thereof.

25. The method of any of claims 21-24, wherein the polymeric matrix comprises collagen.

26. The method of any of claims 1-25, wherein the composition is a gel at room temperature.

27. The method of any of claims 1-26, wherein the composition is a solid at room temperature.

28. The method of claim 27, wherein the composition is formed as a film, a fiber, a filament, a sheet, a thread, a cylindrical implant, a powder, a particulate, an asymmetrically-shaped implant, or a fibrous mesh.

29. The method of claim 28, wherein the method further comprises forming the composition into its selected form and shape.

30. The method of any of claims 1-29, wherein the bone sialoprotein comprises a recombinant bone sialoprotein.

31. The method of any of claims 1-30, wherein the bone sialoprotein lacks one or more post-translational modifications characteristic of native bone sialoprotein protein isolated from human.

32. The method of any of claims 1-31, wherein the composition further comprises a physiological mineral source for bone formation, such as a calcium phosphate.

33. The method of claim 32, wherein the composition further comprises tricalcium phosphate, hydroxyapatite, gypsum, or a combination thereof.

34. The method of any of claims 1-33, wherein the composition further comprises an osteogenic or other growth factor, such as osteogenin, bone morphogenetic protein, parathyroid hormone, or TGF-β.

35. The method of any of claims 1-34, wherein the composition further comprises an antimicrobial agent, such as metronidazole, tobramycin, gentamicin, or vancomycin.
